(19) United States
(12) Patent Application Publication
(10) Pub. No.: US 2025/0120961 A1
(43) Pub. Date: Apr. 17, 2025

(54) P53 MODULATORS

(71) Applicant: Nested Therapeutics, Inc., Cambridge, MA (US)

(72) Inventors: David Belanger, Cambridge, MA (US); Yongxin Han, Cambridge, MA (US); Jeffrey Hale, Cambridge, MA (US); Daniel F. Ortwine, Cambridge, MA (US); Aysegul Ozen, Cambridge, MA (US); Matthew Hurley, Cambridge, MA (US); Kamran Haider, Cambridge, MA (US); Mark E. Fitzgerald, Cambridge, MA (US); Michael Hale, Cambridge, MA (US)

(21) Appl. No.: 18/757,176

(22) Filed: Jun. 27, 2024

Related U.S. Application Data
(60) Provisional application No. 63/523,510, filed on Jun. 27, 2023.

(51) Int. Cl. A61K 31/454; A61K 31/4025; A61K 31/4155; A61K 31/4196; A61K 31/422; A61K 31/4245; A61K 31/428; A61K 31/4439; A61K 31/4545; C07D 401/12; C07D 401/14; C07D 409/12; C07D 409/14; C07D 413/14; C07D 471/04

(52) U.S. Cl. CPC A61K 31/454; A61K 31/4025; A61K 31/4155; A61K 31/4196; A61K 31/422; A61K 31/4245; A61K 31/428; A61K 31/4439; A61K 31/4545; C07D 401/12; C07D 401/14; C07D 409/12; C07D 409/14; C07D 413/14; C07D 471/04

(57) ABSTRACT

Disclosed are p53 modulators represented by the following structural formula I:

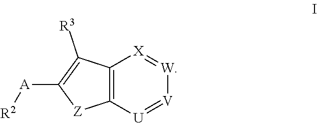

The variables in structural formula I are described herein. Also disclosed are method of treating cancer in a subject with the disclosed p53 modulators, particularly cancers with dysfunctional p53.

### RELATED APPLICATIONS

This application claims priority to U.S. Application No. 63/523,510, filed on Jun. 27, 2023, the entire contents of which are incorporated herein by reference.

### BACKGROUND

The p53 protein is a tetrameric transcription factor that prevents mutation to the genome by regulating the expression of a subgroup of target genes. Activation of p53 initiates pathways involved in apoptosis, DNA repair, cell cycle arrest, anti-angiogenesis, and senescence in order to avoid propagation of damaged cells.

Tumor suppressor protein p53 is also a transcription factor that plays an important role in human cancers. Tumor initiation and maintenance depend upon inactivation of p53 pathways, which otherwise would deter uncontrolled cell growth. Consequently, p53 is the most frequently mutated gene in human cancers. It was estimated that more than half of all human tumors have mutant p53. The majority of those tumors that harbor mutant p53 are found to express full-length p53 protein with a single residue missense mutation in the p53 DNA-binding core domain (DBD). However, structural mutations remote from the DBD are also common. The Y220C mutation, which occurs in approximately 1.5% all human cancers, is such a structurally destabilizing mutation at codon 220, resulting in structural instability and loss of DNA binding at body temperature due to loss of beneficial lipophilic contacts of the tyrosine-220 residue as seen in wt-p53. The p53 Y220C mutation is associated with many cancers, including breast cancer, non-small cell lung cancer, colorectal cancer, pancreatic cancer, and ovarian cancer.

The frequency and aggressive nature of cancers exhibiting p53 malfunction coupled with the potential benefits of restoring wild type p53 function has driven a widespread effort to identify compounds that restore normal p53 expression and activity. Nonetheless, there is still a critical need in the art for the development of new small molecule reactivators targeting p53 mutants (e.g., Y220C mutant) with high specificity and activity as well as low toxicity.

### SUMMARY

Provided herein are compounds, or pharmaceutically acceptable salts thereof, and compositions which bind to the Y220C pocket, stabilize the mutant protein and restore ability of the mutant protein to bind to DNA at physiologically relevant temperatures. For example, Biological Example 1 provides data showing that the disclosed compounds can bind to the Y220C pocket, stabilize the mutant protein and restore ability of the mutant protein to bind to DNA at physiologically relevant temperatures. Also disclosed are methods of using the compounds and compositions described herein for treating cancer.

A first embodiment of the disclosure is a compound represented by the following structural formula:

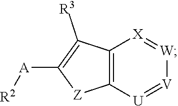

or a pharmaceutically acceptable salt thereof, wherein:
   A is represented by

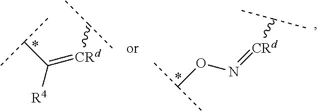

wherein “*” indicates the point of attachment to R²;
Z is S, O, or NR^{3a};
U, V, W and X are independently CR⁵ or N, provided that one of U, V, W and X is C—Y—R¹;
Y is O, NH, N(C₁₋₄alkyl), NHCH₂\hat{ }, OCH₂\hat{ }, S or CH₂, wherein “\hat{ }” indicates the point of attachment to R¹;
R¹ is (CH₂)ₙOR¹¹, (CH₂)ₙN(R¹¹)₂, (CH₂)ₙCN, (CH₂)ₙC(O)R¹¹, (CH₂)ₙC(O)OR¹¹, (CH₂)ₙC(S)R¹¹, (CH₂)ₙC(S)OR¹¹, (CH₂)ₙC(O)N(R¹¹)₂, (CH₂)ₙNHC(O)R¹¹, (CH₂)ₙNHC(O)OR¹¹, (CH₂)ₙOC(O)N(R¹¹)₂, (CH₂)ₙC(S)N(R¹¹)₂, (CH₂)ₙNHC(S)R¹¹, (CH₂)ₙNHC(S)OR¹¹, (CH₂)ₙOC(S)N(R¹¹)₂, (CH₂)ₙNHS(O)ᵢR¹¹, (CH₂)ₙS(O)ᵢN(R¹¹)₂, C₁₋₆ alkyl, C₃₋₈ cycloalkyl, C₂₋₆ alkenyl, C₂₋₆ alkynyl, C₆₋₁₀ aryl, 5-10 membered heteroaryl, or 4-10 membered heterocyclyl, wherein said alkyl, alkenyl, alkynyl, cycloalkyl, aryl, heteroaryl and heterocyclyl are optionally and independently substituted by one or more R²¹;
R² is independently (CH₂)ₘOR¹², (CH₂)ₘN(R¹²)₂, (CH₂)ₘCN, (CH₂)ₘC(O)R¹², (CH₂)ₘC(O)OR¹², (CH₂)ₘC(S)R¹², (CH₂)ₘC(S)OR¹², (CH₂)ₘC(O)N(R¹²)₂, (CH₂)ₘNHC(O)R¹², (CH₂) NHC(O)OR¹², (CH₂)ₘOC(O)N(R¹²)₂, (CH₂)ₘC(S)N(R¹²)₂, (CH₂)ₘNHC(S)R¹², (CH₂)ₘNHC(S)OR¹², (CH₂)ₘOC(S)N(R¹²)₂, (CH₂)ₘNHS(O)ᵢR¹², (CH₂)ₘS(O)ᵢN(R¹²)₂, C₁₋₆ alkyl, (CH₂)ₘC₃₋₈ cycloalkyl, C₂₋₆ alkenyl, C₂₋₆ alkynyl, (CH₂)ₘC₆₋₁₀ aryl, 5-10 membered heteroaryl or (CH₂)ₘ(4-10 membered heterocyclyl), wherein:
   said alkyl, alkenyl and alkynyl are each optionally and independently substituted by one or more R^{22b};
   said aryl, and heteroaryl are each optionally and independently substituted by one or more R²²; and
   said cycloalkyl and heterocyclyl are each optionally substituted by one or more R^{22a};
when Z is O or S, R³ is H, halo, (CH₂)ₒOR¹³, (CH₂)ₒSR¹³, (CH₂)ₒN(R¹³)₂, (CH₂)ₒCN, (CH₂)ₒC(O)R¹³, CH₂)ₒC(O)OR¹³, (CH₂)ₒC(S)R¹³, (CH₂)ₒC(S)OR¹³, (CH₂)ₒC(S)OR¹³ (CH₂)ₒC(O)N(R¹³)₂, (CH₂)ₒNHC(O)R¹³, (CH₂)ₒNHC(O)OR¹³, (CH₂)ₒOC(O)N(R¹³)₂, (CH₂)ₒC(S)N(R¹³)₂, (CH₂)ₒNHC(S)R¹³, (CH₂)ₒNHC(S)OR¹³, (CH₂)ₒOC(S)N(R¹³)₂, (CH₂)ₒNHS(O)ᵢR¹³, (CH₂)ₒS(O)ᵢN(R¹³)₂, C₁₋₆ alkyl, C₃₋₈ cycloalkyl, C₂₋₆ alkenyl or C₂₋₆ alkynyl, wherein said alkyl, alkenyl, alkynyl and cycloalkyl are each optionally and independently substituted by one or more R²³;
when Z is NR^{3a}, R³ is H, halo, (CH₂)ₒOR¹³, (CH₂)ₒSR¹³, C₁₋₆ alkyl or C₃₋₆ cycloalkyl, wherein said alkyl and cycloalkyl are each optionally and independently substituted by one or more R²³;
R^{3a} is H, (CH₂)ₒOR¹³, (CH₂)ₒSR¹³, (CH₂)ₒN(R¹³)₂, (CH₂)ₒCN, (CH₂)ₒC(O)R¹³, CH₂)ₒC(O)OR¹³, (CH₂)ₒC(S)R¹³, (CH₂)ₒC(S)OR¹³, (CH₂)ₒC(S)OR¹³ (CH₂)ₒC(O)N(R¹³)₂, (CH₂)ₒNHC(O)R¹³, (CH₂)ₒNHC(O)OR¹³, (CH₂)ₒOC(O)N(R¹³)₂, (CH₂)ₒC(S)N(R¹³)₂, (CH₂)ₒNHC(S)R¹³, (CH₂)ₒNHC(S)OR¹³, (CH₂)ₒOC(S)N(R¹³)₂, (CH₂)ₒNHS(O)ᵢR¹³, (CH₂)ₒS(O)ᵢN(R¹³)₂, C₁₋₆ alkyl, C₃₋₈ cycloalkyl, C₂₋₆ alkenyl or C₂₋₆ alkynyl, wherein said alkyl, alkenyl, alkynyl and cycloalkyl are each optionally and independently substituted by one or more R²³;
R⁴ is H, halo, CN, C₁₋₆ alkyl, or C₃₋₈ cycloalkyl, wherein said alkyl, and cycloalkyl are each optionally and independently substituted by one or more R²⁴; or
R² and R⁴ are optionally taken together with their intervening atom to form a C₃₋₁₀ non-aromatic carbocyclyl, or 4-10 membered heterocyclyl, wherein said carbocyclyl or heterocyclyl are each optionally and independently substituted by one or more R²²; and
each R⁵ is independently H, halo, (CH₂)ₚOR¹⁵, (CH₂)ₚN(R¹⁵)₂, (CH₂)ₚCN, (CH₂)ₒC(O)R¹⁵, (CH₂)ₚC(O)OR¹⁵, (CH₂)ₚC(S)R¹⁵, (CH₂)ₚC(S)OR¹⁵, (CH₂) PC(O)N(R¹⁵)₂, (CH₂)ₚNHC(O)R¹⁵, (CH₂)ₚNHC(O)OR¹⁵, (CH₂)ₚOC(O)N(R¹⁵)₂, (CH₂)ₚC(S)N(R¹⁵)₂, (CH₂)ₚNHC(S)R¹⁵, (CH₂)ₚNHC(S)OR¹⁵, (CH₂)ₚOC(S)N(R¹⁵)₂, (CH₂)ₚNHS(O)ᵢR¹⁵, (CH₂)ₚS(O)ᵢN(R¹⁵)₂, C₁₋₆ alkyl, C₃₋₈ cycloalkyl, C₂₋₆ alkenyl, C₂₋₆ alkynyl, C₆₋₁₀ aryl, 5-10 membered heteroaryl or 4-10 membered heterocyclyl, wherein said alkyl, alkenyl, alkoxy, alkynyl, cycloalkyl, aryl, heteroaryl or heterocyclyl is optionally and independently substituted by one or more R²⁵;
R¹¹, R¹², R¹³, and R¹⁵ are each independently H, C₁₋₆ alkyl, C₃₋₈ cycloalkyl, (CH₂) C₃₋₈ cycloalkyl, C₆₋₁₀ aryl, 5-10 membered heteroaryl or 4-10 membered heterocyclyl, wherein said alkyl, cycloalkyl, aryl, heteroaryl and heterocyclyl are optionally and independently substituted by one or more R³⁰; or N(R¹¹)₂ and N(R¹²)₂ are independently a 4-10 membered heterocyclyl, wherein the heterocyclyl represented by N(R¹¹) is optionally substituted with one or more R²¹ and the heterocyclyl represented by N(R¹²) is optionally substituted with one or more R²²;
R²¹, R²², R²³, R²⁴ and R²⁵ are each independently halo, CN, NO₂, (CH₂)ₙOR^{a}, (CH₂)ₙNR^{b}R^{c}, S(O)ᵢR^{b}, (═NH)NR^{b}R^{c}, NHS(O)ᵢR^{c}, S(O)ᵢNR^{b}R^{c}, C(═O)OR^{b}, OC(═O)OR^{b}, C(═S)OR^{b}, O(C═S)R^{b}, C(═O)NR^{b}R^{c}, NR^{b}C(═O)R^{c}, C(═S)NR^{b}R^{c}, NHC(═S)R^{c}, NH(C═O)OR^{c}, O(C═O)NR^{b}R^{c}, NH(C═S)OR^{c}, O(C═S)NR^{b}R^{c}, NH(C═O)NR^{b}R^{c}, NH(C═S)NR^{b}R^{c}, C(═S)R^{b}, C(═O)R^{b}, halo(C₁-C₅)alkyl, (C₁-C₅)alkyl, (C₂-C₅)alkenyl or (C₂-C₅)alkynyl;
each R^{22a} is independently halo, CN, NO₂, (CH₂)ₙOR^{a}, (CH₂)ₙNR^{b}R^{c}, S(O)ᵢR^{b}, (═NH)NR^{b}R^{c}, NHS(O)ᵢR^{c}, S(O)ᵢNR^{b}R^{c}, C(═O)OR^{b}, C(═S)OR^{b}, O(C═S)R^{b}, C(═O)NR^{b}R^{c}, NHC(═O)R^{c}, C(═S)NR^{b}R^{c}, NHC(═S)R^{c}, NH(C═O)OR^{c}, O(C═O)NR^{b}R^{c}, NH(C═S)OR^{c}, O(C═S)NR^{b}R^{c}, NH(C═O)NR^{b}R^{c}, NH(C═S)NR^{b}R^{c}, C(═S)R^{b}, C(═O)R^{b}, halo(C₁-C₅)alkyl, (C₁-C₅)alkyl, (C₂-C₅)alkenyl or (C₂-C₅)alkynyl, or two R^{22a} groups on the same ring atom taken together with their intervening atom are a (C₃₋₆)spirocycloalkyl or oxo, or two R^{22a} on adjacent ring atoms, taken together their intervening atoms form a phenyl, or a 5-6 membered heteroaryl, wherein said phenyl or heteroaryl is optionally substituted with one or more groups selected from halo, methyl, halomethyl, methoxy or halomethoxy;
each R^{22b} is independently halo, CN, NO₂, (CH₂)ₙOR^{a}, (CH₂)ₙNR^{b}R^{c}, S(O)ᵢR^{b}, (═NH)NR^{b}R^{c}, NHS(O)ᵢR^{c}, S(O)ᵢNR^{b}R^{c}, C(═O)OR^{b}, OC(═O)OR^{b}, C(═S)OR^{b}, O(C═S)R^{b}, C(═O)NR^{b}R^{c}, NHC(═O)R^{c}, C(═S)NR^{b}R^{c}, NHC(═S)R^{c}, NH(C═O)OR^{c}, O(C═O)NR^{b}R^{c}, NH(C═S)OR^{c}, O(C═S)NR^{b}R^{c}, NH(C═O)NR^{b}R^{c}, NH(C═S)NR^{b}R^{c}, C(═S)R^{b}, C(═O)R^{b}, halo(C₁-C₅)alkyl, (C₁-C₅)alkyl, (C₂-C₅)alkenyl, (C₂-C₅)alkynyl, phenyl or 5-10 membered heteroaryl, wherein each of said phenyl and heteroaryl are optionally substituted with one or more (C₁-C₄)alkyl, (CH₂)ₙC₃₋₆ cycloalkyl, (CH₂)ₙOR^{a}, (C₁-C₄)haloalkyl or halo;
each R³⁰ is independently halo, C₁₋₄ alkyl, C₁₋₄ fluoroalkyl, C₃₋₅ Cycloalkyl, (CH₂)₀₋₁OR^{a}, S(O)ᵢR^{b}, or (CH₂)₀₋₁NR^{a}R^{b};
each R^{a}, R^{b} and R^{c} is independently —H, C₁₋₄ alkyl, or benzyl; wherein benzyl is optionally substituted with one or more methyl or methoxyl;
R^{d} is H or C₁₋₄ alkyl; and
each n, m, o, p and i are independently 0, 1 or 2.

Another embodiment of the disclosure is a pharmaceutical composition comprising a pharmaceutically acceptable carrier, excipient, or diluent, and a compound disclosed herein or a pharmaceutically acceptable salt thereof.

Another embodiment of the disclosure is a method of re-activating p53 Y220C mutant in a subject in need thereof, comprising contacting p53 Y220C mutant with an effective amount of the compound disclosed herein or a pharmaceutically acceptable salt thereof or a pharmaceutical composition comprising a compound disclosed herein or a pharmaceutically acceptable salt thereof.

Another embodiment of the disclosure is the use of a compound disclosed herein or a pharmaceutically acceptable salt thereof or a pharmaceutical composition comprising a compound disclosed herein or a pharmaceutically acceptable salt thereof for the manufacture of a medicament for re-activating p53 Y220C mutant in a subject in need thereof.

Another embodiment of the disclosure is a compound disclosed herein or a pharmaceutically acceptable salt thereof or a pharmaceutical composition comprising a compound disclosed herein or a pharmaceutically acceptable salt thereof for re-activating p53 Y220C mutant in a subject in need thereof.

Another embodiment of the disclosure is a method of treating a p53 Y220C mutant-dependent disorder or disease (e.g., treating a cancer) in a subject in need thereof, comprising administering to the subject an effective amount of a compound disclosed herein or a pharmaceutically acceptable salt thereof or a pharmaceutical composition comprising the compound(s).

Another embodiment of the disclosure is the use of a compound disclosed herein or a pharmaceutically acceptable salt thereof or a pharmaceutical composition comprising the compound(s), for the preparation of a medicament for treating a p53 Y220C mutant-dependent disorder or disease (e.g., treating a cancer) in a subject in need thereof.

Another embodiment of the disclosure is a compound disclosed herein or a pharmaceutically acceptable salt thereof or a pharmaceutical composition comprising the compound(s), for use in treating a p53 Y220C mutant-dependent disorder or disease (e.g., treating a cancer) in a subject in need thereof.

### DETAILED DESCRIPTION

The present invention provides compounds, compositions and methods for restoring wild-type function of mutant p53. The compounds of the present invention can bind to mutant p53 and restore the ability of the p53 mutant to bind DNA. The restoration of activity of the p53 mutant can allow for the activation of downstream effectors of p53 leading to inhibition of cancer progression. The present invention further provides a method for treating a disease or condition related to p53 mutant protein.

Example embodiments include:
First embodiment: a compound represented by Formula I:

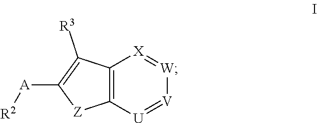

or a pharmaceutically acceptable salt thereof. The variables in Formula I are described in the summary above.
Second embodiment: a compound represented by Formula II:

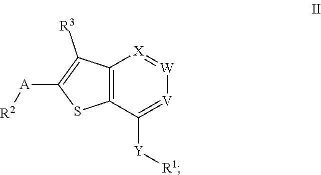

or a pharmaceutically acceptable salt thereof, wherein the definitions for the variables in Formula II are as defined in the first embodiment.
Third embodiment: a compound represented by Formula III:

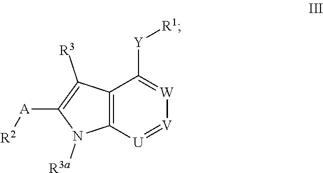

or a pharmaceutically acceptable salt thereof, wherein the definitions for the variables in Formula III are as defined in the first embodiment.
Fourth embodiment: a compound represented by Formula IV:

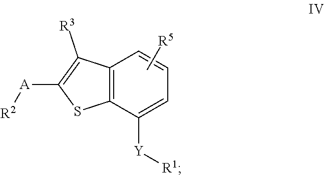

or a pharmaceutically acceptable salt thereof, wherein the definitions for the variables in Formula IV are as defined in the first embodiment.
Fifth embodiment: a compound represented by Formulae V, VI, VII, VIII, IX, or X:

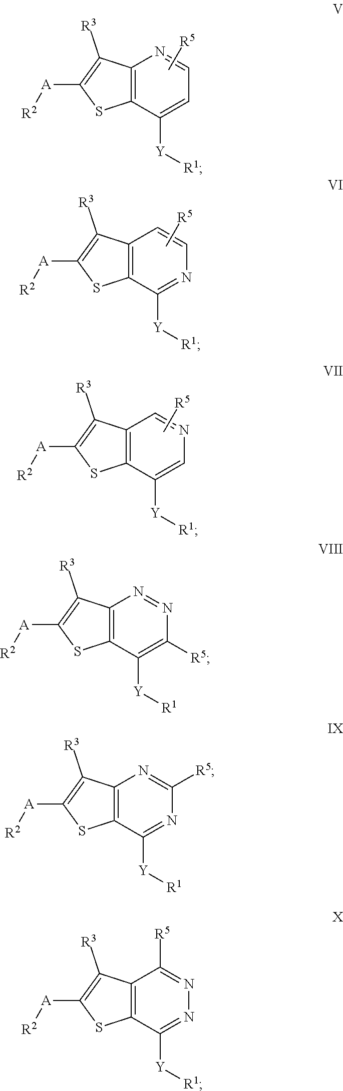

or a pharmaceutically acceptable salt thereof, wherein the definitions for the variables in Formulae V, VI, VII, VIII, IX, and X are as defined in the first embodiment.
Sixth embodiment: a compound represented by Formula XI:

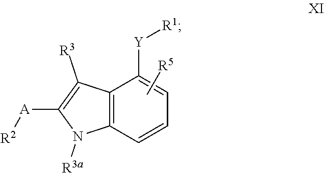

or a pharmaceutically acceptable salt thereof, wherein the definitions for the variables in Formula XI are as defined in the first embodiment.
Seventh embodiment: a compound represented by Formulae XII, XIII, XIV, XV, XVI or XVII:

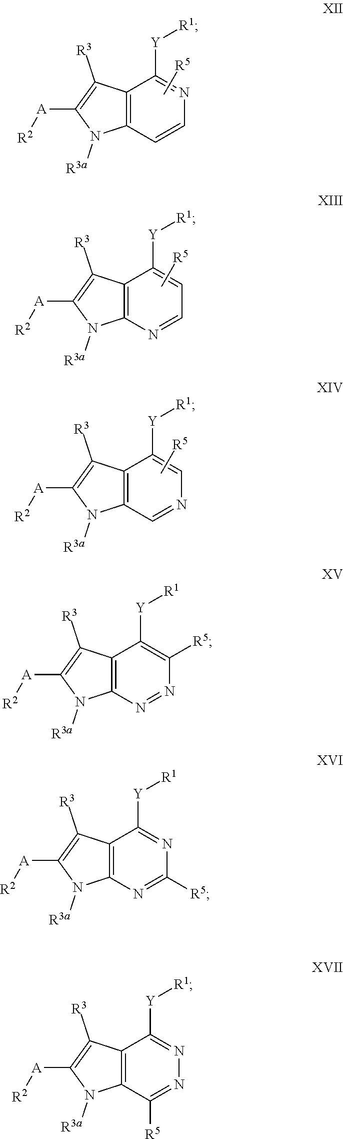

or a pharmaceutically acceptable salt thereof, wherein the definitions for the variables in Formulae XII, XIII, XIV, XV, XVI or XVII are as defined in the first embodiment.
Eighth embodiment: a compound represented by Formulae I, II, III, IV, or XI, or a pharmaceutically acceptable salt thereof, wherein A is represented by

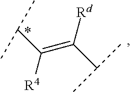

wherein “*” indicates the point of attachment to R², wherein the definitions for the other variables in Formulae I, II, III, IV, and XI, are as defined in the first embodiment.
Ninth embodiment: a compound represented by Formulae I, II, III, IV, or XI, or a pharmaceutically acceptable salt thereof, wherein A is represented by

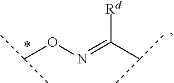

wherein “*” indicates the point of attachment to R², wherein the definitions for the other variables in Formulae I, II, III, IV, and XI, are as defined in the first embodiment.
Tenth embodiment: a compound represented by Formulae I, II, III, IV, or XI, or a pharmaceutically acceptable salt thereof, wherein Y is O, NH or NHCH₂\hat{ }, wherein the definitions for the other variables in Formulae I, II, III, IV, and XI, are as defined in the first, eighth, and ninth embodiments.
Eleventh embodiment: a compound represented by Formulae I, II, III, IV, or XI, or a pharmaceutically acceptable salt thereof, wherein Y is NH, wherein the definitions for the other variables in Formulae I, II, III, IV, and XI, are as defined in the first, eighth, and ninth embodiments.
Twelfth embodiment: a compound represented by Formulae I, II, or IV, or a pharmaceutically acceptable salt thereof, wherein R³ is (CH₂)ₒC(O)(C₁₋₆ alkyl), (CH₂)ₒS(C₁₋₆ alkyl), C₁₋₆ alkyl, (CH₂)ₒC₃₋₈ cycloalkyl, C₂₋₆ alkenyl, or C₂₋₆ alkynyl, wherein o is 0 or 1; and the C₁₋₆ alkyl represented by R³ or the C₁₋₆ alkyl, C₃₋₈ cycloalkyl, C₂₋₆ alkenyl and C₂₋₆ alkynyl in the group represented by R³ are optionally substituted with hydroxyl and/or one or more halo groups, and wherein the definitions for the other variables in Formulae I, II, or IV are as defined in the first, eighth, ninth, tenth, and eleventh embodiments.
Thirteenth embodiment: a compound represented by Formulae I, II, or IV, or a pharmaceutically acceptable salt thereof, wherein R³ is C₁₋₄ alkyl or S(C₁₋₄ alkyl), wherein the C₁₋₄ alkyl represented by R³ or the C₁₋₄ alkyl in the group represented by R³ is substituted by one or more fluoro, wherein the definitions for the other variables in Formulae I, II, or IV are as defined in the first, eighth, ninth, tenth, and eleventh embodiments.
Fourteenth embodiment: a compound represented by Formulae I, II, or IV, or a pharmaceutically acceptable salt thereof, wherein R³ is CH₂CF₃, wherein the definitions for the other variables in Formulae I, II, or IV are as defined in the first, eighth, ninth, tenth, and eleventh embodiments.
Fifteenth embodiment: a compound represented by Formulae I, II, or IV, or a pharmaceutically acceptable salt thereof, wherein R³ is SCF₃, wherein the definitions for the other variables in Formulae I, II, or IV are as defined in the first, eighth, ninth, tenth, and eleventh embodiments.
Sixteenth embodiment: a compound represented by Formulae I, III, or XI, or a pharmaceutically acceptable salt thereof, wherein
R³ is H; and
R^{3a} is (CH₂)ₒC(O)(C₁₋₆ alkyl), (CH₂)ₒS(C₁₋₆ alkyl), C₁₋₆ alkyl, (CH₂)ₒC₃₋₈ cycloalkyl, C₂₋₆ alkenyl, or C₂₋₆ alkynyl, wherein o is 0 or 1; and the C₁₋₆ alkyl represented by R^{3a} or the C₁₋₆ alkyl, C₃₋₈ cycloalkyl, C₂₋₆ alkenyl and C₂₋₆ alkynyl in the group represented by R^{3a} are optionally substituted with hydroxyl and/or one or more halo groups;
the remainder of variables in Formulae I, III, or XI are as defined in the first, eighth, ninth, tenth, and eleventh embodiments.
Seventeenth embodiment: a compound represented by Formulae I, III, or XI, or a pharmaceutically acceptable salt thereof, wherein R^{3a} is C₁₋₄ alkyl or S(C₁₋₄ alkyl), wherein the C₁₋₄ alkyl represented by R^{3a} or the C₁₋₄ alkyl in the group represented by R^{3a} is substituted by one or more fluoro, wherein the definitions for the other variables in Formulae I, III, or XI are as defined in the first, eighth, ninth, tenth, eleventh, and sixteenth embodiments.
Eighteenth embodiment: a compound represented by Formulae I, III, or XI, or a pharmaceutically acceptable salt thereof, wherein R^{3a} is CH₂CF₃. Alternatively, R^{3a} is SCF₃. The definitions for the other variables in these two alternatives in Formulae I, III, or XI are as defined in the first, eighth, ninth, tenth, eleventh, and sixteenth embodiments.
Nineteenth embodiment: a compound represented by any of the Formulae I to XVII, or a pharmaceutically acceptable salt thereof, wherein each R⁵ is independently H, C₁₋₄ alkyl, or halo, wherein the definitions for the other variables in Formulae I to XVII are as defined in the first, eighth, ninth, tenth, eleventh, twelfth, thirteenth, fourteenth, fifteenth, sixteenth, seventeenth, and eighteenth embodiments.
Twentieth embodiment: a compound represented by any of the Formulae I to XVII, or a pharmaceutically acceptable salt thereof, wherein R⁵ is H, wherein the definitions for the other variables in Formulae I to XVII are as defined in the first, eighth, ninth, tenth, eleventh, twelfth, thirteenth, fourteenth, fifteenth, sixteenth, seventeenth, and eighteenth embodiments.
Twenty-first embodiment: a compound represented by Formulae I, II, III, IV, or XI, or a pharmaceutically acceptable salt thereof, wherein
R² is (CH₂)ₘOR¹², (CH₂)ₘN(R¹²)₂, (CH₂)ₘCN, (CH₂)ₘC(O)N(R¹²)₂, (CH₂)ₘNHC(O)R¹², (CH₂)ₘNHC(O)OR¹², (CH₂)ₘNHS(O)ᵢR¹², C₁₋₆ alkyl, (CH₂)ₘC₃₋₈ cycloalkyl, (CH₂)ₘC₆₋₁₀ aryl, 5-10 membered heteroaryl or (CH₂)ₘ(4-10 membered heterocyclyl), wherein
said alkyl is optionally substituted by one or more R^{22b};
said aryl and heteroaryl are each optionally and independently substituted by one or more R²²; and
said cycloalkyl and heterocyclyl are each optionally substituted by one or more R^{22a}; and
R² and R⁴ are optionally taken together with their intervening atom to form a C₃₋₆ cycloalkyl, or 4-6 membered heterocyclyl containing one ring nitrogen atom or one ring oxygen atom, wherein said cycloalkyl or heterocyclyl are each optionally and independently substituted by one or more R²²;
R⁴ is H, halo, or C₁₋₄ alkyl;
R¹² are each independently H, C₁₋₆ alkyl, C₃₋₈ cycloalkyl or phenyl, 5-6 membered heteroaryl; wherein said alkyl, cycloalkyl, phenyl and heteroaryl are optionally and independently substituted by one or more R³⁰;
R²² are each independently halo, CN, NO₂, (CH₂)ₙOR^{a}, S(O)ᵢR^{b}, (CH₂)ₙNR^{b}R^{c}, NHS(O)ᵢR^{c}, C(═O)OR^{b}, C(═O)NR^{b}R^{c}, NHC(═O)R^{c}, NH(C═O)OR^{c}, O(C═O)NR^{b}R^{c}, C(═O)R^{b}, halo(C₁-C₅)alkyl, or (C₁-C₅)alkyl;
R^{22a} are each independently halo, CN, NO₂, (CH₂)ₙOR^{a}, (CH₂)ₙNR^{b}R^{c}, S(O)ᵢR^{b}, NHS(O)ᵢR^{c}, S(O)ᵢNR^{b}R^{c}, C(═O)OR^{b}, C(═O)NR^{b}R^{c}, NHC(═O)R^{c}, NH(C=O)OR^{c}, O(C═O)NR^{b}R^{c}, C(═O)R^{b}, halo(C₁-C₅)alkyl, (C₁-C₅)alkyl, or two R^{22a} groups on the same ring atom taken together with their intervening atom are a (C₃₋₆)spirocycloalkyl or oxo, or two R^{22a} on adjacent ring atoms, taken together their intervening atoms form a phenyl, or a 5-6 membered heteroaryl, wherein said phenyl or heteroaryl is optionally substituted with one or more groups selected from halo, methyl, halomethyl, methoxy or halomethoxy; and
R^{22b} are each independently halo, CN, NO₂, (CH₂)ₙOR^{a}, (CH₂)ₙNR^{b}R^{c}, NHS(O)ᵢR^{c}, OC(═O)OR^{b}, NHC(═O)R^{c}, NH(C═O)OR^{c}, O(C═O)NR^{b}R^{c}, NH(C═O)NR^{b}R^{c}, halo(C₁-C₅)alkyl, (C₁-C₅)alkyl, phenyl or 5-10 membered heteroaryl, where each said phenyl and heteroaryl are optionally substituted with one or more (C₁-C₄)alkyl, (CH₂)ₙC₃₋₆ cycloalkyl, (CH₂)ₙOR^{a}, (C₁-C₄)haloalkyl or halo;
the remainder of variables in Formulae I, II, III, IV, and XI, are as defined in the first, eighth, ninth, tenth, eleventh, twelfth, thirteenth, fourteenth, fifteenth, sixteenth, seventeenth, eighteenth, nineteenth, and twentieth embodiments.
Twenty-second embodiment: a compound represented by Formulae I, II, III, IV, or XI, or a pharmaceutically acceptable salt thereof, wherein
R² is (CH₂)ₘNHR¹², (CH₂)ₘC(O)N(R¹²)₂, (CH₂)ₘNHC(O)R¹², (CH₂)ₘNHC(O)OR¹², (CH₂)ₘNHS(O)ᵢR¹², C₁₋₄ alkyl, (CH₂)ₘC₃₋₆ cycloalkyl, (CH₂)ₘphenyl, 5-10 membered heteroaryl, or (CH₂)ₘ (4-6 membered heterocyclyl), wherein said heteroaryl is selected from pyrazolyl, imidazolyl, pyridyl, pyrimidyl, oxazolyl, oxadiazolyl, isoxazolyl, thiazolyl, triazolyl, tetrazolyl, pyridin-2-onyl, pyrazolopyridyl, benzimidazolyl, benzothiazolyl, indolyl, azaindolyl, indazolyl, azaindazolyl, or benzoxazolyl, and wherein said heterocyclyl is selected from pyrrolidinonyl, pyrrolidinyl, oxazolidin-2-onyl, 1,3-dioxol-2-onyl, pyridin-2 (1H)-onyl, tetrahydrofuranyl, tetrahydropyranyl, piperidinyl, piperazinyl, morpholinyl, azetidinyl, or oxetanyl, and wherein said alkyl is optionally substituted by one or more R^{22b};
said phenyl and heteroaryl are each optionally and independently substituted by one or more R²²;
said cycloalkyl and heterocyclyl are each optionally substituted by one or more R^{22a};
R¹² are each independently H, C₁₋₄ alkyl, C₃₋₆ cycloalkyl, phenyl, 5-6 membered heteroaryl, wherein said heteroaryl is selected from pyrazolyl, pyridyl, oxazolyl, isoxazolyl, thiazolyl, triazolyl, tetrazolyl, or imidazolyl, and wherein said alkyl, cycloalkyl, phenyl, or heteroaryl is optionally substituted with one or more groups selected from halo, C₁₋₄ alkyl, C₁₋₄ alkoxyl, S(O)ᵢR^{b}, C₁₋₄ fluoroalkyl, or C₃₋₅ cycloalkyl; and
R² and R⁴ are optionally taken together with their intervening atom to form a C₃₋₆cycloalkyl, or 4-6 membered heterocyclyl containing one ring nitrogen atom or one ring oxygen atom, wherein said cycloalkyl or heterocyclyl are each optionally and independently substituted by one or more R²²;
R⁴ is H, halo, or C₁₋₄ alkyl;
R²² are each independently halo, (CH₂)ₙOR^{a}, S(O)ᵢR^{b}, NR^{b}R^{c}, NHS(O)ᵢR^{c}, C(═O)OR^{b}, C(═O)NR^{b}R^{c}, NHC(═O)R^{c}, NH(C═O)OR^{c}, O(C═O)NR^{b}R^{c}, C(═O)R^{b}, halo(C₁-C₄)alkyl, or (C₁-C₄)alkyl;
R^{22a} are each independently halo, (CH₂)ₙOR^{a}, (CH₂)ₙNR^{b}R^{c}, S(O)ᵢR^{b}, NHS(O)ᵢR^{c}, S(O)ᵢNR^{b}R^{c}, C(═O)OR^{b}, C(═O)NR^{b}R^{c}, NHC(═O)R^{c}, NH(C═O)ORE, O(C═O)NR^{b}R^{c}, C(═O)R^{b}, halo(C₁-C₄)alkyl, or (C₁-C₄)alkyl; and
R^{22b} are each independently halo, (CH₂)ₙOR^{a}, (CH₂)ₙNR^{b}R^{c}, NHS(O)ᵢR^{c}, OC(═O)OR^{b}, NHC(═O)R^{c}, NH(C═O)OR^{c}, O(C═O)NR^{b}R^{c}, NH(C═O)NR^{b}R^{c}, halo(C₁-C₄)alkyl, (C₁-C₄)alkyl, pyrazolyl, pyridyl, pyridin-2-onyl, pyrazolopyridyl, oxadiazolyl, isoxazolyl, triazolyl, benzimidazolyl, benzothiazolyl, or phenyl, wherein said pyrazolyl, pyridyl, pyridin-2-onyl, pyrazolopyridyl, oxadiazolyl, isoxazolyl, triazolyl, benzimidazolyl, benzothiazolyl, or phenyl are each optionally and independently substituted with one or more (C₁-C₄)alkyl, CH₂ (C₃-C₆) cycloalkyl, (CH₂)ₙOR^{a}, (C₁-C₄)haloalkyl or halo;
the remainder of variables in Formulae I, II, III, IV, and XI, are as defined in the first, eighth, ninth, tenth, eleventh, twelfth, thirteenth, fourteenth, fifteenth, sixteenth, seventeenth, eighteenth, nineteenth, and twentieth embodiments.
Twenty-third embodiment: a compound represented by Formulae I, II, III, IV, or XI, or a pharmaceutically acceptable salt thereof, wherein
R² is:
   (CH₂)ₘNHR¹², (CH₂)ₘC(O)N(R¹²)₂, (CH₂)ₘNHC(O)R¹², (CH₂)ₘNHC(O)OR¹² or (CH₂)ₘNHS(O)ᵢR¹², wherein R¹² is H, C₁₋₄ alkyl, C₃₋₆cycloalkyl, pyrazolyl or phenyl, each optionally substituted with C₁₋₄ alkyl;
   (CH₂)ₘphenyl, wherein phenyl is optionally substituted with one or more groups selected from NR^{b}R^{c} and C(═O)NR^{b}R^{c};
   C₁₋₄ alkyl optionally substituted with one or more groups selected from halo, OR^{a}, NR^{b}R^{c}, pyrazolyl, pyridyl, pyridin-2-onyl, pyrazolopyridyl, oxadiazolyl, isoxazolyl, triazolyl, benzimidazolyl, benzothiazolyl, or phenyl, wherein said pyrazolyl, pyridyl, pyridin-2-onyl, pyrazolopyridyl, oxadiazolyl, isoxazolyl, triazolyl, benzimidazolyl, benzothiazolyl, or phenyl are each optionally and independently substituted with halo, methyl, cyclopropylmethyl, or methoxyl;
   (CH₂)_{0 or 1} C₃₋₆ cycloalkyl, (CH₂)_{0 or 1} 1,3-dioxol-2-onyl, (CH₂)_{0 or 1} pyrrolidinonyl, (CH₂)_{0 or 1} pyrrolidinyl, (CH₂)_{0 or 1} oxazolidin-2-onyl, (CH₂)_{0 or 1} tetrahydropyranyl, (CH₂)_{0 or 1} tetrahydrofuranyl, (CH₂)_{0 or 1} pyridin-2 (1H)-onyl or (CH₂)_{0 or 1} piperidinyl, each optionally and independently substituted by one or more groups selected from (CH₂)ₙOR^{a}, C₁₋₄ alkyl, C(O)OCH₂ (phenyl) and S(O)ᵢR^{b}; and
   R² and R⁴ are optionally taken together with their intervening atom to form a (C₃-C₆) cycloalkyl, piperidinyl or tetrahyropyranyl, each optionally and independently substituted by one or more methyl, C(O)(C₁₋₄ alkyl) or NHS(O)₂(C₁₋₄ alkyl); and
   R⁴ is H or C₁₋₄ alkyl;
   the remainder of variables in Formulae I, II, III, IV, and XI, are as defined in the first, eighth, ninth, tenth, eleventh, twelfth, thirteenth, fourteenth, fifteenth, sixteenth, seventeenth, eighteenth, nineteenth, and twentieth embodiments.
Twenty-fourth embodiment: a compound represented by Formulae I, II, III, IV, or XI, or a pharmaceutically acceptable salt thereof, wherein
R² is Me, (CH₂)ₘNHR¹², (CH₂)ₘC(O)N(R¹²)₂, (CH₂)ₘNHC(O)R¹², (CH₂)ₘNHC(O)OR¹², (CH₂)ₘNHS(O)₂R¹², or is represented by

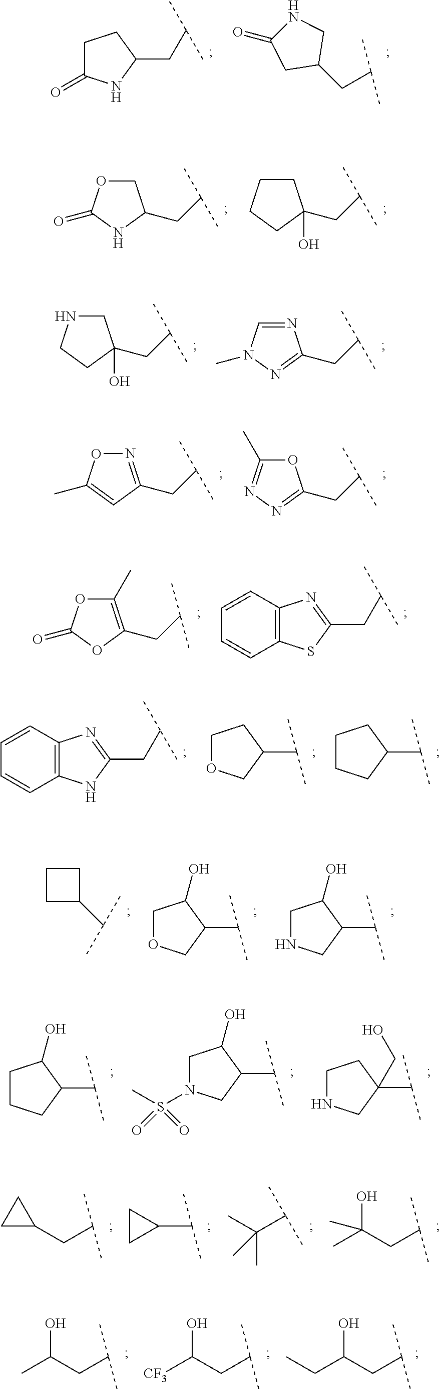

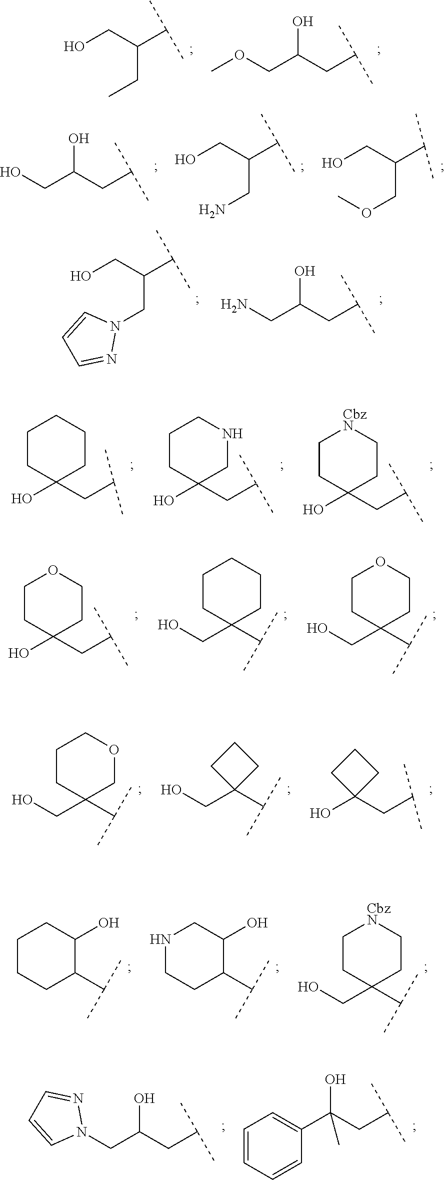

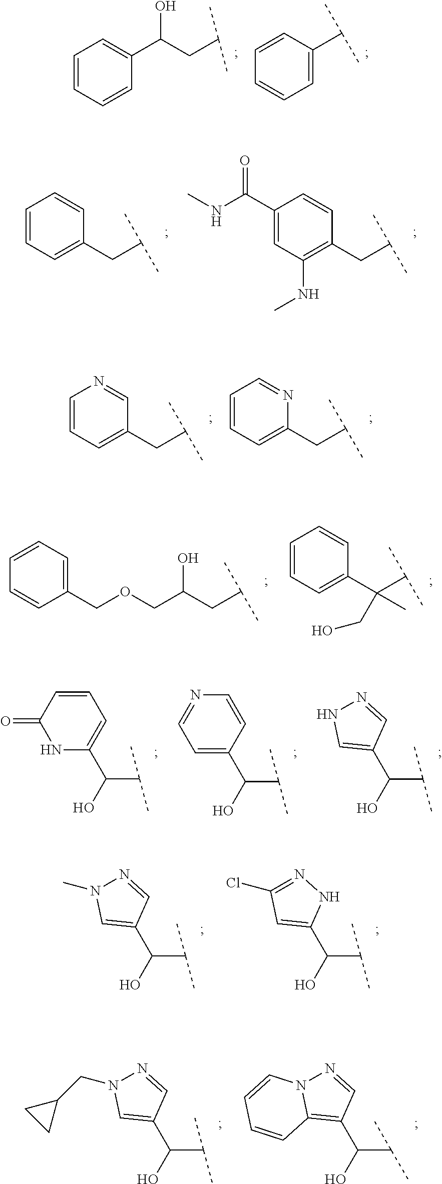

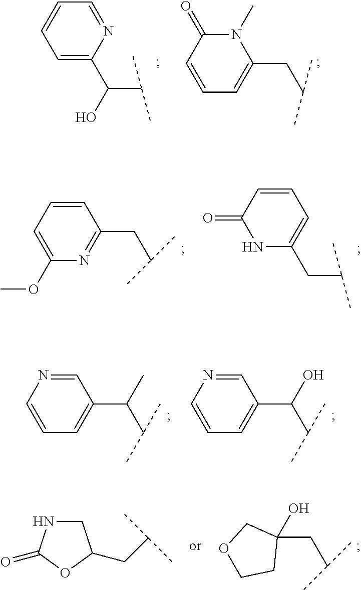

each R¹² is independently H or Me, or is represented by

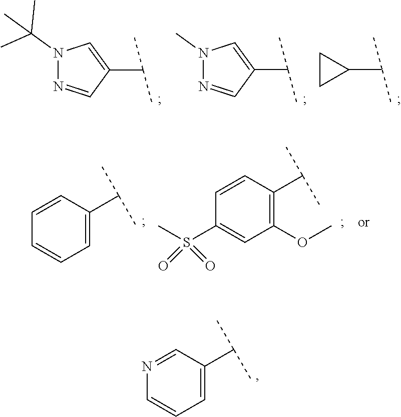

wherein R² and R⁴ are taken together with their intervening atom to form a ring represented by:

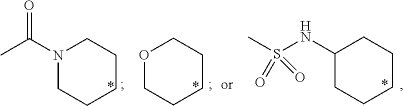

wherein “*” indicates the intervening atom between R² and R⁴; and
R⁴ is H or Me;
the remainder of variables in Formulae I, II, III, IV, and XI, are as defined in the first, eighth, ninth, tenth, eleventh, twelfth, thirteenth, fourteenth, fifteenth, sixteenth, seventeenth, eighteenth, nineteenth, and twentieth embodiments.

Twenty-fifth embodiment: a compound represented by Formulae I, II, III, IV, or XI, or a pharmaceutically acceptable salt thereof, wherein R¹ is a 4 to 8 membered monocyclic heterocyclyl, a 6 to 9 membered fused bicyclic heterocyclyl or a 6 to 9 membered bridged bicyclic heterocyclyl, each heterocyclyl optionally substituted with one or more groups selected from C₁₋₄ alkyl, C₁₋₄ haloalkyl, halo, OR^{a}, NR^{a}R^{b} or a 4-6 membered heterocyclyl containing one ring oxygen atom; or is represented by:

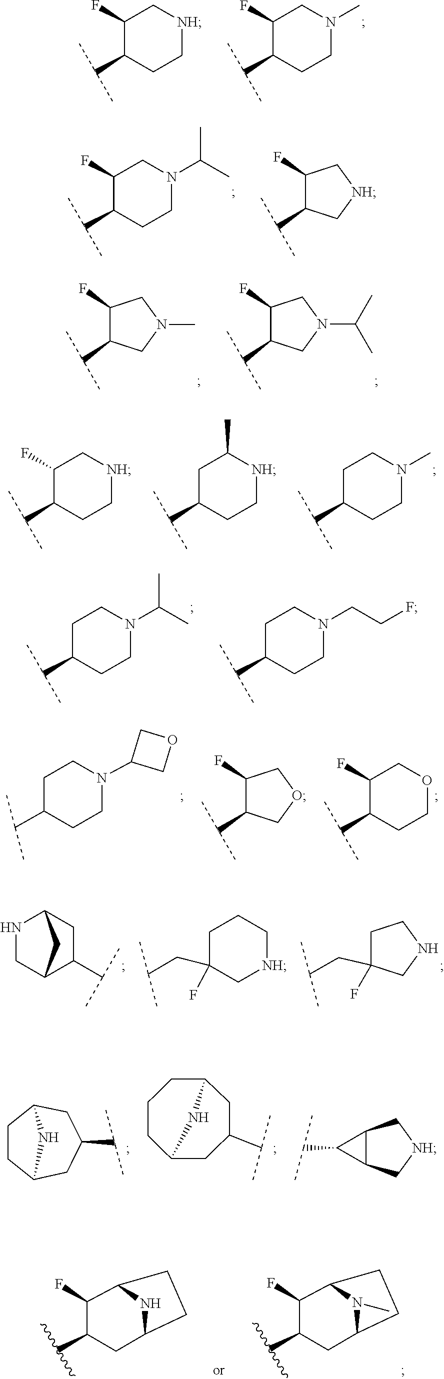

the remainder of variables in Formulae I, II, III, IV, and XI, are as defined in the first, eighth, ninth, tenth, eleventh, twelfth, thirteenth, fourteenth, fifteenth, sixteenth, seventeenth, eighteenth, nineteenth, twentieth, twenty-first, twenty-second, twenty-third, and twenty-fourth embodiments.
Twenty-sixth embodiment: a compound represented by Formulae I, II, III, IV, or XI, or a pharmaceutically acceptable salt thereof, wherein R¹ is a 4 to 7 membered monocyclic heterocyclyl containing one nitrogen optionally substituted with C₁₋₄ alkyl or fluoro, wherein the definitions of the other variables in Formulae I, II, III, IV, and XI, are as defined in the first, eighth, ninth, tenth, eleventh, twelfth, thirteenth, fourteenth, fifteenth, sixteenth, seventeenth, eighteenth, nineteenth, twentieth, twenty-first, twenty-second, twenty-third, and twenty-fourth embodiments.
Twenty-seventh embodiment: a compound represented by Formulae I, II, III, IV, or XI, or a pharmaceutically acceptable salt thereof, wherein R¹ is represented by:

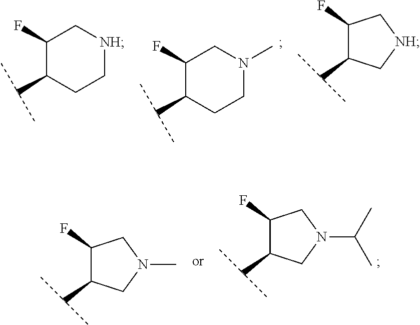

wherein the definitions of the other variables in Formulae I, II, III, IV, and XI, are as defined in the first, eighth, ninth, tenth, eleventh, twelfth, thirteenth, fourteenth, fifteenth, sixteenth, seventeenth, eighteenth, nineteenth, twentieth, twenty-first, twenty-second, twenty-third, and twenty-fourth embodiments.
Twenty-eighth embodiment: a compound represented by Formulae I, II, III, IV, or XI, or a pharmaceutically acceptable salt thereof, wherein
R^{a} is H, Me, or benzyl;
R^{b} is H or Me; and
R^{c} is H or Me;
R^{d} is H;
the remainder of variables in Formulae I, II, III, IV, and XI, are as defined in the first, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, and twenty-seventh embodiments.
Twenty-ninth embodiment: a compound represented by Formula I, or a pharmaceutically acceptable salt thereof, wherein
Y is NH;
when Z is NR^{3a}, R³ is H and R^{3a} is CH₂CF₃ or SCF₃;
when Z is S, R³ is CH₂CF₃ or SCF₃;
R⁵ is H;
R² is Me, (CH₂)ₘC(O)N(R¹²)₂, or is represented by

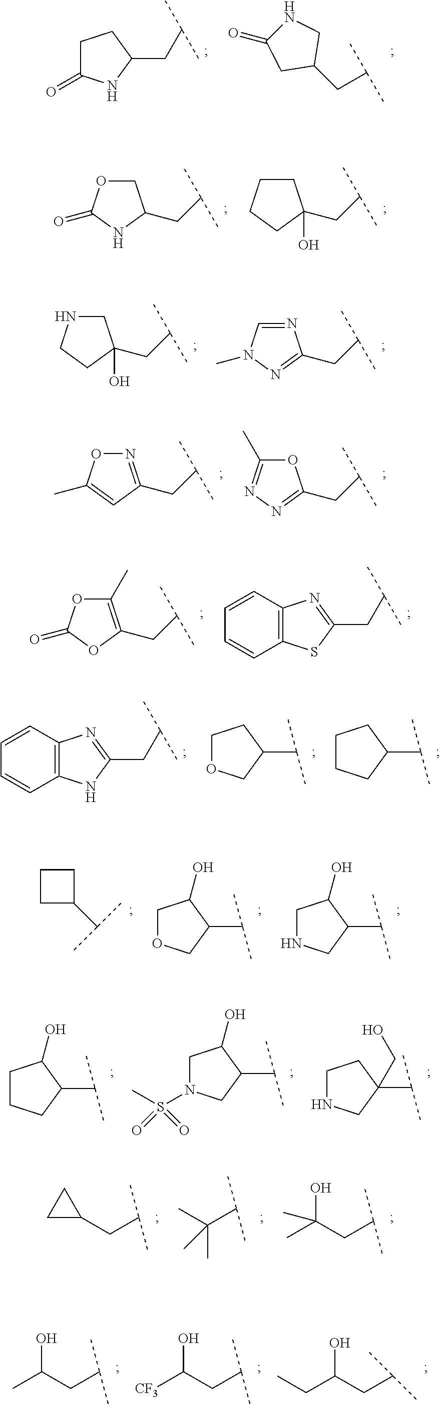

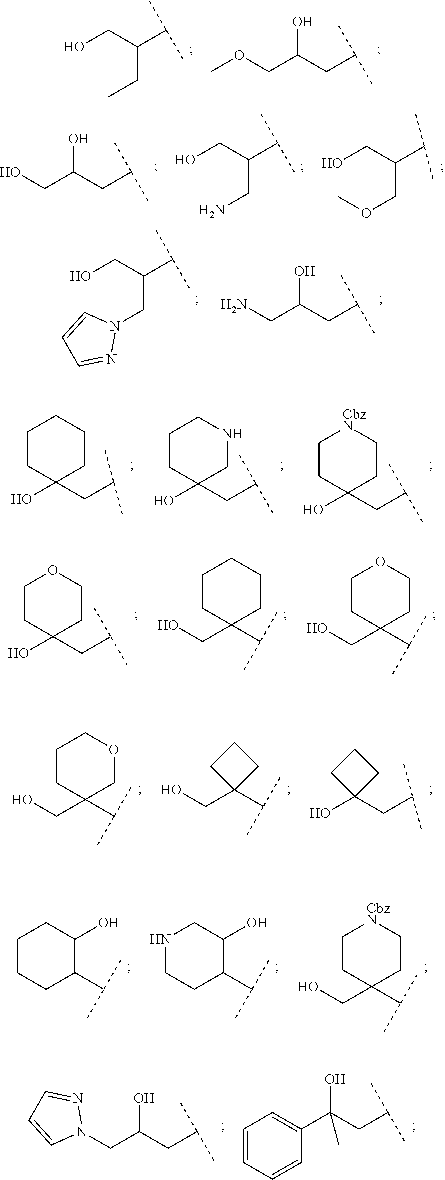

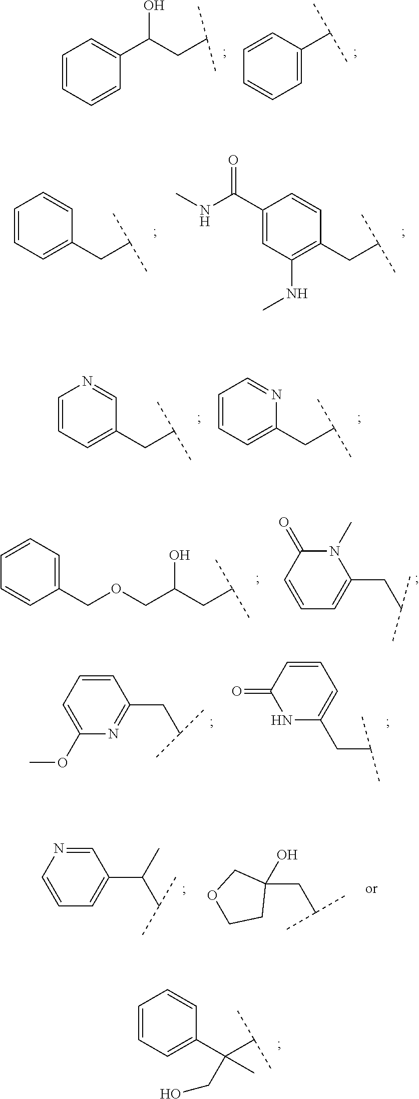

each R¹² is independently H, Me, or phenyl;
R¹ is represented by:

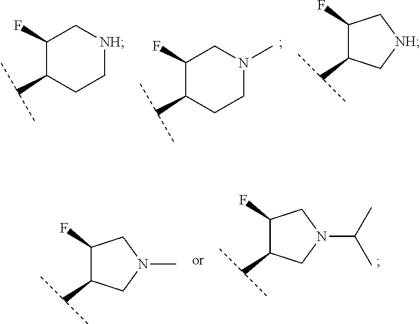

and
R^{a} is H;
the remainder of variables in Formula I are as defined in the first and ninth embodiments.

The disclosure also includes the compounds prepared in the Exemplification, in both the neutral form and pharmaceutically acceptable salts thereof. The synthetic protocol used to prepare the disclosed compounds is described in the Exemplification.

Another embodiment of the disclosure is a compound disclosed herein, including a compound of any one of Formulae I-XI, or as disclosed in the Exemplification, or a pharmaceutically acceptable salt of any of the foregoing, in which one or more hydrogen atoms is replaced with deuterium. The deuterium enrichment at any one of the sites where hydrogen has been replaced by deuterium is at least 50%, 75%, 85%, 90%, 95%, 98% or 99%. Deuterium enrichment is a mole percent and is obtained by dividing the number of compounds with deuterium enrichment at the site of enrichment with the number of compounds having hydrogen or deuterium at the site of enrichment.

The number of carbon atoms in a group is specified herein by the prefix “Cₓ₋ₓₓ”, or by “(Cₓ-Cₓₓ)” wherein x and xx are integers. For example, “C₃₋₆ cycloalkyl” is a cycloalkyl group which has from 3 to 6 carbon atoms, and (C₁-C₄)alkyl is an alkyl group which has from 1 to 3 carbon atoms.

The suffix “yl” added to the end of a chemical name indicates that the named moiety is bonded to the molecule at one point, i.e., monovalent.

“Alkyl”, when used alone or part of a larger moiety, refers to a fully saturated branched or unbranched hydrocarbon moiety. Unless otherwise specified, an alkyl comprises 1 to 6 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, or n-hexyl.

“Alkenyl” refers to a branched or unbranched hydrocarbon moiety containing at least one double bond. Unless otherwise specified, an alkenyl group comprises 2 to 6 carbon atoms, or 2 to 4 carbon atoms. Representative examples of alkenyl include, but are not limited to, ethenyl, propenyl, 1-butenyl, 2-butenyl, 1-methypropenyl, 2-methypropenyl, 3-methypropenyl and the like.

“Alkynyl” refers to a branched or unbranched hydrocarbon moiety containing at least one triple bond. Unless otherwise specified, an alkynyl group comprises 2 to 6 carbon atoms, or 2 to 4 carbon atoms. Representative examples of alkynyl include, but are not limited to, ethynyl, propynyl, 1-butynyl, 2-butynyl, 1-methypropynyl, 2-methypropynyl, 3-methypropynyl and the like.

“Alkoxy” refers to OR, where oxygen is singularly bonded to R, and R is an alkyl group. Examples of alkoxy include methoxy, ethoxy, isopropoxy, and the like.

“Aryl”, when used alone or as part of another moiety such as aralkyl, refers to an aromatic hydrocarbon of six to 10 ring atoms, such as phenyl or naphthyl.

“Halogen” or “halo” is fluoro, chloro, bromo or iodo.

“Cycloalkyl” refers to completely saturated monocyclic or bicyclic hydrocarbon group. Unless otherwise specified, a cycloalkyl has 3-10 ring carbon atoms, alternatively 3-8 ring carbon atoms. A cycloalkyl can be monocyclic, or fused bicyclic. A monocyclic cycloalkyl has 3-8 ring carbon atoms and includes cyclopropyl, cyclobutyl, cyclopentyl, cyclohexyl, cyclopheptyl and cyclooctyl. A fused bicyclic cycloalkyl has 8-10 ring carbon atoms and two rings which share two adjacent ring atoms, e.g., a 4 to 7 membered cycloalkyl fused to a 3 to 6 membered cycloalkyl. Examples include decahydronapthalene, octahydro-1H-indene, octahydropentalene, decahydroazulene, decahydro-1H-annulene, bicycle[4.2.0]octane, bicycle[3.2.0]heptane, and the like.

“Heteroaryl” refers to an aromatic 5- to 10-membered mono or bicyclic cyclic ring system, having 1 to 4 heteroatoms independently selected from O, N and S, and wherein N can be oxidized (e.g., N(O)) or quaternized, and S can be optionally oxidized to sulfoxide and sulfone. A monocyclic heteroaryl has 5 or 6 ring atoms, i.e., is 5 to 6 membered. Examples of 5- to 6-membered monocyclic heteroaryls include, but are not limited to, pyrazolyl, imidazolyl, pyridyl, pyrimidyl, oxazolyl, oxadiazolyl, isoxazolyl, thiazolyl, triazolyl, tetrazolyl, and the like.

A bicyclic heteroaryl has 8 to 10 ring atoms, i.e., is 8 to 10 membered. Examples of 8- to 10-membered bicyclic heteroaryls include, but are not limited to pyrazolopyridyl, indolyl, indazolyl, azaindolyl, benzimidazolyl, benzoxazolyl, benzothiazolyl, benzofuranyl, benzothiofuranyl, quinolinyl, isoquinolinyl and the like.

“Heterocyclyl” refers to a saturated or partially unsaturated monocyclic or bicyclic (e.g., fused or bridged) ring system which has, unless otherwise specified, from 4- to 12-ring members, e.g., 4-10 ring members, at least one of which is a heteroatom, and up to 4 (e.g., 1, 2, 3, or 4) of which may be heteroatoms, wherein the heteroatoms are independently selected from O, S and N.

An “oxygen-containing heterocyclyl” is a heterocyclyl comprising a ring oxygen atom. An oxygen-containing heterocyclyl can have more than one ring heteroatom. A “nitrogen-containing heterocyclyl” is a heterocyclyl comprising a ring nitrogen atom. A nitrogen-containing heterocyclyl can have more than one ring heteroatom.

Examples of 4-7 membered monocyclic heterocyclyl include, but are not limited to, oxetanyl, thietanyl, azetedinyl, pyrrolidinyl, dihydropyridinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, trioxanyl, trithianyl, azepanyl, oxepanyl, thiepanyl, dihydrofuranyl, imidazolinyl, and dihydropyranyl.

A “fused bicyclic heterocyclyl” has a 4-7 membered heterocyclyl which shares two adjacent ring atoms with a 4-7 membered heterocyclyl or a 3-7 membered cycloalkyl, i.e., a 4 to 7 membered heterocyclyl fused to a 4 to 7 membered heterocyclyl or a 3 to 7 membered carbocyclyl. Examples include cyclopropylpyrrolidinyl, cyclopentapyrrolidinyl, cyclopentapiperidinyl, cyclopentaazapanyl, cyclohexapyrrolidinyl, cyclohexapiperidinyl, cyclohexaazapanyl, cycloheptapyrrolidinyl, cycloheptapiperidinyl, cycloheptaazapanyl, pyranopyrrolidinyl, pyranopiperidinyl, pyranoazapanyl, and the like.

A “bridged bicyclic heterocyclyl” has 7-11 members and comprises a 5 to 7 membered heterocyclyl which shares three ring atoms with a 5 to 7 membered heterocyclyl or a 5 to 7 membered non-aromatic cycloalkyl. Examples of nitrogen containing bridged bicyclics include azabicyclo[2.2.1]hepantyl, azabicyclo[3.2.1]octanyl, azabicyclo[3.3.1]nonanyl, diazabicyclo[2.2.1]hepantyl, diazabicyclo[3.2.1]octanyl and diazabicyclo[3.3.1]nonanyl. Examples of oxygen containing bridged bicyclics include oxobicyclo[2.2.1]hepantyl, oxobicyclo[3.2.1]octanyl, oxobicyclo[3.3.1]nonanyl, oxa-azabicyclo[2.2.1]hepantyl, oxa-azabicyclo[3.2.1]octanyl and oxa-azabicyclo[3.3.1]nonanyl.

The term “substituted”, whether preceded by the term “optionally” or not, refers to the replacement of a hydrogen substituent in a given structure with a non-hydrogen substituent. Thus, for example, a substituted alkyl is an alkyl wherein at least one non-hydrogen substituent is in the place of a hydrogen substituent on the alkyl group. To illustrate, monofluoroalkyl is an alkyl substituted with a fluoro substituent, and difluoroalkyl is an alkyl substituted with two fluoro substituents. It should be recognized that if there is more than one substitution on a substituent, each non-hydrogen substituent can be identical or different (unless otherwise stated).

If a group is described as “optionally substituted”, the group can be either (1) not substituted or (2) substituted. If a group is described as optionally substituted with up to a particular number of non-hydrogen substituents, that group can be either (1) not substituted; or (2) substituted by up to that particular number of non-hydrogen substituents or by up to the maximum number of substitutable positions on the substituent, whichever is less. Thus, for example, if a group is described as a cycloalkyl optionally substituted with up to 3 non-hydrogen substituents, then any cycloalkyl with less than 3 substitutable positions would be optionally substituted by up to only as many non-hydrogen substituents as the cycloalkyl has substitutable positions.

Spirocycloalkyl or spiroheterocyclyl refers to a cycloalkyl or heterocyclyl that shares one ring atom with another group, such as another cycloalkyl or heterocyclyl.

Compounds having one or more alkene can exist in various stereoisomeric forms, i.e., each alkene can have a Z or E configuration, or can be a mixture of both. Stereoisomers are compounds that differ only in their spatial arrangement. Stereoisomers include both Z and E forms of an alkene. Z alkenes are arranged such that the highest priority functional groups (IUPAC rules) on adjacent alkene carbon atoms are on the same side of the carbon-carbon double bond. E alkenes are arranged such that the highest priority functional groups on adjacent alkene carbon atoms are on opposite sides of the carbon-carbon double bond.

An “oxime” compound contains a carbon-nitrogen double bond, wherein said nitrogen is also attached to oxygen through a single bond. Compounds having one or more oxime can exist in various stereoisomeric forms, i.e., each oxime can have a Z or E configuration, or can be a mixture of both. Stereoisomers are compounds that differ only in their spatial arrangement. Stereoisomers include both Z and E forms of an oxime. Z oximes are arranged such that both the highest priority functional group on the carbon (IUPAC rules), and the oxygen bound to nitrogen are on the same side of the carbon-nitrogen double bond. E oximes are arranged such that both the highest priority functional group on the carbon, and the oxygen bound to nitrogen are on opposite sides of the carbon-nitrogen double bond.

When the stereochemical configuration in a compound having one or more alkene or oxime is depicted by its chemical name (e.g., where the configuration is indicated in the chemical name by “Z” or “E”) or by its drawn structure, the enrichment of the indicated configuration relative to the opposite configuration is greater than 50%, 60%, 70%, 80%, 90%, 99% or 99.9% “Enrichment of the indicated configuration relative to the opposite configuration” is a mole percent and is determined by dividing the number of compounds with the indicated stereochemical configuration by the total number of all of the compounds with the same or opposite stereochemical configuration in a mixture.

Compounds having one or more chiral centers can exist in various stereoisomeric forms, i.e., each chiral center can have an R or S configuration or can be a mixture of both. Stereoisomers are compounds that differ only in their spatial arrangement. Stereoisomers include all diastereomeric and enantiomeric forms of a compound. Enantiomers are stereoisomers that are non-superimposable mirror images of each other. Diastereomers are stereoisomers having two or more chiral centers that are not identical and are not mirror images of each other.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9% “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9% (except when the designation “rac” or “racemate accompanies the structure or name, as explained in the following two paragraphs). “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that, unless otherwise indicated, one of the encompassed stereoisomers or any mixture of the encompassed stereoisomers are included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

In cases where a compound provided herein is sufficiently basic or acidic to form stable nontoxic acid or base salts, preparation and administration of the compounds as pharmaceutically acceptable salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, a ketoglutarate, or α-glycerophosphate. Inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Pharmaceutically-acceptable base addition salts can be prepared from inorganic and organic bases. Salts from inorganic bases, can include but are not limited to, sodium, potassium, lithium, ammonium, calcium or magnesium salts. Salts derived from organic bases can include, but are not limited to, salts of primary, secondary or tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocycloalkyl amines, diheterocycloalkyl amines, triheterocycloalkyl amines, or mixed di- and tri-amines where at least two of the substituents on the amine can be different and can be alkyl, substituted alkyl, alkenyl, substituted alkenyl, cycloalkyl, substituted cycloalkyl, cycloalkenyl, substituted cycloalkenyl, aryl, heteroaryl, or heterocycloalkyl and the like. Also included are amines where the two or three substituents, together with the amino nitrogen, form a heterocycloalkyl or heteroaryl group. Non-limiting examples of amines can include, isopropylamine, trimethyl amine, diethyl amine, tri(iso-propyl) amine, tri(n-propyl) amine, ethanolamine, 2-dimethyl-aminoethanol, trimethamine, lysine, arginine, histidine, caffeine, procaine, hydrabamine, choline, betaine, ethylenediamine, glucosamine, N-alkylglucamines, theobromine, purines, piperazine, piperidine, morpholine, or N-ethylpiperidine, and the like. Other carboxylic acid derivatives can be useful, for example, carboxylic acid amides, including carboxamides, lower alkyl carboxamides, or dialkyl carboxamides, and the like.

The compounds disclosed herein or pharmaceutically acceptable salts thereof can be used for reactivating mutated p53 in a subject in need thereof. The method comprises administering to the subject an effective amount of a compound disclosed herein or pharmaceutically acceptable salts thereof or a pharmaceutical composition disclosed herein. “Reactivating mutated p53” refers to increasing the ability of a mutated p53 protein to bind to DNA at physiologically relevant temperatures, where that mutated p53 protein has decreased ability to to DNA at physiologically relevant temperatures compared with wild type p53 protein. A subject in need of inhibition of p53 includes, for example, a subject with a cancer characterized by dysfunctional p53. A dysfunctional p53 includes, for example, p53 with an inactivating mutation and/or mutated p53 with decreased ability to bind to DNA at physiologically relevant temperatures compared with wild type p53 protein. Inactivating p53 mutations included Val143, His168, Arg175, Tyr220, Gly245, Arg248, Arg249, Phe270, Arg273, Arg282, and/or a combination thereof. Alternatively, the p53 mutation is V157F, R175H, Y220C, G245S, R248Q, R248W, R249S, R273H, R273C, R282W, and/or a combination thereof. In another alternative, the p53 mutation is Y220C.

Cancers which can be treated with the disclosed compounds or pharmaceutically acceptable salts thereof or pharmaceutically acceptable salts thereof or the disclosed pharmaceutical compositions include acute lymphoblastic leukemia, acute myeloid leukemia, adrenocortical carcinoma, AIDS-related cancers, AIDS-related lymphoma, anal cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, central nervous system lymphoma, cerebellar astrocytoma, cervical cancer, colon cancer, gallbladder cancer, gastric cancer, head and neck cancer, heart cancer, hepatocellular (liver) cancer, kidney cancer, liver cancer, lung cancers, such as non-small cell and small cell lung cancer, ovarian cancer, ovarian epithelial cancer, ovarian germ cell tumor, pancreatic cancer, pancreatic cancer islet cell, prostate cancer, rectal cancer, renal cell carcinoma, skin cancers, skin carcinoma merkel cell, small intestine cancer or throat cancer.

In one aspect, the disclosed compounds or pharmaceutically acceptable salts thereof or disclosed pharmaceutical compositions can be part of a combination therapies with one or more other therapeutic agents.

In some embodiments, one or more other therapeutic agents can be an immune checkpoint inhibitor. In some embodiments, the checkpoint inhibitors include but are not limited to anti programed cell death receptor-1 (aPD-1) monoclonal antibodies such as pembrolizumab, nivolumab, cemiplimab, or anti programed cell death receptor-1 ligand (aPD-L1) monoclonal antibodies such as atezolizumab, dostarlimab, durvalumab and avelumab, or anti cytotoxic T lymphocyte-associated antigen (anti-CTLA4) monoclonal antibodies such as ipilimumab and tremelimumab, or anti lymphocyte activated gene-3 (LAG-3) monoclonal antibodies such as relatlimab.

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL).

In some embodiments, one or more other therapeutic agent is an inhibitor of interaction between p53 and MDM2. Said MDM2 inhibitors include but are not limited to navtemadlin (AMG-232, KRG 232, Amgen), idasanutlin (RG7388, Hoffman-La Roche), milademetan (RAIN-32), MK-8242 (Merck), SAR405838, NVP-CGM097, RG7112, and DS-3032b.

In some embodiments, one or more other therapeutic agent is an MDM2 targeted protein degrader such as MD-224.

In some embodiments, one or more other therapeutic agent is a Poly ADP ribose polymerase (PARP) inhibitor. In some embodiments, a PARP inhibitor is selected from olaparib (LYNPARZA®, AstraZeneca); rucaparib (RUBRACA®, Clovis Oncology); niraparib (ZEJULA®, Tesaro); talazoparib (MDV3800/BMN 673/LT00673, Medivation/Pfizer/Biomarin); veliparib (ABT-888, Abb Vic); and BGB-290 (BeiGene, Inc.).

In some embodiments, one or more other therapeutic agent is a CDK inhibitor such as a CDK4/CDK6 inhibitor. In some embodiments, a CDK 4/6 inhibitor is selected from Palbociclib (IBRANCE®, Pfizer); ribociclib (KISQALI®, Novartis); abemaciclib (Ly2835219, Eli Lilly); and trilaciclib (G1T28, G1 Therapeutics). In some embodiments, a CDK inhibitor is a CDK9 selective inhibitor selected from dinaciclib, AT-7519, P276-00, AZD-4573, alvocidib/flavopiridol, CYC065, atuveciclib, BAY-1251152, voruciclib or GFH009.

In some embodiments, one or more other therapeutic agent is an inhibitor of antiapoptotic proteins, such as BCL-2. Approved anti-apoptotics which may be used in the present invention include venetoclax (VENCLEXTA®, AbbVie/Genentech); and blinatumomab (BLINCYTO®, Amgen). Other therapeutic agents targeting apoptotic proteins which have undergone clinical testing and may be used in the present invention include navitoclax (ABT-263, Abbott). Other therapeutic agents targeting BCL family proteins via E3 ligase-mediated target protein degradation may be used in the present invention.

In some embodiments, one or more other therapeutic agent is a platinum-based therapeutic, also referred to as platins. Platins cause cross-linking of DNA, such that they inhibit DNA repair and/or DNA synthesis, mostly in rapidly reproducing cells, such as cancer cells.

In some embodiments, a platinum-based therapeutic is selected from cisplatin (PLATINOL®, Bristol-Myers Squibb); carboplatin (PARAPLATIN®, Bristol-Myers Squibb; also, Teva; Pfizer); oxaliplatin (ELOXITIN® Sanofi-Aventis); nedaplatin (AQUPLA®, Shionogi), picoplatin (Poniard Pharmaceuticals); and satraplatin (JM-216, Agennix).

In some embodiments, one or more other therapeutic agent is a taxane compound, which causes disruption of microtubules, which are essential for cell division. In some embodiments, a taxane compound is selected from paclitaxel (TAXOL®, Bristol-Myers Squibb), docetaxel (TAXOTERE®, Sanofi-Aventis; DOCEFREZ®, Sun Pharmaceutical), albumin-bound paclitaxel (ABRAXANE®; Abraxis/Celgene), cabazitaxel (JEVTANA®, Sanofi-Aventis), and SID530 (SK Chemicals, Co.).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, YONDELIS®, Janssen Oncology), mechlorethamine (alkylating agent, VALCHLOR®, Aktelion Pharmaceuticals); vincristine (ONCOVIN®, Eli Lilly; VINCASAR®, Teva Pharmaceuticals; MARQIBO®, Talon Therapeutics); temozolomide (prodrug to alkylating agent 5-(3-methyltriazen-1-yl)-imidazole-4-carboxamide (MTIC) TEMODAR®, Merck); cytarabine injection (ara-C, antimetabolic cytidine analog, Pfizer); lomustine (alkylating agent, CEENU®, Bristol-Myers Squibb; GLEOSTINE®, NextSource Biotechnology); azacytidine (pyrimidine nucleoside analog of cytidine, VIDAZA®, Celgene); omacetaxine mepesuccinate. (cephalotaxine ester) (protein synthesis inhibitor, SYNRIBO®; Teva Pharmaceuticals); asparaginase Erwinia chrysanthemi (enzyme for depletion of asparagine, ELSPAR®, Lundbeck; ERWINAZE®, EEISA Pharma); eribulin mesylate (microtubule inhibitor, tubulin-based antimitotic, HALAVEN®, Eisai); cabazitaxel (microtubule inhibitor, tubulin-based antimitotic, JEVTANA®, Sanofi-Aventis); capacetrine (thymidylate synthase inhibitor, XELODA®, Genentech); bendamustine (bifunctional mechlorethamine derivative, believed to form interstrand DNA cross-links, TREANDA®, Cephalon/Teva); ixabepilone (semi-synthetic analog of epothilone B, microtubule inhibitor, tubulin-based antimitotic, IXEMPRA®, Bristol-Myers Squibb); nelarabine (prodrug of deoxyguanosine analog, nucleoside metabolic inhibitor, ARRANON®, Novartis); clorafabine (prodrug of ribonucleotide reductase inhibitor, competitive inhibitor of deoxycytidine, CLOLAR®, Sanofi-Aventis); and trifluridine and tipiracil (thymidinebased nucleoside analog and thymidine phosphorylase inhibitor, LONSEIRF®, Taiho Oncology).

In some embodiments, one or more other therapeutic agent is a phosphatidylinositol 3 kinase (PI3K) inhibitor. In some embodiments, a PBK inhibitor is selected from idelalisib (ZYDELIG®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In some embodiments, one or more other therapeutic agent is a kinase inhibitor or VEGF-R antagonist. Approved VEGF inhibitors and kinase inhibitors useful in the present invention include: bevacizumab (AVASTIN®, Genentech/Roche) an anti-VEGF monoclonal antibody; ramucirumab (CYRAMZA®, Eli Lilly), an anti-VEGFR-2 antibody and ziv-aflibercept, also known as VEGF Trap (ZALTRAP®; Regeneron/Sanofi). VEGFR inhibitors, such as regorafenib (STIVARGA®, Bayer); vandetanib (CAPRELSA®, AstraZeneca); axitinib (INLYTA®, Pfizer); and lenvatinib (LENVIMA®, Eisai); Raf inhibitors, such as sorafenib (NEXAVAR®, Bayer AG and Onyx); dabrafenib (TAFINLAR®, Novartis); and vemurafenib (ZELBORAF®, Genentech/Roche); MEK inhibitors, such as cobimetanib (COTELLIC®, Exelexis/Genentech/Roche); trametinib (MEKINIST®, Novartis); Bcr-Abl tyrosine kinase inhibitors, such as imatinib (GLEEVEC®, Novartis); nilotinib (TASIGNA®, Novartis); dasatinib (SPRYCEL®, BristolMyersSquibb); bosutinib (BOSULIF®, Pfizer); and ponatinib (INCLUSIG®, Ariad Pharmaceuticals); Her2 and EGFR inhibitors, such as gefitinib (IRESSA®, AstraZeneca); erlotinib (TARCEEVA®, Genentech/Roche/Astellas); lapatinib (TYKERB®, Novartis); afatinib (GILOTRIF®, Bochringer Ingelheim); osimertinib (targeting activated EGFR, TAGRISSO®, AstraZeneca); and brigatinib (ALUNBRIG®, Ariad Pharmaceuticals); c-Met and VEGFR2 inhibitors, such as cabozanitib (COMETRIQ®, Excelexis); and multikinase inhibitors, such as sunitinib (SUTENT®, Pfizer); pazopanib (VOTRIENT®, Novartis); ALK inhibitors, such as crizotinib (XALKORI®, Pfizer); ceritinib (ZYKADIA®, Novartis); and alectinib (ALECENZa®, Genentech/Roche); Bruton's tyrosine kinase inhibitors, such as ibrutinib (IMBRErVICA®, Pharmacyclics/Janssen); and Flt3 receptor inhibitors, such as midostaurin (RYE) APT®, Novartis).

Other kinase inhibitors and VEGF-R antagonists that are in development and may be used in the present invention include tivozanib (Aveo Pharmaceuticals); vatalanib (Bayer/Novartis); lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (SUPECT®, IY5511, Il-Yang Pharmaceuticals, S. Korea); ruxolitinib (JAKAFI®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/T akeda).

In some embodiments, one or more other therapeutic agent is an mTOR inhibitor, which inhibits cell proliferation, angiogenesis and glucose uptake. In some embodiments, an mTOR inhibitor is everolimus (AFINITOR®, Novartis); temsirolimus (TORISEL®, Pfizer); and sirolimus (RAPAMUNE®, Pfizer).

In some embodiments, one or more other therapeutic agent is a proteasome inhibitor. Approved proteasome inhibitors useful in the present invention include bortezomib (VELCADE®, Takeda); carfilzomib (KYPROLIS®, Amgen); and ixazomib (NINLARO®, Takeda).

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (LARTRUVO®; Eli Lilly). Approved EGFR antagonists which may be used in the present invention include cetuximab (ERBITUX®, Eli Lilly); necitumumab (PORTRAZZA®, Eli Lilly), panitumumab (VECTIBIX®, Amgen); and Osimertinib (targeting activated EGFR, TAGRISSO®, AstraZeneca).

In some embodiments, one or more other therapeutic agent is an aromatase inhibitor. In some embodiments, an aromatase inhibitor is selected from exemestane (AROMASIN®, Pfizer); anastazole (ARIMIDEX®, AstraZeneca) and letrozole (FEMARA®, Novartis).

In some embodiments, one or more other therapeutic agent is a folic acid inhibitor. Approved folic acid inhibitors useful in the present invention include pemetrexed (ALIMTA®, Eli Lilly).

In some embodiments, one or more other therapeutic agent is a topoisomerase inhibitor. Approved topoisomerase inhibitors useful in the present invention include irinotecan (ONIVYDE®, Merrimack Pharmaceuticals); topotecan (HYCAMTIN®, GlaxoSmithKline). Topoisomerase inhibitors being studied which may be used in the present invention include pixantrone (PIXUVRI®, CTI Biopharma).

Pharmaceutical compositions are disclosed that include one or more compounds provided herein or a pharmaceutically acceptable salt thereof, and typically at least one additional substance, such as an excipient, a known therapeutic other than those of the disclosure, and combinations thereof. In some embodiments, the disclosed compounds or pharmaceutically acceptable salts thereof can be used in combination with other agents known to have beneficial activity targeting diseases or disorders listed above. For example, disclosed compounds or pharmaceutically acceptable salts thereof can be administered alone or in combination with one or more anti-cancer or antiviral agent.

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

A “subject” is a mammal in need of medical treatment, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The precise amount of compound or pharmaceutically acceptable salt thereof administered to provide an “effective amount” to the subject will depend on the mode of administration, the type, and severity of the disease or condition, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer or antiviral agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the disclosure or a pharmaceutically acceptable salt thereof being used by following, for example, dosages reported in the literature and recommended in the Physician's Desk Reference (57th ed., 2003).

The term “effective amount” means an amount when administered to the subject which results in beneficial or desired results, including clinical results, e.g., inhibits, suppresses or reduces the symptoms of the condition being treated in the subject as compared to a control. For example, an effective amount can be given in unit dosage form (e.g., 0.1 mg to about 50 g per day).

The particular mode of administration and the dosage regimen will be selected by the attending clinician, taking into account the particulars of the case (e.g. the subject, the disease, the disease state involved, the particular treatment, and whether the treatment is prophylactic). Treatment can involve daily or multi-daily or less than daily (such as weekly or monthly etc.) doses over a period of a few days to months, or even years.

The pharmaceutical composition of the disclosure is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

“Pharmaceutically acceptable excipient” and “pharmaceutically acceptable carrier” refer to a substance that aids the formulation and/or administration of an active agent to and/or absorption by a subject and can be included in the compositions of the disclosure without causing a significant adverse toxicological effect on the subject. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds.

The following examples are intended to be illustrative and are not meant in any way to be limiting.

### EXEMPLIFICATION

Unless otherwise specified, abbreviations used herein will have the meaning as commonly used in the art, some of which are provided below:
AcOH—acetic acid
Aq.—Aqueous
ACN—Acetonitrile
Boc₂O—Di-tert-butyl dicarbonate
br s—Broad singlet
n-BuLi—n-Butyllithium
° C.—degree Celsius
Cbz—Benzyloxycarbonyl
CDCl₃—Deuterated chloroform
Cs₂CO₃—Cesium carbonate
d—Doublet
dd—Doublet of doublet
δ—Delta
DCM—Dichloromethane
DIAD—Disopropyl azodicarboxylate
DIEA—Diisopropylethylamine
DIPA—N,N-diisopropylamine
DMF—N,N-Dimethylformamide
DMSO-d₆—Deuterated dimethyl sulfoxide
EDCI—1-Ethyl-3-(3-dimethylaminopropyl)carbodiimide
ESI—Electrospray ionization
EtOH—ethanol
Et₃N—Triethylamine
EA—Ethyl acetate
¹⁹F NMR—Fluorine-19 nuclear magnetic resonance
g—Gram
GCMS—Gas chromatography-mass spectrometry
h or hr—Hour
¹H—Proton
¹H NMR—Proton nuclear magnetic resonance
H₂O—Water
HCl—Hydrochloric acid
HOBt—1-Hydroxybenzotriazole
HPLC—High performance liquid chromatography
Hz—Hertz
J—Coupling constant
LCMS—Liquid chromatography mass spectrometry
M⁺—Molecular ion
m—Multiplet
MeCN—Acetonitrile
MeI—Methyl iodide
MeOH—Methanol
MeOD—deuterated methanol
mg—Milligrams
min—Minutes
MHz—Mega Hertz (frequency)
mL—Milliters
mm—Millimeters
mmol—Millimoles
MS—Mass spectroscopy
NaBH₃CN—Sodium cyanoborohydride
NH₂OH—Hydroxylamine
NHPI—N-hydroxyphthalimide
NH₄HCO₃—Ammonium bicarbonate
Sat.NaHCO₃—Saturated sodium hydrogencarbonate
Na₂SO₄—Sodium sulfate
PE—Petroleum ether
%—Percentage
pH—potential of Hydrogen
ppm—Parts per million
q—Quartet
Rₜ—Retention time
rt—room temperature
s—Singlet
t—Triplet
TBAF—Tetrabutylammonium fluoride
THF—Tetrahydrofuran
TLC—Thin layer chromatography
Prep TLC—Preparative thin layer chromatography
μL—Microliters
μm—Millimeters
umol—Micromoles
Xphos—dicyclohexyl[2′,4′,6′-tris(propan-2-yl)[1,1′-biphenyl]-2-yl]phosphane
XPhos Pd G3—(2-Dicyclohexylphosphino-2′,4′,6′-triisopropyl-1,1′-biphenyl)[2-(2′-amino-1,1′-biphenyl)]palladium(II) methanesulfonate

The following Examples, while representing preferred embodiments of the invention, serve to illustrate the invention without limiting its scope.

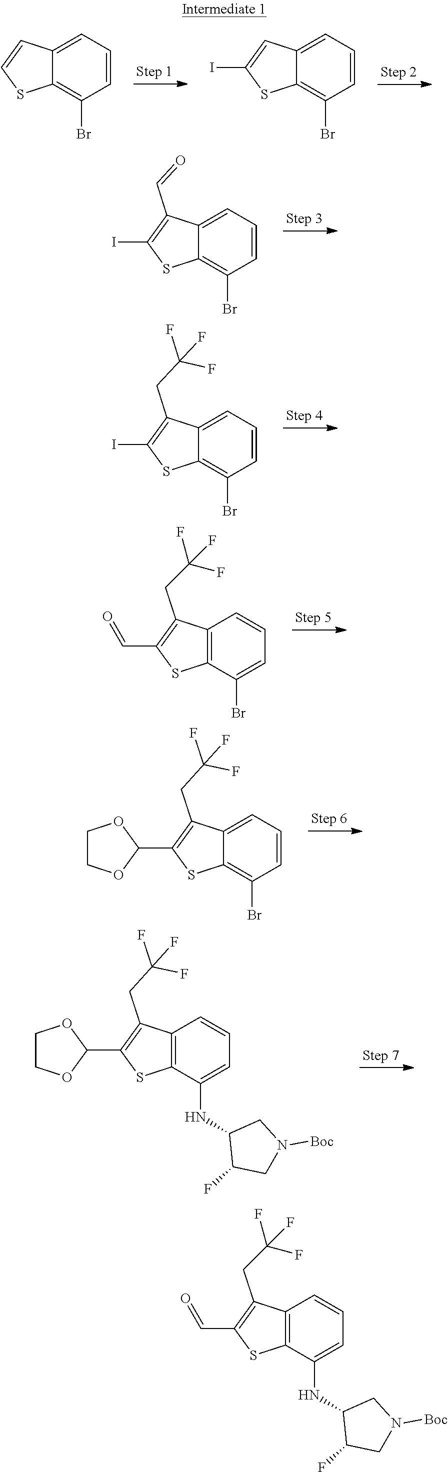

### Step 1. 7-bromo-2-iodobenzo[b]thiophene

To a stirred solution of DIPA (72.5 mL, 516.2 mmol) in THF (180 mL) was added n-BuLi (2.5 M in hexane, 206.5 mL, 516.2 mmol) dropwise at −78° C. under nitrogen atmosphere. The resulting mixture was stirred for 30 min at −78° C. under nitrogen atmosphere. To the above mixture was added solution of 7-bromo-1-benzothiophene (100 g, 469.3 mmol) in THF (360 mL) dropwise at −78° C. The resulting mixture was stirred for 30 min at −78° C. To the above mixture was added solution of I₂ (119.1 g, 469.3 mmol) in THF (210 mL) dropwise at −78° C. The resulting mixture was stirred for 30 min at −78° C. The reaction was quenched with an aq. solution of sodium sulfite (1.0 M, 100 mL) at −78° C. The resulting mixture was extracted with CH₂Cl₂ (3×1000 mL). The combined organic layers were dried over anhydrous Na₂SO₄. After filtration, the filtrate was concentrated under reduced pressure. The residue was purified by silica gel column chromatography, eluted with CH₂Cl₂/PE (1:10) to afford 7-bromo-2-iodobenzo[b]thiophene (90 g) as a solid. GCMS (M)⁺ m/z: 337.9.

### Step 2. 7-bromo-2-iodobenzo[b]thiophene-3-carbaldehyde

To a stirred solution of 7-bromo-2-iodobenzo[b]thiophene (90 g, 265.5 mmol) and dichloromethyl methyl ether (61.0 g, 531.0 mmol) in DCM (1.2 L) was added TiCl₄ (44.4 mL, 398.2 mmol) dropwise at 0° C. The resulting mixture was stirred for 2 h at rt. The reaction was quenched with water at 0° C. The resulting mixture was extracted with CH₂Cl₂ (3×500 mL). The combined organic layers were dried over anhydrous Na₂SO₄. After filtration, the filtrate was concentrated under reduced pressure. The residue was purified by silica gel column chromatography, eluted with DCM/EA (9:1) to afford 7-bromo-2-iodobenzo[b]thiophene-3-carbaldehyde (54 g) as a solid. GCMS (M)⁺ m/z: 365.8.

### Step 3. 7-bromo-2-iodo-3-(2,2,2-trifluoroethyl)benzo[b]thiophene

A solution of 7-bromo-2-iodobenzo[b]thiophene-3-carbaldehyde (54 g, 147.1 mmol) and 2,2-difluoro-2-(triphenylphosphoniumyl)acetate (104.8 g, 294.3 mmol, 2 in DMF (450 mL) was stirred for 30 min at 80° C. under nitrogen atmosphere. To the above mixture was added TBAF (1.0 M in THF, 295.0 mL, 295.0 mmol) and H₂O (0.6 mL, 33.3 mmol) at 80° C. The resulting mixture was stirred for 30 min at 80° C. The resulting mixture was diluted with water (2000 mL). The resulting mixture was extracted with EA (3×2000 mL). The combined organic layers were washed with brine, dried over anhydrous Na₂SO₄. After filtration, the filtrate was concentrated under reduced pressure. The residue was purified by silica gel column chromatography, eluted with PE to afford 7-bromo-2-iodo-3-(2,2,2-trifluoroethyl)benzo[b]thiophen (33 g) as a solid. GCMS (M)⁺ m/z: 419.9.

### Step 4. 7-bromo-3-(2,2,2-trifluoroethyl)benzo[b]thiophene-2-carbaldehyde

To a stirred solution of 7-bromo-2-iodo-3-(2,2,2-trifluoroethyl)benzo[b]thiophene (50 g, 118.8 mmol) in THF (50 mL) were added i-PrMgCl·LiCl (1.3 M in THF, 100 mL, 130 mmol) dropwise at −78° C. under nitrogen atmosphere. The resulting mixture was stirred for 5 min at −78° C. under nitrogen atmosphere. To the above mixture was added DMF (9.6 g, 130.6 mmol) dropwise over 10 min at −78° C. The resulting mixture was stirred for additional 30 min at rt. The reaction was quenched by the addition of water/ice at 0° C. The resulting mixture was extracted with EA (3×500 mL). The combined organic layers dried over anhydrous Na₂SO₄. After filtration, the filtrate was concentrated under reduced pressure. The residue was purified by silica gel column chromatography, eluted with PE/DCM (10:1) to afford 7-bromo-3-(2,2,2-trifluoroethyl)benzo[b]thiophene-2-carbaldehyde (25 g) as a solid. LCMS [M+H]⁺ m/z: 323.3.

### Step 5. 2-(7-bromo-3-(2,2,2-trifluoroethyl)benzo[b]thiophen-2-yl)-1,3-dioxolane

A mixture of 7-bromo-3-(2,2,2-trifluoroethyl)benzo[b]thiophene-2-carbaldehyde (20 g, 61.9 mmol), TsOH·H₂O (14.1 g, 74.3 mmol), 4 Å molecular sieve (20 g) and ethane-1,2-diol (5.8 g, 92.9 mmol) in toluene (200 mL) was stirred for 3 h at 110° C. under nitrogen atmosphere. The reaction mixture was basified with sat. NaHCO₃ (aq.). The resulting mixture was extracted with EA (3×300 mL). The combined organic layers were dried over anhydrous Na₂SO₄. After filtration, the filtrate was concentrated under reduced pressure. This resulted in 2-(7-bromo-3-(2,2,2-trifluoroethyl)benzo[b]thiophen-2-yl)-1,3-dioxolane (23 g) as a solid. LCMS [M+H]⁺ m/z: 366.9.

### Step 6. Tert-butyl (3R,4S)-3-((2-(1,3-dioxolan-2-yl)-3-(2,2,2-trifluoroethyl)benzo[b]thiophen-7-yl)amino)-4-fluoropyrrolidine-1-carboxylate

A mixture of 2-(7-bromo-3-(2,2,2-trifluoroethyl)benzo[b]thiophen-2-yl)-1,3-dioxolane (5 g, 13.6 mmol), tert-butyl (3R,4S)-3-amino-4-fluoropyrrolidine-1-carboxylate (3.1 g, 15 mmol), XPhos (650 mg, 1.4 mmol), XPhos Pd G3 (1.2 g, 1.4 mmol) and Cs₂CO₃ (13.3 g, 40.8 mmo) in THF (50 mL) was stirred for 2 h at 100° C. under nitrogen atmosphere. The resulting mixture was concentrated under reduced pressure. The residue was purified by silica gel column chromatography, eluted with PE/EA (1:1) to afford tert-butyl (3R,4S)-3-((2-(1,3-dioxolan-2-yl)-3-(2,2,2-trifluoroethyl)benzo[b]thiophen-7-yl)amino)-4-fluoropyrrolidine-1-carboxylate (4.1 g) as a solid. LCMS [M+H]⁺ m/z: 491.2.

### Step 7. Tert-butyl (3S,4R)-3-fluoro-4-((2-formyl-3-(2,2,2-trifluoroethyl)benzo[b]thiophen-7-yl)amino)pyrrolidine-1-carboxylate

A solution of tert-butyl (3R,4S)-3-((2-(1,3-dioxolan-2-yl)-3-(2,2,2-trifluoroethyl)benzo[b]thiophen-7-yl)amino)-4-fluoropyrrolidine-1-carboxylate (3.6 g, 7.3 mmol) in THF (15 mL), H₂O (15 mL) and AcOH (15 mL) was stirred for 2 h at 60° C. The resulting mixture was concentrated under reduced pressure. The residue was purified by silica gel column chromatography, eluted with DCM/MeOH (10:1) to afford tert-butyl (3S,4R)-3-fluoro-4-((2-formyl-3-(2,2,2-trifluoroethyl)benzo[b]thiophen-7-yl)amino)pyrrolidine-1-carboxylate (3 g) as a solid. LCMS [M+H]⁺ m/z: 447.2.

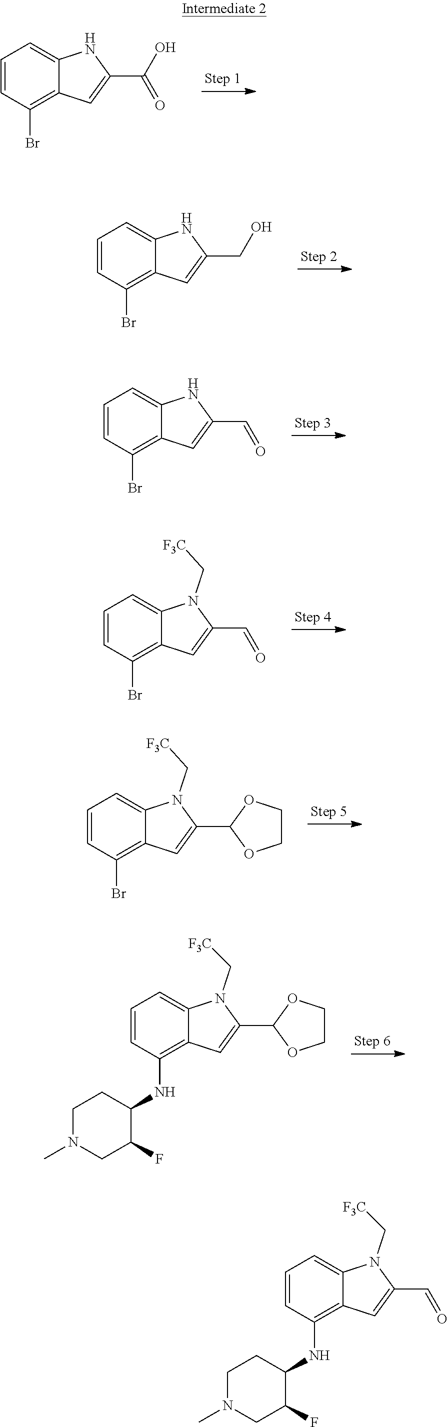

### Step 1. (4-bromo-1H-indol-2-yl)methanol

To a stirred solution of 4-bromo-1H-indole-2-carboxylic acid (30 g, 125 mmol) in THF (400 mL) was added LiAlH₄ (2.5 M in THF, 100 mL, 250 mmol) at 0° C. under nitrogen atmosphere. The resulting mixture was stirred for 2 h at rt under nitrogen atmosphere. The reaction was quenched by the addition of sat. NH₄Cl (aq.) at 0° C. The resulting mixture was extracted with EA. The combined organic layers were dried over anhydrous Na₂SO₄. After filtration, the filtrate was concentrated under reduced pressure. The residue was purified by silica gel column chromatography, eluted with DCM/MeOH (10:1) to afford (4-bromo-1H-indol-2-yl)methanol (22 g) as a solid. LCMS [M+H]⁺ m/z: 226.2.

### Step 2. 4-bromo-1H-indole-2-carbaldehyde

A mixture of (4-bromo-1H-indol-2-yl)methanol (20 g, 88 mmol) and MnO₂ (154 g, 1.8 mol) in DCM (400 mL) was stirred for 8 h at 40° C. under nitrogen atmosphere. The resulting mixture was filtered, the filter cake was washed with DCM. The filtrate was concentrated under reduced pressure. The residue was purified by silica gel column chromatography, eluted with PE/EA (5:1) to afford 4-bromo-1H-indole-2-carbaldehyde (6.5 g) as a solid. LCMS [M+H]⁺ m/z: 223.9.

### Step 3. 4-bromo-1-(2,2,2-trifluoroethyl)-1H-indole-2-carbaldehyde

To a stirred mixture of 4-bromo-1H-indole-2-carbaldehyde (5.5 g, 25 mmol) in DMF (50 mL) was added NaH (60 wt %, 2.0 g, 50 mmol) in portions at 0° C. under nitrogen atmosphere. The resulting mixture was stirred for 30 min at 0° C. under nitrogen atmosphere. To the above mixture was added 2,2,2-trifluoroethyl trifluoromethanesulfonate (8.6 g, 37 mmol) dropwise at 0° C. The resulting mixture was stirred for additional 1 h at rt. The reaction was quenched with water/ice at 0° C. The resulting mixture was extracted with EA (3×500 mL). The combined organic layers were dried over anhydrous Na₂SO₄. After filtration, the filtrate was concentrated under reduced pressure. The residue was purified by silica gel column chromatography, eluted with PE/EA (3:1) to afford 4-bromo-1-(2,2,2-trifluoroethyl)-1H-indole-2-carbaldehyde (5.2 g) as a solid. LCMS [M+H]⁺ m/z: 305.9.

### Step 4. 4-bromo-2-(1,3-dioxolan-2-yl)-1-(2,2,2-trifluoroethyl)-1H-indole

To a stirred mixture of 4-bromo-1-(2,2,2-trifluoroethyl)-1H-indole-2-carbaldehyde (2.3 g, 7.5 mmol) and ethane-1,2-diol (4.7 g, 75 mmol) in toluene (23 mL) was added TsOH·H₂O (1.7 g, 9 mmol) at rt under nitrogen atmosphere. The resulting mixture was stirred for 16 h at 120° C. under nitrogen atmosphere. The mixture was poured into sat. NaHCO₃ (aq.). The resulting mixture was extracted with EA (3×80 mL). The combined organic layers were dried over anhydrous Na₂SO₄. After filtration, the filtrate was concentrated under reduced pressure. The residue was purified by silica gel column chromatography, eluted with PE/EA (5:1) to afford 4-bromo-2-(1,3-dioxolan-2-yl)-1-(2,2,2-trifluoroethyl)-1H-indole (1.5 g) as a solid. LCMS [M+H]⁺ m/z: 350.0.

### Step 5. 2-(1,3-dioxolan-2-yl)-N-((3S,4R)-3-fluoro-1-methylpiperidin-4-yl)-1-(2,2,2-trifluoroethyl)-1H-indol-4-amine

To a stirred mixture of 4-bromo-2-(1,3-dioxolan-2-yl)-1-(2,2,2-trifluoroethyl)-1H-indole (1 g, 2.9 mmol) and (3S,4R)-3-fluoro-1-methylpiperidin-4-amine dihydrochloride (644 mg, 3.1 mmol) in toluene (10 mL) were added XPhos (136 mg, 0.3 mmol), XPhos Pd G3 (242 mg, 0.3 mmol) and Cs₂CO₃ (5.6 g, 17 mmol) at rt under nitrogen atmosphere. The resulting mixture was stirred for 2 h at 100° C. under nitrogen atmosphere. The resulting mixture was concentrated under reduced pressure. The residue was purified by silica gel column chromatography, eluted with DCM/MeOH (10:1) to afford 2-(1,3-dioxolan-2-yl)-N-((3S,4R)-3-fluoro-1-methylpiperidin-4-yl)-1-(2,2,2-trifluoroethyl)-1H-indol-4-amine (1 g) as a solid. LCMS [M+H]⁺ m/z: 402.2.

### Step 6. 4-(((3S,4R)-3-fluoro-1-methylpiperidin-4-yl)amino)-1-(2,2,2-trifluoroethyl)-1H-indole-2-carbaldehyde

A solution of 2-(1,3-dioxolan-2-yl)-N-((3S,4R)-3-fluoro-1-methylpiperidin-4-yl)-1-(2,2,2-trifluoroethyl)-1H-indol-4-amine (400 mg, 1 mmol) in HCl (5 M, aq., 15 mL) was stirred for 3 h at rt under nitrogen atmosphere. The mixture was basified to pH 8 with sat. Na₂CO₃ (aq.). The resulting mixture was extracted with EA (3×50 mL). The combined organic layers were dried over anhydrous Na₂SO₄. After filtration, the filtrate was concentrated under reduced pressure. The residue was purified by silica gel column chromatography, eluted with DCM/MeOH (10:1) to afford 4-(((3S,4R)-3-fluoro-1-methylpiperidin-4-yl)amino)-1-(2,2,2-trifluoroethyl)-1H-indole-2-carbaldehyde (210 mg) as a solid. LCMS [M+H]⁺ m/z: 358.0.

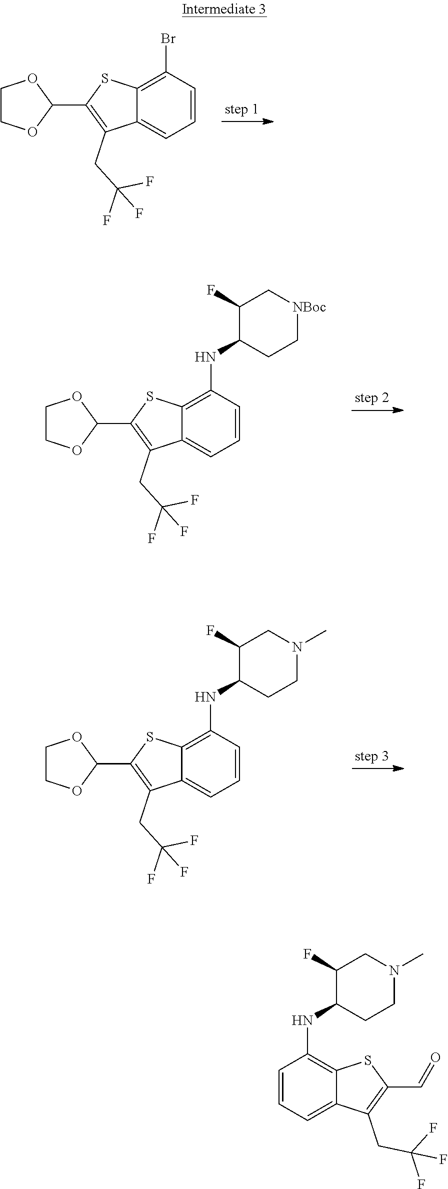 [SMILES: FC(F)(F)Cc1c(C2OCCO2)sc2c(Br)cccc12 ; CC(C)(C)OC(=O)N1CC[C@@H](Nc2cccc3c(CC(F)(F)F)c(C4OCCO4)sc23)[C@@H](F)C1 ; CN1CC[C@@H](Nc2cccc3c(CC(F)(F)F)c(C4OCCO4)sc23)[C@@H](F)C1 ; CN1CC[C@@H](Nc2cccc3c(CC(F)(F)F)c(C=O)sc23)[C@@H](F)C1]

### Step 1. Tert-butyl (3S,4R)-4-((2-(1,3-dioxolan-2-yl)-3-(2,2,2-trifluoroethyl)benzo[b]thiophen-7-yl)amino)-3-fluoropiperidine-1-carboxylate

A mixture of 2-(7-bromo-3-(2,2,2-trifluoroethyl)benzo[b]thiophen-2-yl)-1,3-dioxolane (2 g, 5.4 mmol), tert-butyl (3S,4R)-4-amino-3-fluoropiperidine-1-carboxylate (1.3 g, 6 mmol), XPhos (259.7 mg, 0.5 mmol), XPhos Pd G3 (461 mg, 0.5 mmol) and Cs₂CO₃ (5.3 g, 16.3 mmol) in THF (30 mL) was stirred for 4 h at 100° C. The resulting mixture was concentrated under reduced pressure. The residue was added H₂O (150 mL) and then extracted with EA (3×150 mL). The combined organic layers were washed with brine (3×100 mL), dried over anhydrous Na₂SO₄. After filtration, the filtrate was concentrated under reduced pressure. The residue was purified by silica gel column chromatography, eluted with EA in PE (30%-60%) to afford tert-butyl (3S,4R)-4-((2-(1,3-dioxolan-2-yl)-3-(2,2,2-trifluoroethyl)benzo[b]thiophen-7-yl)amino)-3-fluoropiperidine-1-carboxylate (1.3 g) as a solid. LCMS [M⁺1]⁺ m/z: 505.1.

### Step 2. (3S,4R)—N-(2-(1,3-dioxolan-2-yl)-3-(2,2,2-trifluoroethyl)benzo[b]thiophen-7-yl)-3-fluoro-1-methylpiperidin-4-amine

To a stirred solution of tert-butyl (3S,4R)-4-((2-(1,3-dioxolan-2-yl)-3-(2,2,2-trifluoroethyl)benzo[b]thiophen-7-yl)amino)-3-fluoropiperidine-1-carboxylate (370 mg, 0.7 mmol) in THF (5 mL) was added LiAlH₄ (2.5 M in THF, 1.8 mL, 4.4 mmol) dropwise at rt under nitrogen atmosphere. The resulting mixture was stirred for an additional 4 h at 50° C. The reaction was quenched by the addition of 10% NaOH (aq., 2 mL) at 0° C. The resulting mixture was filtered, and the filter cake was washed with DCM (3×10 mL). The filtrate was concentrated under reduced pressure. The residue was purified by silica gel column chromatography, eluted with DCM/MeOH (10:1) to afford (3S,4R)—N-(2-(1,3-dioxolan-2-yl)-3-(2,2,2-trifluoroethyl)benzo[b]thiophen-7-yl)-3-fluoro-1-methylpiperidin-4-amine (180 mg) as a solid. LCMS [M+H]⁺ m/z: 419.1.

### Step 3. 7-(((3S,4R)-3-fluoro-1-methylpiperidin-4-yl)amino)-3-(2,2,2-trifluoroethyl)benzo[b]thiophene-2-carbaldehyde

To a stirred solution of (3S,4R)—N-(2-(1,3-dioxolan-2-yl)-3-(2,2,2-trifluoroethyl)benzo[b]thiophen-7-yl)-3-fluoro-1-methylpiperidin-4-amine (180 mg, 0.4 mmol) in THF (0.8 mL) was added H₂O (0.8 mL) and AcOH (0.8 mL) dropwise at rt. The resulting mixture was stirred for additional 4 h at 60° C. The reaction mixture was concentrated under reduced pressure. The residue was purified by silica gel column chromatography, eluted with DCM/MeOH (10:1) to afford 7-(((3S,4R)-3-fluoro-1-methylpiperidin-4-yl)amino)-3-(2,2,2-trifluoroethyl)benzo[b]thiophene-2-carbaldehyde (120 mg) as a solid. LCMS [M+H]⁺ m/z: 375.0.

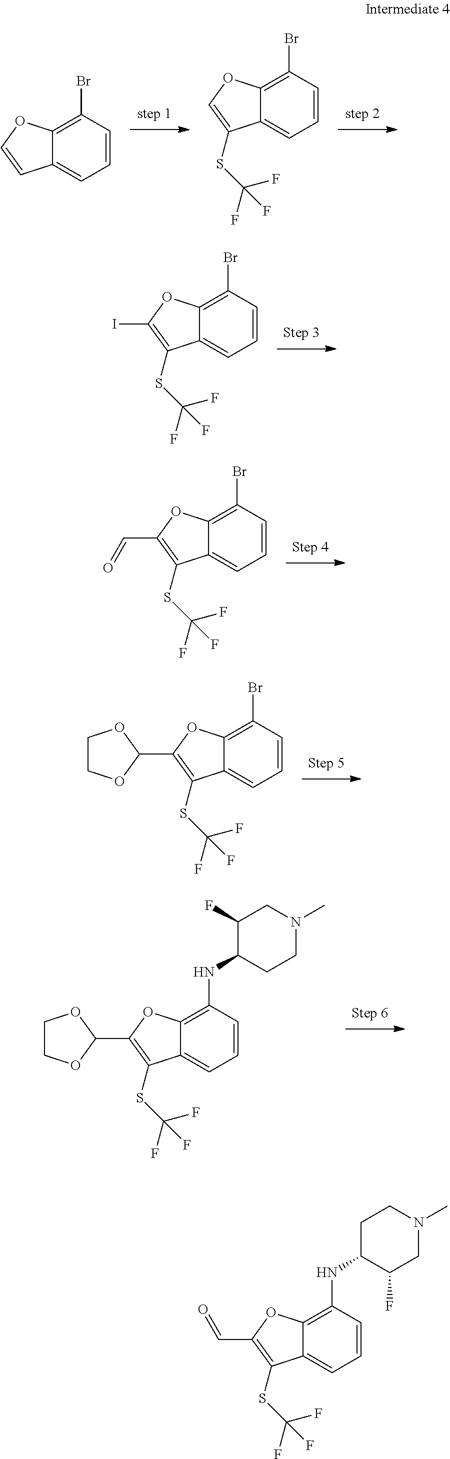

### Step 1. 7-bromo-3-((trifluoromethyl)thio)benzofuran

A solution of 7-bromobenzofuran (3 g, 15.2 mmol) and N-(phenylsulfonyl)-N-((trifluoromethyl)thio)benzenesulfonamide (12.1 g, 30.5 mmol) in DMF (60 mL) was stirred for 1 h at 80° C. under nitrogen atmosphere. To the above reaction mixture was added H₂O (200 mL) and Et₂O (1 L) and the organic phase was separated. The organic solution was washed with H₂O (3×200 mL), dried over Na₂SO₄ and then concentrated under reduce pressure. The residue was purified by silica gel column chromatography, eluted with EA in PE (0%-10%) to afford 7-bromo-3-((trifluoromethyl)thio)benzofuran (2.9 g) as an oil. GCMS [M]⁺ m/z: 295.9.

### Step 2. 7-bromo-2-iodo-3-((trifluoromethyl)thio)benzofuran

To a stirred solution of 7-bromo-3-((trifluoromethyl)thio)benzofuran (1.5 g, 5 mmol) and I₂ (1.4 g, 5.5 mmol) in THF (20 mL) was added LiHMDS (1.3 M in THF, 8.2 mL, 10.6 mmol) dropwise at −40° C. under nitrogen atmosphere. The resulting mixture was stirred for 30 min at −40° C. under nitrogen atmosphere. Then I₂ (1.4 g, 5.5 mmol) in THF (4 mL) was added. The resulting mixture was stirred for additional 1 h at −40° C. under nitrogen atmosphere. The reaction was quenched with sat. Na₂S₂O₃ (aq., 20 mL) and sat. NH₄Cl (aq., 10 mL) at 0° C. The resulting mixture was extracted with EA (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with EA in PE (0%-10%) to afford 7-bromo-2-iodo-3-((trifluoromethyl)thio)benzofuran (2.1 g) as a solid. GCMS [M]⁺ m/z: 421.8.

### Step 3. 7-bromo-3-((trifluoromethyl)thio)benzofuran-2-carbaldehyde

To a stirred solution of 7-bromo-2-iodo-3-((trifluoromethyl)thio)benzofuran (2 g, 4.7 mmol) in THF (30 mL) was added iPrMgCl·LiCl (1.3 M in THF, 4.4 mL, 5.7 mmol) dropwise at −78° C. under nitrogen atmosphere. The resulting mixture was stirred at −78° C. for 30 min under nitrogen atmosphere. Then a solution of DMF (0.4 mL, 5.7 mmol) in THF (10 mL) was added into the above reaction mixture and the resulting mixture was stirred at rt for additional 1 h under nitrogen atmosphere. The reaction was quenched with sat. NH₄Cl (aq., 30 mL) at 0° C. The resulting mixture was extracted with EA (3×100 mL). The combined organic layers were washed with water (3×50 mL), dried over anhydrous Na₂SO₄. After filtration, the filtrate was concentrated under reduced pressure. The residue was purified by silica gel column chromatography, eluted with EA in PE (0%-20%) to afford 7-bromo-3-((trifluoromethyl)thio)benzofuran-2-carbaldehyde (1.3 g) as an oil. GCMS [M]⁺ m/z: 323.9.

### Step 4. 7-bromo-2-(1,3-dioxolan-2-yl)-3-((trifluoromethyl)thio)benzofuran

To a stirred solution of 7-bromo-3-((trifluoromethyl)thio)benzofuran-2-carbaldehyde (1.2 g, 3.7 mmol) and TsOH (31.8 mg, 0.2 mmol) in toluene (1 mL) was added ethane-1,2-diol (0.4 mL, 7.4 mmol) dropwise at rt under nitrogen atmosphere. The resulting mixture was refluxed at 140° C. for 16 h under nitrogen atmosphere. Then to the above reaction mixture was diluted with water (40 mL). The resulting mixture was extracted with EA (3×100 mL). The combined organic layers were washed with NaHCO₃ (aq., 3×30 mL), dried over anhydrous Na₂SO₄. After filtration, the filtrate was concentrated under reduced pressure to afford 7-bromo-2-(1,3-dioxolan-2-yl)-3-[(trifluoromethyl) sulfanyl]-1-benzofuran (1 g, crude) as an oil. The crude product was used in the next step directly without further purification. GCMS [M]⁺ m/z: 368.0.

### Step 5. (3S,4R)—N-(2-(1,3-dioxolan-2-yl)-3-((trifluoromethyl)thio)benzofuran-7-yl)-3-fluoro-1-methylpiperidin-4-aminepiperidin-4-amine

To a stirred solution of 7-bromo-2-(1,3-dioxolan-2-yl)-3-[(trifluoromethyl) sulfanyl]-1-benzofuran (950 mg, 2.6 mmol) and (3S,4R)-3-fluoro-1-methylpiperidin-4-amine hydrochloride (634 mg, 3.1 mmol) in 1,4-dioxane (10 mL) were added Pd-PEPPSI-IHeptCl (CAS: 1814936-54-3) (250 mg, 0.3 mmol) and Cs₂CO₃ (5 g, 15.4 mmol) at rt under nitrogen atmosphere. The resulting mixture was stirred at 90° C. for 16 h under nitrogen atmosphere. The reaction mixture was concentrated under reduced pressure. The residue was purified by silica gel column chromatography, eluted with MeOH in DCM (0%-10%) to afford (3S,4R)—N-(2-(1,3-dioxolan-2-yl)-3-((trifluoromethyl)thio)benzofuran-7-yl)-3-fluoro-1-methylpiperidin-4-aminepiperidin-4-amine (630 mg) as an oil. LCMS [M+H]⁺ m/z: 421.0.

### Step 6. 7-(((3S,4R)-3-fluoro-1-methylpiperidin-4-yl)amino)-3-((trifluoromethyl)thio)benzofuran-2-carbaldehyde

To a stirred solution of (3S,4R)—N-(2-(1,3-dioxolan-2-yl)-3-((trifluoromethyl)thio)benzofuran-7-yl)-3-fluoro-1-methylpiperidin-4-amine (300 mg, 0.7 mmol) in THF (3 mL) were added AcOH (3 mL) and H₂O (3 mL) at rt. The resulting mixture was stirred at 100° C. for 16 h. The reaction mixture was concentrated under reduced pressure. The residue was purified by reversed-phase flash chromatography with the following conditions: column, C18 silica gel; mobile phase, ACN in water (10 mmol/L NH₄HCO₃), 10% to 65% gradient in 30 min; detector, UV 254 nm. This resulted in 7-(((3S,4R)-3-fluoro-1-methylpiperidin-4-yl)amino)-3-((trifluoromethyl)thio)benzofuran-2-carbaldehyde (115 mg) as a solid. LCMS [M+H]⁺ m/z: 377.1.

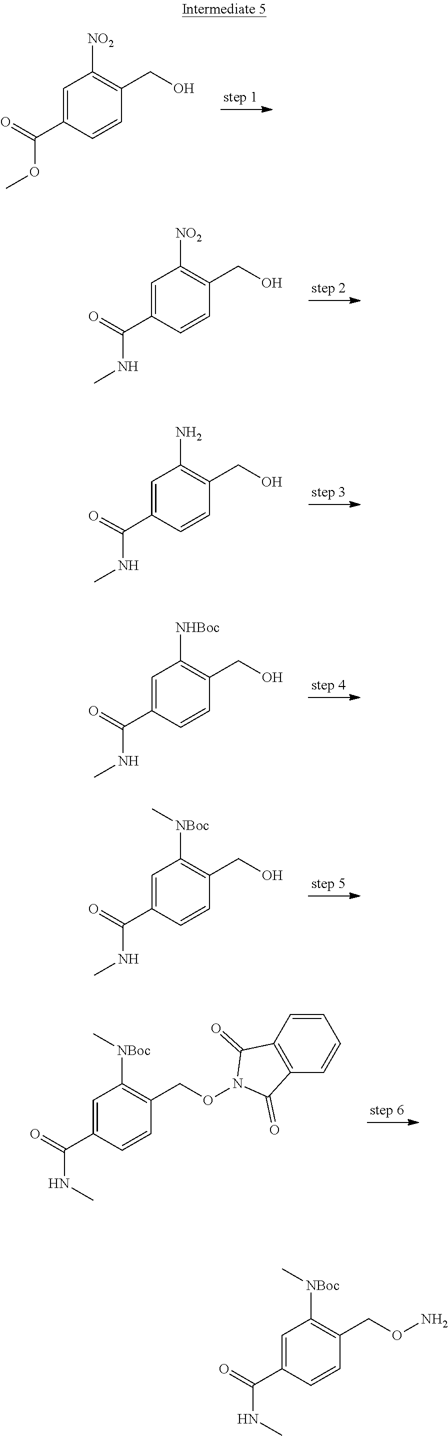

### Step 1. 4-(hydroxymethyl)-N-methyl-3-nitrobenzamide

A solution of methyl 4-(hydroxymethyl)-3-nitrobenzoate (900 mg, 4.2 mmol) and methylamine (30 wt % in MeOH, 10 mL) was stirred at 60° C. for 16 h. The resulting mixture was concentrated under reduced pressure. This resulted in crude 4-(hydroxymethyl)-N-methyl-3-nitrobenzamide (750 mg) as a solid, which was used in the next step directly without further purification. LCMS [M⁺1]⁺ m/z: 211.2.

### Step 2. 3-amino-4-(hydroxymethyl)-N-methylbenzamide

A mixture of 4-(hydroxymethyl)-N-methyl-3-nitrobenzamide (600 mg, 2.8 mmol) and Pd/C (10 wt %, 200 mg, 0.2 mmol) in MeOH (20 mL) was stirred at rt for 2 h under hydrogen atmosphere. The resulting mixture was filtered, the filter cake was washed with MeOH (3×5 mL). The filtrate was concentrated under reduced pressure. The residue was purified by trituration with DCM/MeOH (10:1). This resulted in 3-amino-4-(hydroxymethyl)-N-methylbenzamide (420 mg) as a solid. LCMS [M⁺1]⁺ m/z: 181.1.

### Step 3. Tert-butyl (2-(hydroxymethyl)-5-(methylcarbamoyl)phenyl)carbamate

A solution of 3-amino-4-(hydroxymethyl)-N-methylbenzamide (150 mg, 0.8 mmol) and Boc₂O (181.6 mg, 0.8 mmol) in EtOH (10 mL) was stirred at 50° C. for 8 h. To the above mixture was added additional Boc₂O (181.6 mg, 0.8 mmol) at rt. The resulting mixture was stirred at 50° C. for additional 8 h. The resulting mixture was concentrated under reduced pressure. The residue was purified by silica gel column chromatography, eluted with MeOH in DCM (0%-20%) to afford tert-butyl (2-(hydroxymethyl)-5-(methylcarbamoyl)phenyl)carbamate (220 mg) as a solid. LCMS [M⁺1]⁺ m/z: 281.0.

### Step 4. Tert-butyl (2-(hydroxymethyl)-5-(methylcarbamoyl)phenyl)(methyl)carbamate

To a stirred solution of tert-butyl (2-(hydroxymethyl)-5-(methylcarbamoyl)phenyl)carbamate (220 mg, 0.8 mmol) and MeI (133.6 mg, 0.9 mmol) in DMF (8 mL) was added Cs₂CO₃ (383.5 mg, 1.1 mmol) in portions at rt. The resulting mixture was stirred at rt for 2 h. The reaction mixture was diluted with water (60 mL), then extracted with EA (3×80 mL). The combined organic layers were washed with brine (300 mL), dried over anhydrous Na₂SO₄. After filtration, the filtrate was concentrated under reduced pressure. The residue was purified by silica gel column chromatography, eluted with MeOH in DCM (0%-10%) to afford tert-butyl (2-(hydroxymethyl)-5-(methylcarbamoyl)phenyl)(methyl)carbamate (120 mg) as a solid. LCMS [M⁺1]⁺ m/z: 295.0.

### Step 5. Tert-butyl (2-(((1,3-dioxoisoindolin-2-yl)oxy)methyl)-5-(methylcarbamoyl)phenyl)(methyl)carbamate

To a stirred solution of tert-butyl (2-(hydroxymethyl)-5-(methylcarbamoyl)phenyl)(methyl)carbamate (136 mg, 0.4 mmol), NHPI (75.3 mg, 0.4 mmol) and PPh₃ (121.1 mg, 0.4 mmol) in THF (4 mL) was added the solution of DIAD (93.4 mg, 0.4 mmol) in THF (2 mL) dropwise at rt under nitrogen atmosphere. The resulting mixture was stirred at rt for 2 h under nitrogen atmosphere. The reaction mixture was concentrated under reduced pressure. The residue was purified by silica gel column chromatography, eluted with MeOH in DCM (0%-10%) to afford tert-butyl (2-(((1,3-dioxoisoindolin-2-yl)oxy)methyl)-5-(methylcarbamoyl)phenyl)(methyl)carbamate (300 mg, crude) as a solid, which was used in the next step directly without further purification. LCMS [M⁺1]⁺ m/z: 440.1.

### Step 6. Tert-butyl (2-((aminooxy)methyl)-5-(methylcarbamoyl)phenyl)(methyl)carbamate

A mixture of tert-butyl (2-(((1,3-dioxoisoindolin-2-yl)oxy)methyl)-5-(methylcarbamoyl)phenyl)(methyl)carbamate (300 mg, 0.6 mmol) and MeNH₂ (30 wt % in EtOH, 10 mL) was stirred at 50° C. for 1 h. The resulting mixture was concentrated under reduced pressure. The residue was purified by reversed-phase flash chromatography with the following conditions: column, C18 silica gel; mobile phase, ACN in water (10 mmol/L NH₄HCO₃), 20% to 40% gradient in 20 min; detector, UV 254 nm. This resulted in tert-butyl (2-((aminooxy)methyl)-5-(methylcarbamoyl)phenyl)(methyl)carbamate (88 mg) as a solid. LCMS [M⁺1]⁺ m/z: 310.2.

### Example 1-1

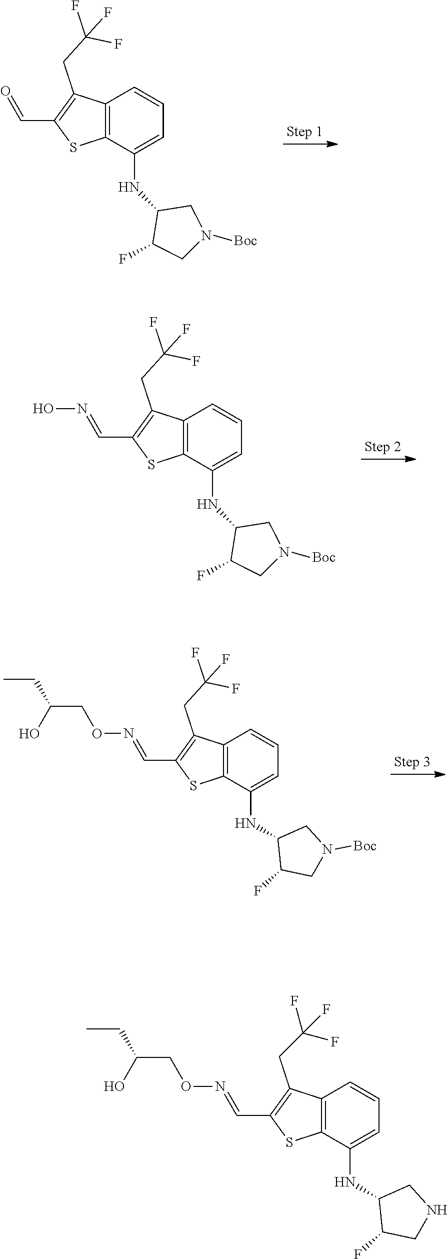 [SMILES: CC(C)(C)OC(=O)N1C[C@H](F)[C@H](Nc2cccc3c(CC(F)(F)F)c(C=O)sc23)C1 ; CC(C)(C)OC(=O)N1C[C@H](F)[C@H](Nc2cccc3c(CC(F)(F)F)c(/C=N/O)sc23)C1 ; CC[C@@H](O)CO/N=C/c1sc2c(N[C@@H]3CN(C(=O)OC(C)(C)C)C[C@@H]3F)cccc2c1CC(F)(F)F ; CC[C@@H](O)CO/N=C/c1sc2c(N[C@@H]3CNC[C@@H]3F)cccc2c1CC(F)(F)F]

### Step 1. Tert-butyl (3S,4R)-3-fluoro-4-((2-((E)-(hydroxyimino)methyl)-3-(2,2,2-trifluoroethyl)benzo[b]thiophen-7-yl)amino)pyrrolidine-1-carboxylate

To a stirred mixture of NH₂OH·HCl (0.4 g, 5.4 mmol) in EtOH (100 mL) was added TEA (0.8 g, 8.1 mmol) dropwise at rt. The resulting mixture was stirred for 10 min at rt. To the above mixture was added tert-butyl (3S,4R)-3-fluoro-4-((2-formyl-3-(2,2,2-trifluoroethyl)benzo[b]thiophen-7-yl)amino)pyrrolidine-1-carboxylate (1.2 g, 2.7 mmol) and AcOH (1.0 g, 16.2 mmol) at rt. The resulting mixture was stirred for additional 1 h at rt. The resulting mixture was concentrated under reduced pressure. The residue was purified by silica gel column chromatography, eluted with DCM/MeOH (9:1) to afford tert-butyl (3S,4R)-3-fluoro-4-((2-((E)-(hydroxyimino)methyl)-3-(2,2,2-trifluoroethyl)benzo[b]thiophen-7-yl)amino)pyrrolidine-1-carboxylate (1.1 g) as a solid. LCMS [M+H]⁺ m/z: 461.9.

### Step 2. Tert-butyl (3S,4R)-3-fluoro-4-((2-((E)-(((R)-2-hydroxybutoxy)imino)methyl)-3-(2,2,2-trifluoroethyl)benzo[b]thiophen-7-yl)amino)pyrrolidine-1-carboxylate

To a stirred mixture of tert-butyl (3S,4R)-3-fluoro-4-((2-((E)-(hydroxyimino)methyl)-3-(2,2,2-trifluoroethyl)benzo[b]thiophen-7-yl)amino)pyrrolidine-1-carboxylate (50 mg, 0.1 mmol) and LiBr (28.3 mg, 0.3 mmol) in ACN (2 mL) were added K₂CO₃ (45 mg, 0.3 mmol) and (R)-2-ethyloxirane (47 mg, 0.7 mmol) at rt. The resulting mixture was stirred for 16 h at 60° C. The reaction mixture was diluted with water (20 mL). The resulting mixture was extracted with EA (3×20 mL). The combined organic layers were dried over anhydrous Na₂SO₄. After filtration, the filtrate was concentrated under reduced pressure. This resulted in tert-butyl (3S,4R)-3-fluoro-4-((2-((E)-(((R)-2-hydroxybutoxy)imino)methyl)-3-(2,2,2-trifluoroethyl)benzo[b]thiophen-7-yl)amino)pyrrolidine-1-carboxylate (40 mg) as a solid. LCMS [M+H]⁺ m/z: 534.3.

### Step 3. (E)-7-(((3R,4S)-4-fluoropyrrolidin-3-yl)amino)-3-(2,2,2-trifluoroethyl)benzo[b]thiophene-2-carbaldehyde O—((R)-2-hydroxybutyl)oxime

To a stirred solution of tert-butyl (3S,4R)-3-fluoro-4-((2-((E)-(((R)-2-hydroxybutoxy)imino)methyl)-3-(2,2,2-trifluoroethyl)benzo[b]thiophen-7-yl)amino)pyrrolidine-1-carboxylate (37 mg, 0.07 mmol) in DCM (1.6 mL) was added TFA (0.4 mL) at 0° C. The resulting mixture was stirred for 2 h at rt. The resulting mixture was concentrated under reduced pressure. The crude product was purified by Prep-HPLC with the following conditions: Column: Xselect CSH C18 OBD Column 30*150 mm 5 μm; Mobile Phase A: Water (0.1% FA), Mobile Phase B: ACN; Flow rate: 60 mL/min; Gradient: 8% B to 28% B in 8 min; Wave Length: 254 nm/220 nm nm; RT1 (min): 9.2. This resulted in (E)-7-(((3R,4S)-4-fluoropyrrolidin-3-yl)amino)-3-(2,2,2-trifluoroethyl)benzo[b]thiophene-2-carbaldehyde O—((R)-2-hydroxybutyl)oxime (2.9 mg) as a solid.

The compounds in the table below were synthesized as described in Example 1-1 from appropriate intermediates.

| Example | Chemical name | |
|---|---|---|
| | Chemical structure | NMR, MS |
| | (E)-7-(((3R,4S)-4-fluoropyrrolidin-3-yl)amino)-3-(2,2,2- | |
| | trifluoroethyl)benzo[b]thiophene-2-carbaldehyde O-((R)-2- | |
| | hydroxybutyl) oxime | |
| 1-1 | 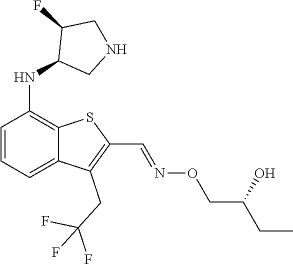 [SMILES: CC[C@@H](O)CO/N=C/c1sc2c(N[C@@H]3CNC[C@@H]3F)cccc2c1CC(F)(F)F] | 1H NMR (400 MHz, Methanol-d4) δ 8.60 (s, 1H), 8.52 (s, 1H), 7.43-7.32 (m, 2H), 6.87 (td, J = 5.3, 4.4, 2.7 Hz, 1H), 5.49-5.27 (m, 1H), 4.63-4.47 (m, 1H), 4.34-4.23 (m, 1H), 4.20- 3.80 (m, 5H), 3.78-3.38 (m, 4H), 1.76- 1.42 (m, 2H), 1.09-0.94 (m, 3H)., 19F NMR (376 MHz, MeOD) δ −66.21, −198.00, (M + H)+ 434.10. |
| | (E)-7-(((3R,4S)-4-fluoropyrrolidin-3-yl)amino)-3-(2,2,2- | |
| | trifluoroethyl)benzo[b]thiophene-2-carbaldehyde O-((S)-1- | |
| | hydroxybutan-2-yl) oxime | |
| 1-2 | 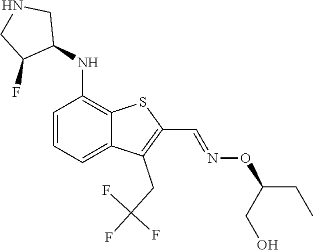 [SMILES: CC[C@@H](CO)O/N=C/c1sc2c(N[C@@H]3CNC[C@@H]3F)cccc2c1CC(F)(F)F] | 1H NMR (400 MHz, Methanol-d4) δ 8.55 (s, 1H), 7.41 (m, 2H), 6.90 (d, J = 6.9, 1.5 Hz, 1H), 5.43 (m, 1H), 4.76- 4.60 (m, 1H), 4.20-3.99 (m, 5H), 3.88- 3.64 (m, 3H), 3.50 (m, 1H), 1.68- 1.49 (m, 2H), 1.05 m, 3H), 19F NMR (377 MHz, MeOD) δ −66.02, −76.94, −198.66, (M + H)+ 434.10. |
| | (E)-7-(((3R,4S)-4-fluoropyrrolidin-3-yl)amino)-3-(2,2,2- | |
| | trifluoroethyl)benzo[b]thiophene-2-carbaldehyde O-((S)-2- | |
| | hydroxybutyl) oxime | |
| 1-3 | 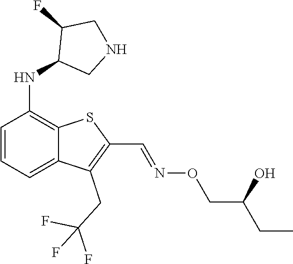 [SMILES: CC[C@H](O)CO/N=C/c1sc2c(N[C@@H]3CNC[C@@H]3F)cccc2c1CC(F)(F)F] | 1H NMR (400 MHz, Methanol-d4) δ 8.61 (s, 1H), 7.47-7.29 (m, 2H), 6.93- 6.84 (m, 1H), 5.42 ( m, 1H), 4.74-4.57 (m, 1H), 4.32-4.24 (m, 1H), 4.19- 4.01 (m, 2H), 3.97-3.62 (m, 5H), 3.48 (m, 1H), 1.75-1.42 (m, 2H), 1.08- 0.98 (m, 3H), 19F NMR (376 MHz, MeOD) δ −66.23, −76.93, −198.73, (M + H)+ 434.10. |
| | (E)-7-(((3S,4R)-4-fluoropyrrolidin-3-yl)amino)-3-(2,2,2- | |
| | trifluoroethyl)benzo[b]thiophene-2-carbaldehyde O-((R)-1- | |
| | hydroxybutan-2-yl) oxime | |
| 1-4 | 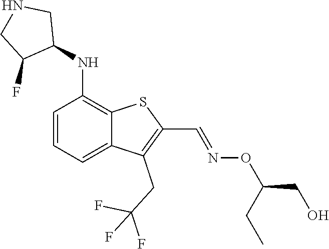 [SMILES: CC[C@H](CO)O/N=C/c1sc2c(N[C@@H]3CNC[C@@H]3F)cccc2c1CC(F)(F)F] | 1H NMR (400 MHz, Methanol-d4) δ 8.56 (s, 1H), 7.47-7.36 (m, 2H), 6.90 (d, J = 7.0, 1.4 Hz, 1H), 5.43 (d, J = 52.9, 3.1 Hz, 1H), 4.67 (m, 1H), 4.20- 4.01 (m, 5H), 3.89-3.67 (m, 3H), 3.50 (m, 1H), 1.68-1.47 (m, 2H), 1.05 (m, 3H), 19F NMR (376 MHz, MeOD) δ −66.03, −76.97, −198.69, (M + H)+ 434.10. |
| | (E)-7-(((3R,4S)-4-fluoropyrrolidin-3-yl)amino)-3-(2,2,2- | |
| | trifluoroethyl)benzo[b]thiophene-2-carbaldehyde O-(3,3,3- | |
| | trifluoro-2-hydroxypropyl) oxime | |
| 1-5 | 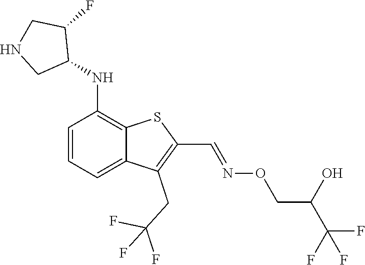 [SMILES: OC(CO/N=C/c1sc2c(N[C@@H]3CNC[C@@H]3F)cccc2c1CC(F)(F)F)C(F)(F)F] | 1H NMR (400 MHz, Methanol-d4) δ 8.65 (s, 1H), 7.40-7.30 (m, 2H), 6.90- 6.85 (m, 1H), 5.23 (dq, J = 55.4, 4.7, 3.9 Hz, 1H), 4.51-4.35 (m, 2H), 4.33- 4.17 (m, 2H), 4.01 (dq, J = 45.0, 10.6 Hz, 2H), 3.45-3.34 (m, 2H), 3.26- 3.14 (m, 1H), 2.90 (td, J = 10.2, 6.0 Hz, 1H), 19F NMR (376 MHz, MeOD) δ −66.21, −79.31, −195.74, (M + H)+ 474.10. |
| | (E)-7-(((3R,4S)-4-fluoropyrrolidin-3-yl)amino)-3-(2,2,2- | |
| | trifluoroethyl)benzo[b]thiophene-2-carbaldehyde O-(2,3- | |
| | dihydroxypropyl) oxime | |
| 1-6 | 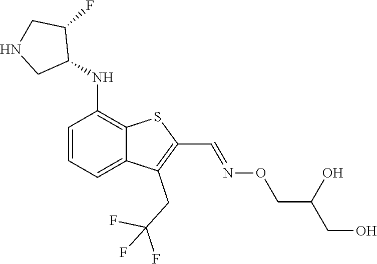 [SMILES: OCC(O)CO/N=C/c1sc2c(N[C@@H]3CNC[C@@H]3F)cccc2c1CC(F)(F)F] | 1H NMR (400 MHz, Methanol-d4) δ 8.63 (s, 1H), 8.50 (d, J = 17.1 Hz, 1H), 7.45-7.34 (m, 2H), 6.89 (p, J = 4.8 Hz, 1H), 5.39 (dd, J = 53.5, 12.1 Hz, 1H), 4.56 (dd, J = 24.2, 10.9 Hz, 1H), 4.39 (qd, J = 11.3, 5.6 Hz, 1H), 4.31-4.16 (m, 1H), 4.12-4.03 (m, 1H), 4.02- 3.90 (m, 2H), 3.79-3.57 (m, 5H), 3.43- 3.36 (m, 1H), 19F NMR (376 MHz, MeOD) δ −66.12, −66.23, −197.86, −198.03, (M + H)+ 436.10. |
| | (E)-7-(((3R,4S)-4-fluoropyrrolidin-3-yl)amino)-3-(2,2,2- | |
| | trifluoroethyl)benzo[b]thiophene-2-carbaldehyde O-(2-hydroxy- | |
| | 2-methylpropyl) oxime | |
| 1-7 | 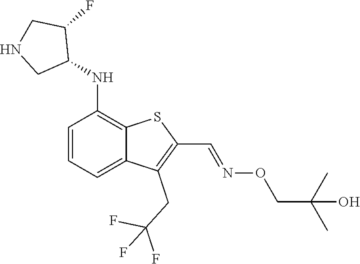 [SMILES: CC(C)(O)CO/N=C/c1sc2c(N[C@@H]3CNC[C@@H]3F)cccc2c1CC(F)(F)F] | 1H NMR (400 MHz, Methanol-d4) δ 8.66 (s, 1H), 7.48-7.32 (m, 2H), 6.96- 6.86 (m, 1H), 5.45 (ddt, J = 52.9, 12.3, 3.1 Hz, 1H), 4.66 (dddd, J = 25.4, 10.9, 7.9, 3.2 Hz, 1H), 4.25 (s, 1H), 4.08 (d, J = 7.9 Hz, 2H), 3.97 (q, J = 10.7 Hz, 1H), 3.90-3.78 (m, 2H), 3.77-3.64 (m, 1H), 3.49 (td, J = 11.1, 6.3 Hz, 1H), 1.38 (s, 2H), 1.30 (s, 4H), 19F NMR (376 MHz, MeOD) δ −66.23, −77.00, −198.71, (M + H)+ 434.10. |
| | (E)-7-(((3R,4S)-4-fluoropyrrolidin-3-yl)amino)-3-(2,2,2- | |
| | trifluoroethyl)benzo[b]thiophene-2-carbaldehyde O-(3-amino-2- | |
| | hydroxypropyl)oxime | |
| 1-8 | 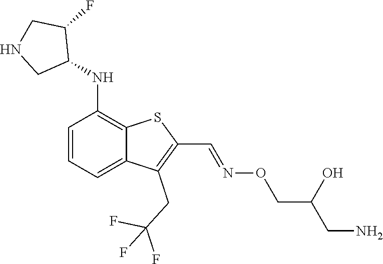 [SMILES: NCC(O)CO/N=C/c1sc2c(N[C@@H]3CNC[C@@H]3F)cccc2c1CC(F)(F)F] | 1HNMR (400 MHz, Methanol-d4) δ 8.61 (s, 1H), 7.39-7.26 (m, 2H), 6.85 (td, J = 7.4, 6.9, 1.9 Hz, 1H), 5.32-5.13 (m, 1H), 4.38-4.16 (m, 3H), 4.11- 3.87 (m, 3H), 3.79-3.36 (m, 2H), 3.29- 3.11 (m, 2H), 2.96-2.72 (m, 2H), 19F NMR (376 MHz, MeOD) δ −66.20, −195.87, (M + H)+ 435.05. |
| | (E)-7-(((3R,4S)-4-fluoropyrrolidin-3-yl)amino)-3-(2,2,2- | |
| | trifluoroethyl)benzo[b]thiophene-2-carbaldehyde O-(1-amino-3- | |
| | hydroxypropan-2-yl) oxime | |
| 1-9 | 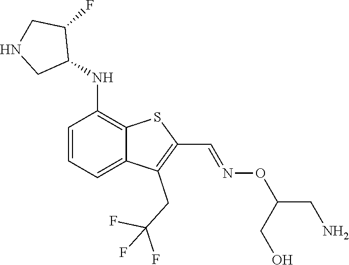 [SMILES: NCC(CO)O/N=C/c1sc2c(N[C@@H]3CNC[C@@H]3F)cccc2c1CC(F)(F)F] | 1H NMR (400 MHz, Methanol-d4) δ 8.56 (d, J = 31.3 Hz, 3H), 7.44-7.34 (m, 2H), 6.95-6.87 (m, 1H), 5.37 (d, J = 53.7 Hz, 1H), 4.54 (d, J = 25.0 Hz, 1H), 4.48-4.39 (m, 1H), 4.21 (d, J = 5.4 Hz, 2H), 4.07 (q, J = 10.6 Hz, 2H), 3.71 (dd, J = 11.4, 8.0 Hz, 1H), 3.63 (d, J = 3.5 Hz, 1H), 3.56 (s, 1H), 3.27 (s, 1H), 3.19 (dd, J = 13.0, 3.5 Hz, 1H), 3.05 (dd, J = 13.0, 8.7 Hz, 1H), 19F NMR (376 MHz, MeOD) δ −65.97, −197.66, (M + H)+ 435.10. |
| | (E)-7-(((3R,4S)-4-fluoropyrrolidin-3-yl)amino)-3-(2,2,2- | |
| | trifluoroethyl)benzo[b]thiophene-2-carbaldehyde O-(2-hydroxy- | |
| | 3-methoxypropyl) oxime | |
| 1-10 | 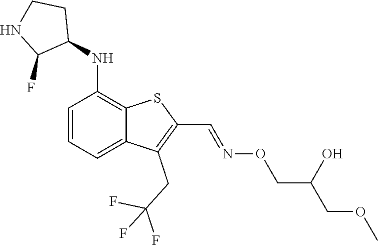 [SMILES: COCC(O)CO/N=C/c1sc2c(N[C@@H]3CCN[C@@H]3F)cccc2c1CC(F)(F)F] | 1H NMR (400 MHz, Methanol-d4) δ 8.59 (s, 1H), 7.37-7.26 (m, 2H), 6.89- 6.81 (m, 1H), 5.32-5.13 (m, 1H), 4.37- 3.89 (m, 6H), 3.64-3.34 (m, 7H), 3.25-3.15 (m, 1H), 2.88 (m, 1H), 19F NMR (376 MHz, MeOD) δ −66.19, −66.19, −76.94, −195.67, −195.68, −195.89, (M + H)+ 450.15. |
| | (E)-7-(((3R,4S)-4-fluoropyrrolidin-3-yl)amino)-3-(2,2,2- | |
| | trifluoroethyl)benzo[b]thiophene-2-carbaldehyde O-(1-hydroxy- | |
| | 3-methoxypropan-2-yl) oxime | |
| 1-11 | 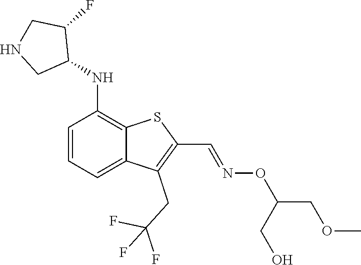 [SMILES: COCC(CO)O/N=C/c1sc2c(N[C@@H]3CNC[C@@H]3F)cccc2c1CC(F)(F)F] | 1H NMR (400 MHz, Methanol-d4) δ 8.54 (s, 1H), 7.42-7.31 (m, 2H), 6.92- 6.82 (m, 1H), 5.23 (m, 1H), 4.45-4.38 (m, 1H), 4.31-4.19 (m, 1H), 4.18- 4.12 (m, 2H), 4.04 (m, 2H), 3.55-3.46 (m, 2H), 3.44-3.32 (m, 5H), 3.26- 3.14 (m, 1H), 2.90 (d, J = 11.1, 9.3 Hz, 1H), 19F NMR (376 MHz, MeOD) δ −65.82, −65.99, −195.85, (M + H)+ 450.10. |
| | (E)-7-(((3R,4S)-4-fluoropyrrolidin-3-yl)amino)-3-(2,2,2- | |
| | trifluoroethyl)benzo[b]thiophene-2-carbaldehyde O-((3- | |
| | hydroxypyrrolidin-3-yl)methyl) oxime | |
| 1-12 | 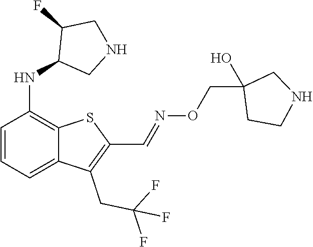 [SMILES: OC1(CO/N=C/c2sc3c(N[C@@H]4CNC[C@@H]4F)cccc3c2CC(F)(F)F)CCNC1] | 1H NMR (400 MHz, Methanol-d4) δ 8.34 (d, J = 234.6 Hz, 1H), 7.39-7.26 (m, 2H), 6.85 (t, J = 6.6 Hz, 1H), 5.21 (dq, J = 55.3, 5.1, 4.0 Hz, 1H), 4.48- 4.37 (m, 1H), 4.30 (d, J = 1.8 Hz, 1H), 4.26-4.15 (m, 1H), 4.09-3.99 (m, 1H), 3.91 (t, J = 10.7 Hz, 1H), 3.79- 3.46 (m, 1H), 3.44-3.34 (m, 1H), 3.28 -2.93 (m, 5H), 2.87 (td, J = 10.4, 6.1 Hz, 1H), 2.20-1.98 (m, 1H), 1.90 (dp, J = 13.0, 4.9 Hz, 1H), 19F NMR (376 MHz, MeOD) δ −66.17, −195.57, (M + H)+ 461.05. |
| | (E)-7-(((3R,4S)-4-fluoropyrrolidin-3-yl)amino)-3-(2,2,2- | |
| | trifluoroethyl)benzo[b]thiophene-2-carbaldehyde O-(3- | |
| | (hydroxymethyl)pyrrolidin-3-yl) oxime | |
| 1-13 | 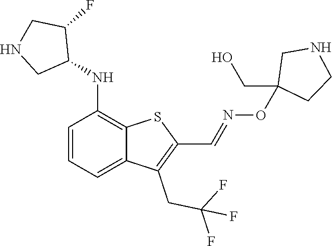 [SMILES: OCC1(O/N=C/c2sc3c(N[C@@H]4CNC[C@@H]4F)cccc3c2CC(F)(F)F)CCNC1] | 1H NMR (400 MHz, Methanol-d4) δ 8.55 (d, J = 3.0 Hz, 1H), 7.37 (d, J = 6.6 Hz, 2H), 6.92-6.83 (m, 1H), 5.23 (dt, J = 55.4, 4.1 Hz, 1H), 4.39-4.21 (m, 3H), 4.04 (q, J = 10.6 Hz, 2H), 3.79-3.47 (m, 1H), 3.40 (dd, J = 11.2, 7.6 Hz, 1H), 3.26-3.16 (m, 2H), 3.08 (q, J = 12.4 Hz, 3H), 2.90 (t, J = 10.2 Hz, 1H), 2.10- 1.88 (m, 2H), 19F NMR (376 MHz, MeOD) δ −65.98, −195.84, (M + H)+ 461.10. |
| | (E)-7-(((3R,4S)-4-fluoropyrrolidin-3-yl)amino)-3-(2,2,2- | |
| | trifluoroethyl)benzo[b]thiophene-2-carbaldehyde O-((1- | |
| | hydroxycyclopentyl)methyl) oxime | |
| 1-14 | 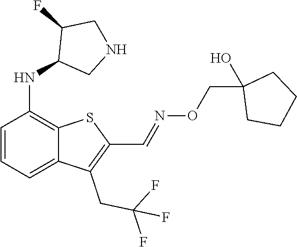 [SMILES: OC1(CO/N=C/c2sc3c(N[C@@H]4CNC[C@@H]4F)cccc3c2CC(F)(F)F)CCCC1] | 1H NMR (400 MHz, Methanol-d4) δ 8.55 (s, 1H), 7.46-7.33 (m, 2H), 6.88 (dd, J = 5.7, 2.8 Hz, 1H), 5.40 (s, 1H), 4.54-4.40 (m, 1H) 4.20 (s, 2H), 4.05 (q, J = 10.6 Hz, 2H), 3.63 (t, J = 9.8 Hz, 1H), 3.55 (s, 1H), 3.48-3.46 (m, 1H), 3.21 (d, J = 10.7 Hz, 1H), 1.96-1.63 (m, 8H), 19F NMR (377 MHz, MeOD) δ −66.04, −197.44, (M + H)+ 460.20. |
| | (E)-7-(((3R,4S)-4-fluoropyrrolidin-3-yl)amino)-3-(2,2,2- | |
| | trifluoroethyl)benzo[b]thiophene-2-carbaldehyde O-((3- | |
| | hydroxytetrahydro-2H-pyran-3-yl)methyl) oxime | |
| 1-15 | 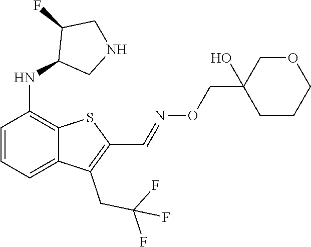 [SMILES: OC1(CO/N=C/c2sc3c(N[C@@H]4CNC[C@@H]4F)cccc3c2CC(F)(F)F)CCCOC1] | 1H NMR (400 MHz, Methanol-d4) δ 8.32 (d, J = 237.2 Hz, 1H), 7.38-7.24 (m, 2H), 6.85 (dd, J = 10.9, 5.9 Hz, 1H), 5.22 (ddt, J = 55.5, 8.0, 4.0 Hz, 1H), 4.37-4.15 (m, 3H), 3.98 (dq, J = 47.1, 10.7 Hz, 2H), 3.78-3.49 (m, 4H), 3.43- 3.35 (m, 1H), 3.28-3.12 (m, 2H), 2.88 (t, J = 10.2 Hz, 1H), 2.11-1.84 (m, 2H), 1.75-1.49 (m, 2H), 19F NMR (376 MHz, MeOD) δ −66.19, −195.88, (M + H)+ 476.10. |
| | (E)-7-(((3R,4S)-4-fluoropyrrolidin-3-yl)amino)-3-(2,2,2- | |
| | trifluoroethyl)benzo[b]thiophene-2-carbaldehyde O-(3- | |
| | (hydroxymethyl)tetrahydro-2H-pyran-3-yl) oxime | |
| 1-16 | 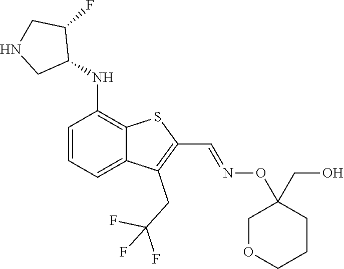 [SMILES: OCC1(O/N=C/c2sc3c(N[C@@H]4CNC[C@@H]4F)cccc3c2CC(F)(F)F)CCCOC1] | 1H NMR (400 MHz, Methanol-d4) δ 8.55 (s, 1H), 7.46-7.34 (m, 2H), 6.90 (dd, J = 6.6, 1.8 Hz, 1H), 5.46-5.30 (m, 1H), 4.58 (dd, J = 23.6, 10.6 Hz, 1H), 4.22 (s, 2H), 4.06 (q, J = 10.6 Hz, 2H), 3.79-3.57 (m, 6H), 3.50 (d, J = 11.6 Hz, 1H), 3.38 (t, J = 10.9 Hz, 1H), 1.94- 1.80 (m, 2H), 1.73 (t, J = 8.6 Hz, 2H), 19F NMR (376 MHz, MeOD) δ −66.02, −76.92, −198.10, (M + H)+ 476.15. |
| | (E)-7-(((3R,4S)-4-fluoropyrrolidin-3-yl)amino)-3-(2,2,2- | |
| | trifluoroethyl)benzo[b]thiophene-2-carbaldehyde O-(2- | |
| | hydroxycyclopentyl) oxime | |
| 1-17 | 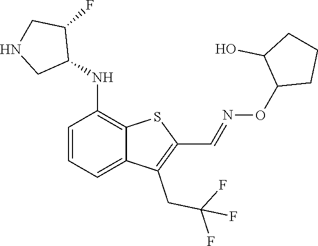 [SMILES: OC1CCCC1O/N=C/c1sc2c(N[C@@H]3CNC[C@@H]3F)cccc2c1CC(F)(F)F] | 1H NMR (400 MHz, Methanol-d4) δ 8.54 (s, 1H), 7.48-7.31 (m, 2H), 6.88 (d, J = 5.4, 3.2 Hz, 1H), 5.40 (m, 1H),4.73-4.57 (m, 1H) 4.56-4.50 (m, 1H), 4.27 (s, 1H), 3.93 (m, 2H), 3.79 (m, 3H), 3.46 m, 1H), 2.16-2.06 (m, 1H), 1.99 (m, 1H), 1.86-1.73 (m, 3H), 1.64 (m, 1H), 19F NMR (376 MHz, Methanol-d4) δ −66.12, −66.25, −76.97, −198.78, (M + H)+ 446.15. |
| | (E)-7-(((3R,4S)-4-fluoropyrrolidin-3-yl)amino)-3-(2,2,2- | |
| | trifluoroethyl)benzo[b]thiophene-2-carbaldehyde O-((1- | |
| | hydroxycyclobutyl)methyl) oxime | |
| 1-18 | 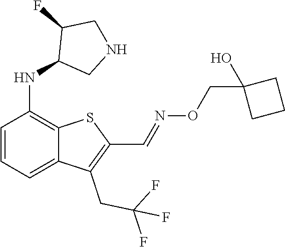 [SMILES: OC1(CO/N=C/c2sc3c(N[C@@H]4CNC[C@@H]4F)cccc3c2CC(F)(F)F)CCC1] | 1H NMR (400 MHz, Methanol-d4) δ 8.22 (d, J = 237.9 Hz, 1H), 7.29-7.16 (m, 2H), 6.79-6.71 (m, 1H), 5.23- 5.02 (m, 1H), 4.34-4.07 (m, 3H), 3.88 (dq, J = 47.5, 10.7 Hz, 2H), 3.42-3.23 (m, 2H), 3.15-3.03 (m, 1H), 2.78 (dd, J = 11.2, 9.2 Hz, 1H), 2.30-2.12 (m, 2H), 2.07-1.90 (m, 2H), 1.78-1.47 (m, 2H), 19F NMR (376 MHz, Methanol- d4) δ −66.10, −66.20, −195.82, −195.91, (M + H)+ 446.15. |
| | (E)-7-(((3R,4S)-4-fluoropyrrolidin-3-yl)amino)-3-(2,2,2- | |
| | trifluoroethyl)benzo[b]thiophene-2-carbaldehyde O-(1- | |
| | (hydroxymethyl)cyclobutyl) oxime | |
| 1-19 | 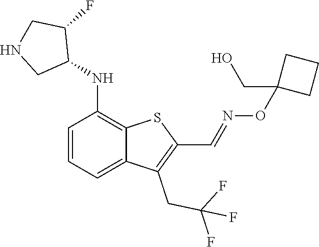 [SMILES: OCC1(O/N=C/c2sc3c(N[C@@H]4CNC[C@@H]4F)cccc3c2CC(F)(F)F)CCC1] | 1H NMR (400 MHz, Methanol-d4) δ 8.47 (s, 1H), 7.38-7.23 (m, 2H), 6.80 (dd, J = 7.1, 1.3 Hz, 1H), 5.33 (dt, J = 52.9, 3.1 Hz, 1H), 4.57 (d, J = 22.8 Hz, 1H), 4.14 (s, 2H), 4.03-3.92 (m, 2H), 3.82-3.53 (m, 3H), 3.40 (t, J = 11.1 Hz, 1H), 2.23 (tt, J = 8.8, 3.3 Hz, 2H), 2.06 (qd, J = 9.6, 2.8 Hz, 2H), 1.74 (ddt, J = 19.3, 9.7, 3.5 Hz, 1H), 1.67-1.54 (m, 1H), 19F NMR (376 MHz, Methanol- d4) δ −66.05, −76.92, −198.71, (M + H)+ 446.10. |
| | (E)-7-(((3R,4S)-4-fluoropyrrolidin-3-yl)amino)-3-(2,2,2- | |
| | trifluoroethyl)benzo[b]thiophene-2-carbaldehyde O-((4- | |
| | hydroxytetrahydro-2H-pyran-4-yl)methyl) oxime | |
| 1-20 | 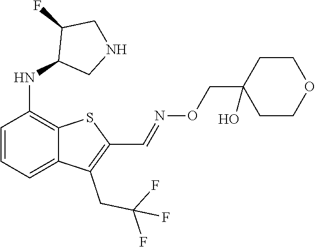 [SMILES: OC1(CO/N=C/c2sc3c(N[C@@H]4CNC[C@@H]4F)cccc3c2CC(F)(F)F)CCOCC1] | 1H NMR (400 MHz, Methanol-d4) δ 8.62 (s, 1H), 7.40-7.25 (m, 2H), 6.90- 6.80 (m, 1H), 5.21 (dq, J = 55.3, 4.5 Hz, 1H), 4.30-4.10 (m, 3H), 4.09-3.68 (m, 6H), 3.38 (dt, J = 11.1, 7.4 Hz, 2H), 3.28-3.14 (m, 1H), 2.87 (ddd, J = 11.2, 9.2, 2.4 Hz, 1H), 2.01-1.79 (m, 2H), 1.59 (ddq, J = 26.8, 14.0, 2.5 Hz, 2H), 19F NMR (376 MHz, Methanol-d4) δ −66.18, −195.87, (M + H)+ 476.10. |
| | (E)-7-(((3R,4S)-4-fluoropyrrolidin-3-yl)amino)-3-(2,2,2- | |
| | trifluoroethyl)benzo[b]thiophene-2-carbaldehyde O-(4- | |
| | (hydroxymethyl)tetrahydro-2H-pyran-4-yl) oxime | |
| 1-21 | 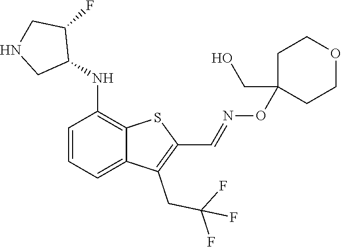 [SMILES: OCC1(O/N=C/c2sc3c(N[C@@H]4CNC[C@@H]4F)cccc3c2CC(F)(F)F)CCOCC1] | 1H NMR (400 MHz, Methanol-d4) δ 8.56 (s, 1H), 7.48-7.37 (m, 2H), 6.94- 6.88 (m, 1H), 5.43 (dt, J = 53.0, 3.1 Hz, 1H), 4.70 (s, 1H), 4.14 (s, 2H), 4.07 (q, J = 10.6 Hz, 2H), 3.87-3.61 (m, 7H), 3.50 (t, J = 11.1 Hz, 1H), 1.84 (ddd, J = 15.2, 10.4, 5.2 Hz, 2H), 1.62 (dd, J = 14.1, 2.6 Hz, 2H), 19F NMR (376 MHz, Methanol-d4) δ −66.04, −76.92, −198.72, (M + H)+ 476.15. |
| | (Z)-7-(((3R,4S)-4-fluoropyrrolidin-3-yl)amino)-3-(2,2,2- | |
| | trifluoroethyl)benzo[b]thiophene-2-carbaldehyde O-(2-hydroxy- | |
| | 2-phenylpropyl) oxime | |
| 1-22 | 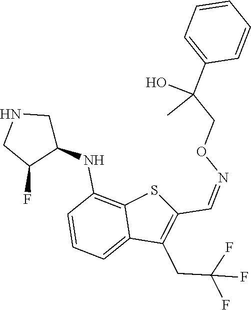 [SMILES: CC(O)(CO/N=C\c1sc2c(N[C@@H]3CNC[C@@H]3F)cccc2c1CC(F)(F)F)c1ccccc1] | 1H NMR (400 MHz, Methanol-d4) δ 8.48-7.87 (m, 1H), 7.51-7.39 (m, 2H), 7.36-7.21 (m, 4H), 7.15 (m, 1H), 6.83-6.73 (m, 1H), 5.45-5.22 (m, 1H), 4.65-4.48 (m, 1H), 4.41-4.20 (m, 2H), 3.98-3.57 (m, 5H), 3.35 (m, 1H), 1.55 (d, J = 30.7 Hz, 3H), 19F NMR (376 MHz, Methanol-d4) δ −66.19, −66.17, −66.22, −77.01, −198.53, −193.54, −198.78, (M + H)+ 496.10. |
| | (E)-7-(((3R,4S)-4-fluoropyrrolidin-3-yl)amino)-3-(2,2,2- | |
| | trifluoroethyl)benzo[b]thiophene-2-carbaldehyde O-(1-hydroxy- | |
| | 2-phenylpropan-2-yl) oxime | |
| 1-23 | 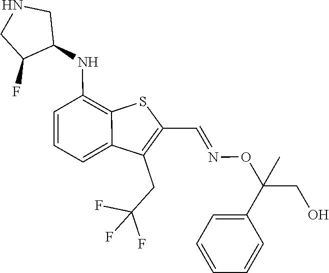 [SMILES: CC(CO)(O/N=C/c1sc2c(N[C@@H]3CNC[C@@H]3F)cccc2c1CC(F)(F)F)c1ccccc1] | 1H NMR (400 MHz, Methanol-d4) δ 8.37 (s, 1H), 7.59-7.52 (m, 2H), 7.44- 7.29 (m, 4H), 7.22 (m, 1H), 6.89 (m, 1H), 5.42 (m, 1H), 4.73-4.58 (m, 1H), 4.42 (d, J = 12.7, 5.1 Hz, 1H), 4.31 (d, J = 12.8 Hz, 1H), 3.94 (m, 2H), 3.85- 3.63 (m, 3H), 3.50 (t, J = 11.1 Hz, 1H), 1.66 (s, 3H), 19F NMR (376 MHz, Methanol-d4) δ −66.04, −66.28, −77.02, −198.74, −198.74, (M + H)+ 496.10. |
| | (E)-7-(((3R,4S)-4-fluoropyrrolidin-3-yl)amino)-3-(2,2,2- | |
| | trifluoroethyl)benzo[b]thiophene-2-carbaldehyde O-(2-hydroxy- | |
| | 2-phenylpropyl) oxime | |
| 1-24 | 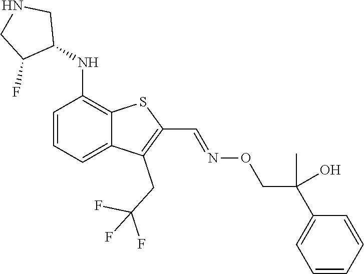 [SMILES: CC(O)(CO/N=C/c1sc2c(N[C@H]3CNC[C@H]3F)cccc2c1CC(F)(F)F)c1ccccc1] | 1H NMR (400 MHz, Methanol-d4) δ 8.57 (s, 1H), 7.56-7.51 (m, 2H), 7.38- 7.31 (m, 4H), 7.28-7.22 (m, 1H), 6.87 (m, 1H), 5.48-5.33 (m, 1H), 4.63 (m, 1H), 4.41-4.29 (m, 2H), 3.99-3.70 (m, 5H), 3.47 (m, 1H), 1.61 (s, 3H), 19F NMR (376 MHz, Methanol-d4) δ −66.13, −66.22, −76.98, −198.76, (M + H)+ 496.15. |
| | benzyl 4-(((((E)-(7-(((3R,4S)-4-fluoropyrrolidin-3-yl)amino)-3- | |
| | (2,2,2-trifluoroethyl)benzo[b]thiophen-2- | |
| | yl)methylene)amino)oxy)methyl)-4-hydroxypiperidine-1- | |
| | carboxylate | |
| 1-25 | 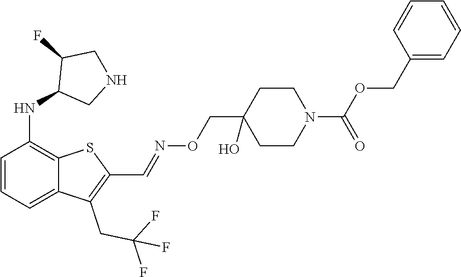 [SMILES: O=C(OCc1ccccc1)N1CCC(O)(CO/N=C/c2sc3c(N[C@@H]4CNC[C@@H]4F)cccc3c2CC(F)(F)F)CC1] | 1H NMR (400 MHz, Methanol-d4) δ 8.61 (s, 1H), 8.53 (s, 1H), 7.47-7.09 (m, 7H), 6.92-6.84 (m, 1H), 5.32 (d, J = 53.9 Hz, 1H), 5.11 (s, 2H), 4.45 (dd, J = 20.8, 10.6 Hz, 1H), 4.15 (d, J = 51.1 Hz, 2H), 4.07-3.88 (m, 4H), 3.71- 3.47 (m, 3H), 3.30-3.12 (m, 3H), 2.07- 1.49 (m, 4H), 19F NMR (376 MHz, Methanol-d4) δ −66.18, −197.52, (M + H)+ 609.20. |
| | benzyl 4-((((E)-(7-(((3R,4S)-4-fluoropyrrolidin-3-yl)amino)-3- | |
| | (2,2,2-trifluoroethyl)benzo[b]thiophen-2- | |
| | yl)methylene)amino)oxy)-4-(hydroxymethyl)piperidine-1- | |
| | carboxylate | |
| 1-26 | 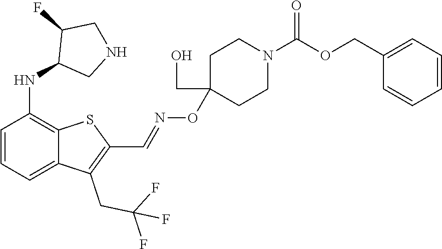 [SMILES: O=C(OCc1ccccc1)N1CCC(CO)(O/N=C/c2sc3c(N[C@@H]4CNC[C@@H]4F)cccc3c2CC(F)(F)F)CC1] | 1H NMR (400 MHz, Methanol-d4) δ 8.53 (s, 2H), 7.44-7.38 (m, 2H), 7.37- 7.25 (m, 5H), 6.89 (dd, J = 5.9, 2.4 Hz, 1H), 5.35 (d, J = 53.8 Hz, 1H), 5.12 (s, 2H), 4.52 (d, J = 24.4 Hz, 1H), 4.12 (s, 2H), 4.05 (q, J = 10.6 Hz, 2H), 3.93 (d, J = 13.5 Hz, 2H), 3.75-3.50 (m, 4H), 3.27 (d, J = 10.9 Hz, 2H), 1.69 (t, J = 4.6 Hz, 4H), 19F NMR (376 MHz, Methanol-d4) δ −66.01, −197.62, (M + H)+ 609.15. |
| | (E)-7-(((3R,4S)-4-fluoropyrrolidin-3-yl)amino)-3-(2,2,2- | |
| | trifluoroethyl)benzo[b]thiophene-2-carbaldehyde O-(2-hydroxy- | |
| | 3-(1H-pyrazol-1-yl)propyl) oxime | |
| 1-27 | 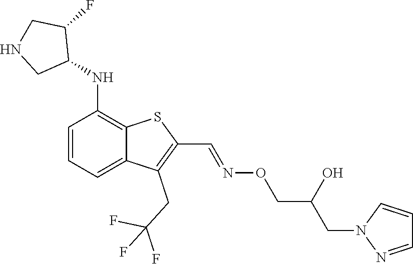 [SMILES: OC(CO/N=C/c1sc2c(N[C@@H]3CNC[C@@H]3F)cccc2c1CC(F)(F)F)Cn1cccn1] | 1H NMR (400 MHz, Methanol-d4) δ 8.37 (m, 1H), 7.68 (dd, J = 5.1, 2.3 Hz, 1H), 7.52 (t, J = 2.2 Hz, 1H), 7.45-7.32 (m, 2H), 6.93-6.85 (m, 1H), 6.31 (t, J = 2.2 Hz, 1H), 5.42 (ddt, J = 52.9, 14.9, 3.1 Hz, 1H), 4.63 (dddd, J = 25.0, 11.2, 8.5, 3.1 Hz, 1H), 4.38 (dd, J = 13.3, 3.7 Hz, 1H), 4.33-3.90 (m, 6H), 3.87- 3.61 (m, 3H), 3.55-3.43 (m, 1H), 19F NMR (376 MHz, Methanol-d4) δ −66.10, −66.20, −77.24,, −198.55, −198.56, −198.77, (M + H)+ 486.15. |
| | (E)-7-(((3R,4S)-4-fluoropyrrolidin-3-yl)amino)-3-(2,2,2- | |
| | trifluoroethyl)benzo[b]thiophene-2-carbaldehyde O-(1-hydroxy- | |
| | 3-(1H-pyrazol-1-yl)propan-2-yl) oxime | |
| 1-28 | 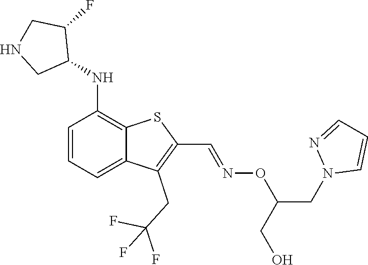 [SMILES: OCC(Cn1cccn1)O/N=C/c1sc2c(N[C@@H]3CNC[C@@H]3F)cccc2c1CC(F)(F)F] | 1H NMR (400 MHz, Methanol-d4) δ 8.58 (s, 1H), 7.71 (d, J = 2.3 Hz, 1H), 7.54 (d, J = 1.9 Hz, 1H), 7.47-7.36 (m, 2H), 6.90 (dd, J = 7.0, 1.4 Hz, 1H), 6.33 (t, J = 2.1 Hz, 1H), 5.43 (m, 1H), 4.65 (m, 2H), 4.35 (qd, J = 14.2, 5.7 Hz, 2H), 4.18-4.00 (m, 4H), 3.87-3.64 (m, 3H), 3.55-3.44 (m, 1H), 19F NMR (376 MHz, Methanol-d4) −66.01, −77.05, −198.72, (M + H)+ 486.20. |
| | (E)-7-(((3R,4S)-4-fluoropyrrolidin-3-yl)amino)-3-(2,2,2- | |
| | trifluoroethyl)benzo[b]thiophene-2-carbaldehyde O-((1- | |
| | hydroxycyclohexyl)methyl) oxime | |
| 1-29 | 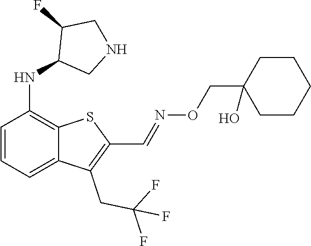 [SMILES: OC1(CO/N=C/c2sc3c(N[C@@H]4CNC[C@@H]4F)cccc3c2CC(F)(F)F)CCCCC1] | 1H NMR (400 MHz, Methanol-d4) δ 8.62 (s, 1H), 7.44-7.32 (m, 2H), 6.91- 6.84 (m, 1H), 5.41 (dt, J = 52.9, 3.1 Hz, 1H), 4.68-4.55 (m, 1H), 4.08 (s, 2H), 3.94 (q, J = 10.7 Hz, 2H), 3.86-3.63 (m, 3H), 3.47 (t, J = 11.2 Hz, 1H), 1.73- 1.31 (m, 10H), 19F NMR (376 MHz, Methanol-d4) δ −66.13, −66.23, −76.95 −198.75, −198.77, (M + H)+ 474.10. |
| | (E)-7-(((3R,4S)-4-fluoropyrrolidin-3-yl)amino)-3-(2,2,2- | |
| | trifluoroethyl)benzo[b]thiophene-2-carbaldehyde O-(1- | |
| | (hydroxymethyl)cyclohexyl) oxime | |
| 1-30 | 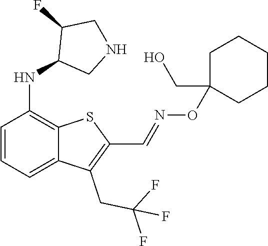 [SMILES: OCC1(O/N=C/c2sc3c(N[C@@H]4CNC[C@@H]4F)cccc3c2CC(F)(F)F)CCCCC1] | 1H NMR (400 MHz, Methanol-d4) δ 8.54 (s, 1H), 7.48-7.36 (m, 2H), 6.91 (dd, J = 7.3, 1.3 Hz, 1H), 5.42 (dt, J = 53.0, 3.1 Hz, 1H), 4.75-4.62 (m, 1H), 4.14-4.01 (m, 4H), 3.87-3.65 (m, 3H), 3.50 (t, J = 11.1 Hz, 1H), 1.77- 1.33 (m, 10H), 19F NMR (376 MHz, Methanol-d4) δ −66.07, −76.95, −198.72, (M + H)+ 474.15. |
| | (Z)-7-(((3R,4S)-4-fluoropyrrolidin-3-yl)amino)-3-(2,2,2- | |
| | trifluoroethyl)benzo[b]thiophene-2-carbaldehyde O-((1- | |
| | hydroxycyclohexyl)methyl) oxime | |
| 1-31 | 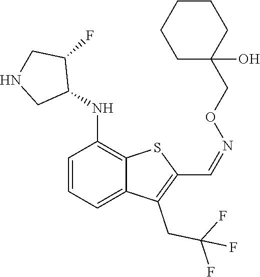 [SMILES: OC1(CO/N=C\c2sc3c(N[C@@H]4CNC[C@@H]4F)cccc3c2CC(F)(F)F)CCCCC1] | 1H NMR (400 MHz, Methanol-d4) δ 8.02 (s, 1H), 7.40 (dq, J = 15.3, 7.7 Hz, 2H), 6.90 (dd, J = 7.3, 1.2 Hz, 1H), 5.44 (dt, J = 53.0, 3.1 Hz, 1H), 4.64 (m, 1H) 4.24 (s, 2H), 4.12-3.94 (m, 2H), 3.89- 3.63 (m, 3H), 3.43 (t, J = 11.2 Hz, 1H), 1.74-1.29 (m, 10H), 19F NMR (376 MHz, Methanol-d4) δ −66.12, −66.22, −76.92, −198.67, −198.68, (M + H)+ 474.10. |
| | (E)-7-(((3R,4S)-4-fluoropyrrolidin-3-yl)amino)-3-(2,2,2- | |
| | trifluoroethyl)benzo[b]thiophene-2-carbaldehyde O-((3- | |
| | hydroxypiperidin-3-yl)methyl) oxime | |
| 1-32 | 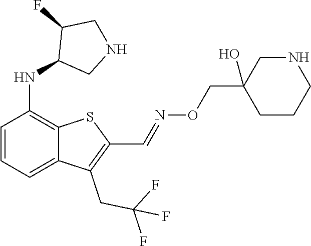 [SMILES: OC1(CO/N=C/c2sc3c(N[C@@H]4CNC[C@@H]4F)cccc3c2CC(F)(F)F)CCCNC1] | 1H NMR (400 MHz, Methanol-d4) δ 8.37 (m, 1H), 7.48-7.33 (m, 2H), 6.95- 6.87 (m, 1H), 5.41 (m 1H), 4.64 (m, 1H), 4.16 (s, 2H), 4.10-3.89 (m, 2H), 3.88-3.63 (m, 3H), 3.46 (t, J = 11.1 Hz, 1H), 3.34 (s, 1H), 3.28-3.19 (m, 1H), 3.12 (d, J = 12.7 Hz, 1H), 2.93 (m, 1H), 2.27-2.09 (m, 1H), 1.96-1.75 (m, 3H), 19F NMR (376 MHz, Methanol- d4) δ −66.09, −66.19, −76.92, −198.36, −198.37, −198.61, (M + H)+ 475.15. |
| | (E)-7-(((3R,4S)-4-fluoropyrrolidin-3-yl)amino)-3-(2,2,2- | |
| | trifluoroethyl)benzo[b]thiophene-2-carbaldehyde O-(3- | |
| | hydroxypiperidin-4-yl) oxime | |
| 1-33 | 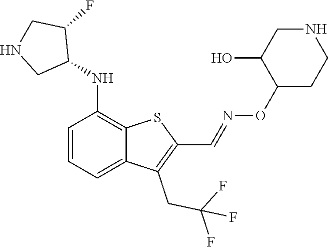 [SMILES: OC1CNCCC1O/N=C/c1sc2c(N[C@@H]3CNC[C@@H]3F)cccc2c1CC(F)(F)F] | 1H NMR (400 MHz, Methanol-d4) δ 8.72 (s, 1H), 7.49-7.35 (m, 2H), 6.96- 6.87 (m, 1H), 5.41 (m, 1H), 4.71-4.56 (m, 1H), 4.39 (m, 1H), 4.22-4.07 (m, 1H), 3.98 (q, J = 10.7 Hz, 2H), 3.90- 3.62 (m, 3H), 3.46 (t, J = 11.1 Hz, 1H), 3.41-3.33 (m, 1H), 3.28-3.10 (m, 3H), 2.41 (m, 1H), 2.09 (d, J = 14.9 Hz, 1H), 19F NMR (376 MHz, Methanol- d4) δ −66.05, −66.09, −66.15, −66.19, −76.91, −198.31, −198.34, −198.40, −198.65, −198.67, (M + H)+ 461.10. |
| | (E)-7-(((3R,4S)-4-fluoropyrrolidin-3-yl)amino)-3-(2,2,2- | |
| | trifluoroethyl)benzo[b]thiophene-2-carbaldehyde O-(3- | |
| | (benzyloxy)-2-hydroxypropyl) oxime | |
| 1-34 | 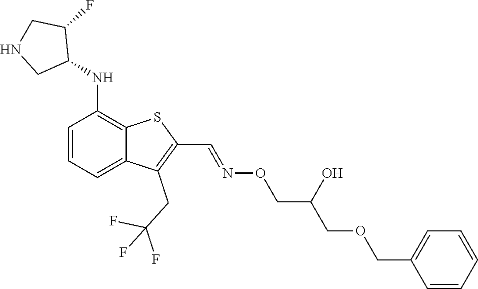 [SMILES: OC(COCc1ccccc1)CO/N=C/c1sc2c(N[C@@H]3CNC[C@@H]3F)cccc2c1CC(F)(F)F] | 1H NMR (400 MHz, Methanol-d4) δ 8.32 (m, 1H), 7.52-7.13 (m, 7H), 6.97- 6.86 (m, 1H), 5.41 (m, 1H), 4.58 (s, 3H), 4.41-4.28 (m, 1H), 4.27-4.16 (m, 1H), 4.14-4.01 (m, 1H), 4.00- 3.65 (m, 5H), 3.65-3.52 (m, 2H), 3.46 (t, J = 11.3 Hz, 1H), 19F NMR (376 MHz, Methanol-d4) δ −66.09, −66.18, −66.20, −76.92, −198.51, −198.56, −198.74, (M + H)+ 526.15. |
| | (E)-7-(((3R,4S)-4-fluoropyrrolidin-3-yl)amino)-3-(2,2,2- | |
| | trifluoroethyl)benzo[b]thiophene-2-carbaldehyde O-(2- | |
| | hydroxycyclohexyl) oxime | |
| 1-35 | 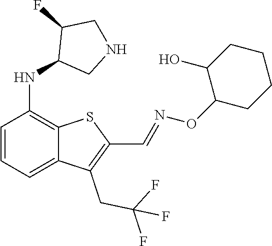 [SMILES: OC1CCCCC1O/N=C/c1sc2c(N[C@@H]3CNC[C@@H]3F)cccc2c1CC(F)(F)F] | 1H NMR (400 MHz, Methanol-d4) δ 8.32 (m, 1H), 7.51-7.25 (m, 2H), 7.01- 6.77 (m, 1H), 5.42 (ddt, J = 52.9, 17.6, 3.4 Hz, 1H), 4.64 (m, 1H), 4.19-4.02 (m, 1H), 3.99-3.88 (m, 2H), 3.87- 3.74 (m, 2H), 3.67 (td, J = 10.7, 10.2, 5.1 Hz, 1H), 3.48 (m, 1H), 2.26-1.95 (m, 2H), 1.89-1.66 (m, 2H), 1.65-1.18 (m, 4H), 19F NMR (376 MHz, Methanol-d4) δ −66.10, −66.21, −76.99, −198.61, −198.63, −198.77, (M + H)+ 460.20. |
| | (E)-7-(((3R,4S)-4-fluoropyrrolidin-3-yl)amino)-3-(2,2,2- | |
| | trifluoroethyl)benzo[b]thiophene-2-carbaldehyde O-(4- | |
| | hydroxypyrrolidin-3-yl) oxime | |
| 1-36 | 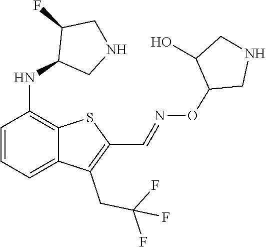 [SMILES: OC1CNCC1O/N=C/c1sc2c(N[C@@H]3CNC[C@@H]3F)cccc2c1CC(F)(F)F] | 1H NMR (400 MHz, Methanol-d4) δ 8.67 (s, 1H), 7.44-7.34 (m, 2H), 6.91 (dd, J = 6.0, 2.7 Hz, 1H), 5.53-5.29 (m, 1H), 4.64 (d, J = 23.1 Hz, 2H), 3.97 (q, J = 10.6 Hz, 2H), 3.88-3.67 (m, 4H), 3.57 (dd, J = 13.2, 3.1 Hz, 1H), 3.51- 3.41 (m, 2H), 3.35 (d, J = 12.3 Hz, 2H), 19F NMR (376 MHz, Methanol-d4) δ −66.10, −66.21, −76.92, −198.33, −198.34, −198.69, (M + H)+ 447.10. |
| | (E)-7-(((3R,4S)-4-fluoropyrrolidin-3-yl)amino)-3-(2,2,2- | |
| | trifluoroethyl)benzo[b]thiophene-2-carbaldehyde O-(4- | |
| | hydroxytetrahydrofuran-3-yl) oxime | |
| 1-37 | 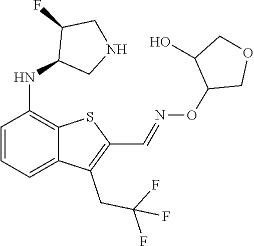 [SMILES: OC1COCC1O/N=C/c1sc2c(N[C@@H]3CNC[C@@H]3F)cccc2c1CC(F)(F)F] | 1H NMR (400 MHz, Methanol-d4) δ 8.39 (m, 1H), 7.53-7.33 (m, 2H), 6.91 (m, 1H), 5.60-5.24 (m, 1H), 4.81- 4.41 (m, 3H), 4.31-3.91 (m, 5H), 3.94- 3.60 (m, 4H), 3.51 m, 1H), 19F NMR (376 MHz, Methanol-d4) δ −66.11, −66.24, −76.90, −198.54, −198.70, −198.73, (M + H)+ 448.15. |
| | (E)-7-(((3R,4S)-4-fluoropyrrolidin-3-yl)amino)-3-(2,2,2- | |
| | trifluoroethyl)benzo[b]thiophene-2-carbaldehyde O-(4-hydroxy- | |
| | 1-(methylsulfonyl)pyrrolidin-3-yl) oxime | |
| 1-38 | 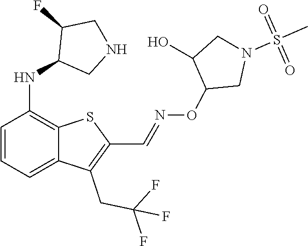 [SMILES: CS(=O)(=O)N1CC(O)C(O/N=C/c2sc3c(N[C@@H]4CNC[C@@H]4F)cccc3c2CC(F)(F)F)C1] | 1H NMR (400 MHz, Methanol-d4) δ 8.41 (d, J = 197.9 Hz, 1H), 7.43-7.28 (m, 2H), 6.89 (dd, J = 13.7, 5.9 Hz, 1H), 5.22 (d, J = 55.5 Hz, 1H), 4.71 (d, J = 4.1 Hz, 1H), 4.59-4.46 (m, 1H), 4.32- 4.17 (m, 1H), 4.02 (m, 2H), 3.85-3.54 (m, 4H), 3.46-3.39 (m, 2H), 3.27- 3.16 (m, 1H), 2.92 (d, J = 7.3 Hz, 4H), 19F NMR (376 MHz, Methanol-d4) δ −66.08, −66.20, −195.39, −195.88, (M + H)+ 525.05. |
| | (E)-7-(((3S,4R)-3-fluoro-1-methylpiperidin-4-yl)amino)-3-(2,2,2- | |
| | trifluoroethyl)benzo[b]thiophene-2-carbaldehyde O-(2-hydroxy- | |
| | 2-methylpropyl) oxime | |
| 1-39 | 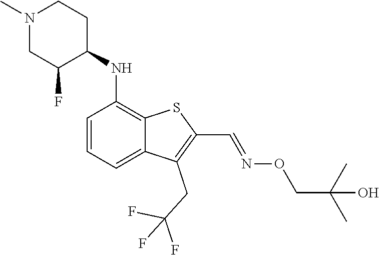 [SMILES: CN1CC[C@@H](Nc2cccc3c(CC(F)(F)F)c(/C=N/OCC(C)(C)O)sc23)[C@@H](F)C1] | 1H NMR (400 MHz, Methanol-d4) δ 8.32 (m, 1H), 7.45-7.27 (m, 2H), 6.94- 6.79 (m, 1H), 5.15 (m, 1H), 4.24- 3.81 (m, 6H), 3.69-3.37 (m, 2H), 3.26 (m, 1H), 2.95 (m, 3H), 2.24 (m, 2H), 1.31 (m, 6H), 19F NMR (376 MHz, Methanol-d4) δ −66.11, −66.21, −66.27, −77.00, −205.13, −205.17, (M + H)+ 462.30. |
| | (E)-7-(((3R,4S)-4-fluoropyrrolidin-3-yl)amino)-3-(2,2,2- | |
| | trifluoroethyl)benzo[b]thiophene-2-carbaldehyde O-((3- | |
| | hydroxytetrahydrofuran-3-yl)methyl) oxime | |
| 1-40 | 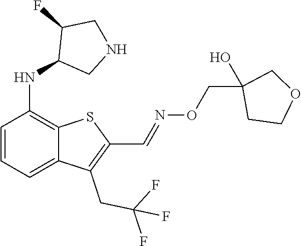 [SMILES: OC1(CO/N=C/c2sc3c(N[C@@H]4CNC[C@@H]4F)cccc3c2CC(F)(F)F)CCOC1] | 1H NMR (400 MHz, Methanol-d4) δ 8.35 (d, J = 232.4 Hz, 1H), 7.50-7.30 (m, 2H), 7.00-6.76 (m, 1H), 5.41 (dt, J = 52.9, 3.2 Hz, 1H), 4.60 (s, 1H), 4.51- 4.22 (m, 2H), 4.14-3.60 (m, 9H), 3.47 (td, J = 11.1, 7.1 Hz, 1H), 2.32-1.86 (m, 2H).19F NMR (376 MHz, Methanol-d4) δ −66.12, −66.22, −76.90, −198.73, −198.75. (M + H)+ 462.10 |

### Example 2-1

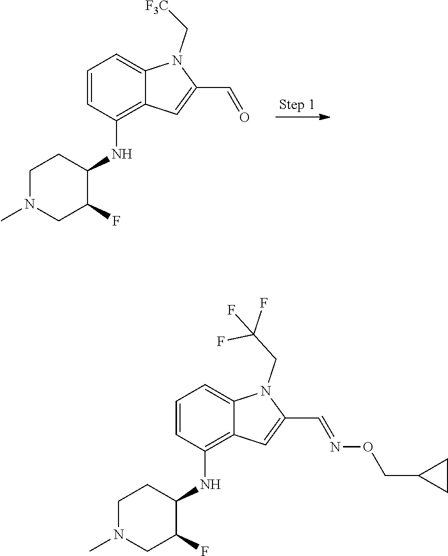 [SMILES: CN1CC[C@@H](Nc2cccc3c2cc(C=O)n3CC(F)(F)F)[C@@H](F)C1 ; CN1CC[C@@H](Nc2cccc3c2cc(/C=N/OCC2CC2)n3CC(F)(F)F)[C@@H](F)C1]

### Step 1. (E)-4-(((3S,4R)-3-fluoro-1-methylpiperidin-4-yl)amino)-1-(2,2,2-trifluoroethyl)-1H-indole-2-carbaldehyde O-cyclopropylmethyl oxime

To a stirred solution of 4-(((3S,4R)-3-fluoro-1-methylpiperidin-4-yl)amino)-1-(2,2,2-trifluoroethyl)-1H-indole-2-carbaldehyde (20 mg, 0.06 mmol) and O-(cyclopropylmethyl) hydroxylamine hydrochloride (10.4 mg, 0.09 mmol) in EtOH (0.4 mL) was added Et₃N (17 mg, 0.2 mmol) dropwise at rt. The resulting mixture was stirred for 3 min at rt. To the above mixture was added AcOH (20.2 mg, 0.4 mmol.) at rt. The resulting mixture was stirred for additional 1 h at rt. The reaction mixture was concentrated under reduced pressure. The residue was purified by reversed-phase flash chromatography with the following conditions: column, C18 silica gel; mobile phase, ACN in water (0.1% NH₃·H₂O), 45% to 60% gradient in 20 min; detector, UV 254 nm. This resulted in (E)-4-(((3S,4R)-3-fluoro-1-methylpiperidin-4-yl)amino)-1-(2,2,2-trifluoroethyl)-1H-indole-2-carbaldehyde O-cyclopropylmethyl oxime (7.5 mg) as a solid.

The compounds in the table below were synthesized as described in Example 1-1 or 2-1 from appropriate intermediates.

| | Chemical name | |
|---|---|---|
| Example | Chemical structure | NMR, MS |
| | (E)-4-(((3S,4R)-3-fluoro-1-methylpiperidin-4-yl)amino)-1-(2,2,2- | |
| | trifluoroethyl)-1H-indole-2-carbaldehyde O-cyclopropylmethyl | |
| | oxime | |
| 2-1 | 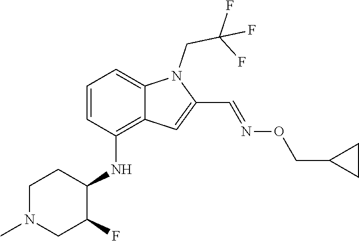 [SMILES: CN1CC[C@@H](Nc2cccc3c2cc(/C=N/OCC2CC2)n3CC(F)(F)F)[C@@H](F)C1] | 1H NMR (300 MHz, Chloroform-d) δ 8.16 (s, 1H), 7.17 (t, J = 8.0 Hz, 1H), 6.77 (d, J = 8.4 Hz, 1H), 6.69 (s, 1H), 6.29 (d, J = 7.7 Hz, 1H), 5.21 (q, J = 8.4 Hz, 2H), 4.87 (d, J = 48.9 Hz, 1H), 4.26 (d, J = 9.7 Hz, 1H), 3.97 (d, J = 7.1 Hz, 2H), 3.58 (d, J = 28.9 Hz, 1H), 3.32-3.20 (m, 1H), 2.97 (d, J = 11.6 Hz, 1H), 2.37 (s, 3H), 2.26-1.94 (m, 3H), 1.20 (ddt, J = 10.1, 7.4, 4.0 Hz, 1H), 0.65-0.53 (m, 2H), 0.32 (q, J = 4.9 Hz, 2H), 19F NMR (282 MHz, CDCl3) δ −69.76, −201.55, (M + H)+ 427.20. |
| | (E)-4-(((3S,4R)-3-fluoro-1-methylpiperidin-4-yl)amino)-1-(2,2,2- | |
| | trifluoroethyl)-1H-indole-2-carbaldehyde O-methyl oxime | |
| 2-2 | 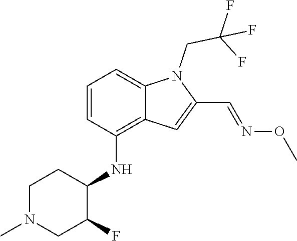 | 1H NMR (400 MHz, Methanol-d4) δ 8.17 (d, J = 1.1 Hz, 1H), 7.13 (t, J = 8.0 Hz, 1H), 6.97 (t, J = 1.1 Hz, 1H), 6.80 (d, J = 8.3 Hz, 1H), 6.36 (d, J = 7.8 Hz, 1H), 5.33 (q, J = 8.7 Hz, 2H), 4.93 (s, 1H), 3.93 (s, 3H), 3.68 (d, J = 31.2 Hz, 1H), 3.21 (t, J = 11.7 Hz, 1H), 2.94 (d, J = 11.7 Hz, 1H), 2.46-2.21 (m, 5H), 2.06-1.91 (m, 2H), 19F NMR (377 MHz, MeOD) δ −71.48, −202.79, (M + H)+ 387.15. |
| | (E)-4-(((3S,4R)-3-fluoro-1-methylpiperidin-4-yl)amino)-1-(2,2,2- | |
| | trifluoroethyl)-1H-indole-2-carbaldehyde O-benzyl oxime | |
| 2-3 | 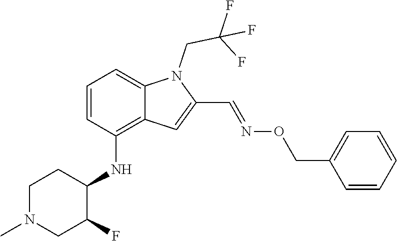 [SMILES: CN1CC[C@@H](Nc2cccc3c2cc(/C=N/OCc2ccccc2)n3CC(F)(F)F)[C@@H](F)C1] | 1H NMR (400 MHz, Methanol-d4) δ 8.24- 8.20 (m, 1H), 7.42-7.26 (m, 5H), 7.11 (t, J = 8.1 Hz, 1H), 6.96 (d, J = 3.2 Hz, 1H), 6.77 (d, J = 8.5 Hz, 1H), 6.34 (d, J = 7.7 Hz, 1H), 5.25 (d, J = 8.0 Hz, 2H), 5.14 (s, 2H), 4.80 (s, 1H), 3.66 (d, J = 30.7 Hz, 1H), 3.19 (t, J = 11.6 Hz, 1H), 2.93 (d, J = 11.7 Hz, 1H), 2.43-2.20 (m, 5H), 2.03- 1.89 (m, 2H), 19F NMR (376 MHz, MeOD) δ −71.39, −202.68, (M + H)+ 463.25. |
| | (Z)-4-(((3S,4R)-3-fluoro-1-methylpiperidin-4-yl)amino)-1-(2,2,2- | |
| | trifluoroethyl)-1H-indole-2-carbaldehyde O-benzyl oxime | |
| 2-4 | 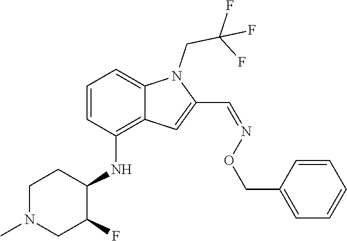 [SMILES: CN1CC[C@@H](Nc2cccc3c2cc(/C=N\OCc2ccccc2)n3CC(F)(F)F)[C@@H](F)C1] | 1H NMR (400 MHz, Methanol-d4) δ 7.80 (s, 1H), 7.68 (s, 1H), 7.45 (d, J = 7.2 Hz, 2H), 7.36 (t, J = 7.5 Hz, 2H), 7.30 (t, J = 7.2 Hz, 1H), 7.16 (t, J = 8.0 Hz, 1H), 6.82 (d, J = 8.4 Hz, 1H), 6.37 (d, J = 7.8 Hz, 1H), 5.33 (s, 2H), 5.06 (q, J = 8.8 Hz, 2H), 4.80 (s, 1H), 3.66 (dt, J = 28.9, 7.6 Hz, 1H), 3.24-3.15 (m, 1H), 2.92 (d, J = 11.8 Hz, 1H), 2.40-2.17 (m, 5H), 1.95 (td, J = 10.3, 9.6, 3.8 Hz, 2H), 19F NMR (376 MHz, MeOD) δ −72.09, −202.77, (M + H)+ 463.20. |
| | (Z)-4-(((3S,4R)-3-fluoro-1-methylpiperidin-4-yl)amino)-1-(2,2,2- | |
| | trifluoroethyl)-1H-indole-2-carbaldehyde O-phenyl oxime | |
| 2-5 | 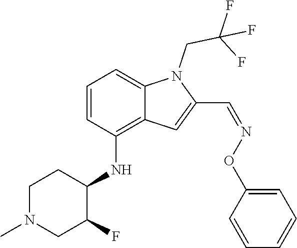 [SMILES: CN1CC[C@@H](Nc2cccc3c2cc(/C=N\Oc2ccccc2)n3CC(F)(F)F)[C@@H](F)C1] | 1H NMR (400 MHz, Methanol-d4) δ 8.05 (s, 1H), 7.97 (s, 1H), 7.51-7.28 (m, 4H), 7.19 (t, J = 8.1 Hz, 1H), 7.07 (t, J = 7.2 Hz, 1H), 6.84 (d, J = 8.4 Hz, 1H), 6.38 (d, J = 7.7 Hz, 1H), 5.15 (q, J = 8.8 Hz, 2H), 4.99 (s, 1H), 3.72 (d, J = 21.8 Hz, 1H), 3.22 (d, J = 11.0 Hz, 1H), 2.97 (d, J = 11.2 Hz, 1H), 2.52-2.19 (m, 6H), 2.08-1.93 (m, 2H), 19F NMR (376 MHz, MeOD) δ −72.04, −202.43, (M + H)+ 449.20. |
| | (E)-4-(((3S,4R)-3-fluoro-1-methylpiperidin-4-yl)amino)-1-(2,2,2- | |
| | trifluoroethyl)-1H-indole-2-carbaldehyde O-phenyl oxime | |
| 2-6 | 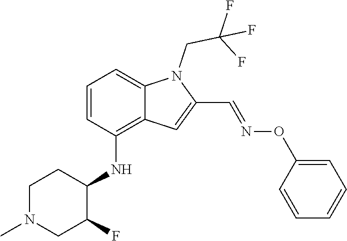 [SMILES: CN1CC[C@@H](Nc2cccc3c2cc(/C=N/Oc2ccccc2)n3CC(F)(F)F)[C@@H](F)C1] | 1H NMR (400 MHz, Chloroform-d) δ 8.48 (s, 1H), 7.39-7.33 (m, 2H), 7.24-7.17 (m, 3H), 7.07 (dd, J = 8.0, 6.7 Hz, 1H), 6.85 (d, J = 9.2 Hz, 2H), 6.33 (d, J = 7.7 Hz, 1H), 5.29 (q, J = 8.4 Hz, 2H), 4.96 (s, 1H), 4.30 (s, 1H), 3.72 (s, 1H), 3.45 (s, 1H), 3.21 (s, 1H), 2.60 (s, 5H), 2.19 (s, 2H), 19F NMR (377 MHz, CDCl3) δ −69.65, −200.95, (M + H)+ 449.20. |
| | 2-((((E)-(4-(((3S,4R)-3-fluoro-1-methylpiperidin-4-yl)amino)-1- | |
| | (2,2,2-trifluoroethyl)-1H-indol-2-yl)methylene)amino)oxy)-N- | |
| | methylacetamide | |
| 2-7 | 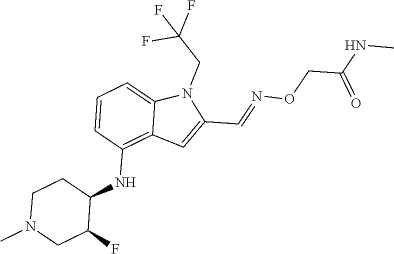 | 1H NMR (400 MHz, Methanol-d4) δ 8.41 (d, J = 2.4 Hz, 1H), 8.35 (d, J = 1.6 Hz, 1H), 7.17 (td, J = 8.3, 2.0 Hz, 1H), 7.09 (s, 1H), 6.83 (d, J = 8.3 Hz, 1H), 6.39 (d, J = 7.7 Hz, 1H), 5.28 (q, J = 8.7 Hz, 2H), 5.14- 4.94 (m, 1H), 4.57 (s, 2H), 3.83 (dt, J = 28.4, 8.3 Hz, 1H), 3.52 (d, J = 11.9 Hz, 1H), 3.24 (d, J = 12.1 Hz, 1H), 2.97-2.80 (m, 4H), 2.71 (dd, J = 13.8, 9.8 Hz, 1H), 2.59 (d, J = 2.2 Hz, 3H), 2.22-2.00 (m, 2H), 19F NMR (376 MHz, MeOD) δ −71.49, −72.08, −203.60, (M + H)+ 444.20. |
| | 2-((((E)-(4-(((3S,4R)-3-fluoro-1-methylpiperidin-4-yl)amino)-1- | |
| | (2,2,2-trifluoroethyl)-1H-indol-2-yl)methylene)amino)oxy)-N- | |
| | phenylacetamide | |
| 2-8 | 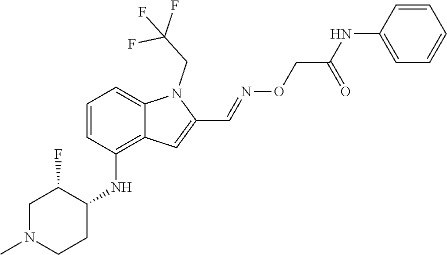 [SMILES: CN1CC[C@@H](Nc2cccc3c2cc(/C=N/OCC(=O)Nc2ccccc2)n3CC(F)(F)F)[C@@H](F)C1] | 1H NMR (400 MHz, Methanol-d4) δ 8.41 (s, 1H), 7.58 (d, J = 7.9 Hz, 2H), 7.31 (t, J = 7.8 Hz, 2H), 7.21-7.05 (m, 3H), 6.78 (d, J = 8.3 Hz, 1H), 6.35 (d, J = 7.7 Hz, 1H), 5.26 (q, J = 8.7 Hz, 2H), 4.96-4.92 (m, 1H), 4.85-4.82 (m, 1H), 4.72 (s, 2H), 3.75-3.61 (m, 1H), 3.22 (t, J = 11.5 Hz, 1H), 2.95 (d, J = 11.9 Hz, 1H), 2.42 (d, J = 13.3 Hz, 1H), 2.32 (s, 3H), 2.26 (d, J = 12.4 Hz, 1H), 2.06-1.90 (m, 2H), 19F NMR (377 MHz, MeOD) δ −71.39, −202.75, (M + H)+ 506.10. |
| | (E)-4-(((3S,4R)-3-fluoro-1-methylpiperidin-4-yl)amino)-1-(2,2,2- | |
| | trifluoroethyl)-1H-indole-2-carbaldehyde O-((1H- | |
| | benzo[d]imidazol-2-yl)methyl)oxime | |
| 2-9 | 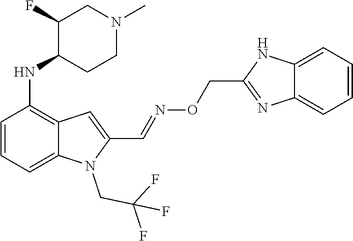 [SMILES: CN1CC[C@@H](Nc2cccc3c2cc(/C=N/OCc2nc4ccccc4[nH]2)n3CC(F)(F)F)[C@@H](F)C1] | 1H NMR (400 MHz, Methanol-d4) δ 8.36 (s, 1H), 7.57 (dd, J = 6.0, 3.2 Hz, 2H), 7.24 (dd, J = 6.1, 3.2 Hz, 2H), 7.12 (t, J = 8.0 Hz, 1H), 7.05 (d, J = 0.8 Hz, 1H), 6.75 (d, J = 8.4 Hz, 1H), 6.35 (d, J = 7.8 Hz, 1H), 5.39 (s, 2H), 5.19 (q, J = 8.7 Hz, 2H), 4.96- 4.79 (m, 1H), 3.75-3.60 (m, 1H), 3.25- 3.16 (m, 1H), 2.99-2.90 (m, 1H), 2.45- 2.35 (m, 1H), 2.32 (s, 3H), 2.26-2.20 (m, 1H), 2.04-1.94 (m, 2H), 19F NMR (377 MHz, MeOD) δ −72.10, −202.74, (M + H)+ 503.15. |
| | (E)-7-(((3S,4R)-3-fluoropiperidin-4-yl)amino)-3-(2,2,2- | |
| | trifluoroethyl)benzo[b]thiophene-2-carbaldehyde O- | |
| | cyclopropylmethyl oxime | |
| 2-10 | 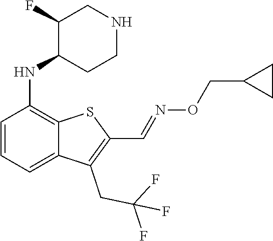 [SMILES: F[C@H]1CNCC[C@H]1Nc1cccc2c(CC(F)(F)F)c(/C=N/OCC3CC3)sc12] | 1H NMR (400 MHz, Methanol-d4) δ 8.55 (s, 1H), 7.37-7.29 (m, 2H), 6.86 (m, 1H), 5.13 (d, J = 47.0 Hz, 1H), 4.17-3.87 (m, 5H), 3.78-3.69 (m, 1H), 3.54-3.36 (m, 2H), 3.29-3.22 (m, 1H), 2.24-2.10 (m, 2H), 1.21 (m, 1H), 0.63-0.54 (m, 2H), 0.39-0.31 (m, 2H), 19F NMR (376 MHz, MeOD) δ −66.23, −76.95, −206.51, (M + H)+ 430.10. |
| | (Z)-7-(((3S,4R)-3-fluoropiperidin-4-yl)amino)-3-(2,2,2- | |
| | trifluoroethyl)benzo[b]thiophene-2-carbaldehyde O- | |
| | cyclopropylmethyl oxime | |
| 2-11 | 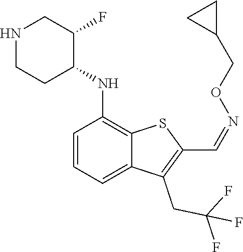 [SMILES: F[C@H]1CNCC[C@H]1Nc1cccc2c(CC(F)(F)F)c(/C=N\OCC3CC3)sc12] | 1H NMR (400 MHz, Methanol-d4) δ 8.01 (s, 1H), 7.43-7.32 (m, 2H), 6.88 (m, 1H), 5.17 (m, 1H), 4.15 (m, 2H), 4.10-3.98 (m, 3H), 3.76 (m, 1H), 3.53-3.37 (m, 2H), 3.24 (m, 1H), 2.28-2.13 (m, 2H), 1.37-1.26 (m, 1H), 0.66-0.58 (m, 2H), 0.41 (m, 2H), 19F NMR (376 MHz, MeOD) δ −66.11, −76.94, −206.42, (M + H)+ 430.10. |
| | (E)-7-(((3S,4R)-3-fluoropiperidin-4-yl)amino)-3-(2,2,2- | |
| | trifluoroethyl)benzo[b]thiophene-2-carbaldehyde O-(2-hydroxy- | |
| | 2-phenylethyl) oxime | |
| 2-12 | 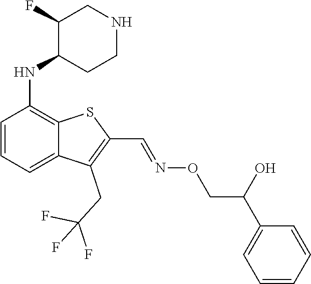 [SMILES: OC(CO/N=C/c1sc2c(N[C@@H]3CCNC[C@@H]3F)cccc2c1CC(F)(F)F)c1ccccc1] | 1H NMR (400 MHz, Methanol-d4) δ 8.32 (m, 1H), 7.51-7.41 (m, 2H), 7.40-7.25 (m, 5H), 6.82 (m, 1H), 5.11-4.98 (m, 1H), 4.74 (m, 1H), 4.54-4.35 (m, 1H), 4.31-4.21 (m, 1H), 4.09-3.76 (m, 3H), 3.27 (m, 1H), 3.16-3.04 (m, 1H), 2.97- 2.65 (m, 2H), 2.00-1.90 (m, 1H), 1.82 (m, 1H), 19F NMR (376 MHz, MeOD) δ −66.16, −206.52, (M + H)+ 469.20. |
| | (E)-7-(((3S,4R)-3-fluoropiperidin-4-yl)amino)-3-(2,2,2- | |
| | trifluoroethyl)benzo[b]thiophene-2-carbaldehyde O-(2- | |
| | hydroxypropyl) oxime | |
| 2-13 | 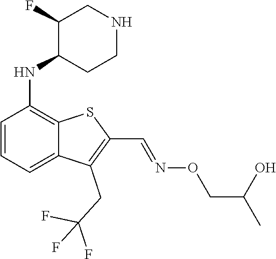 [SMILES: CC(O)CO/N=C/c1sc2c(N[C@@H]3CCNC[C@@H]3F)cccc2c1CC(F)(F)F] | 1H NMR (400 MHz, Methanol-d4) δ 8.55 (d, J = 28.2 Hz, 1H), 7.37-7.25 (m, 2H), 6.84 (dd, J = 5.6, 3.1 Hz, 1H), 4.97 (dd, J = 48.3, 12.5 Hz, 1H), 4.27-4.13 (m, 1H), 4.12-3.84 (m, 5H), 3.52 (t, J = 12.4 Hz, 1H), 3.21 (dd, J = 14.3, 4.8 Hz, 1H), 3.11 (dd, J = 14.2, 4.9 Hz, 1H), 3.06-2.94 (m, 1H), 2.13-1.92 (m, 2H), 1.27 (d, J = 6.2 Hz, 1H), 1.23-1.17 (m, 2H), 19F NMR (377 MHz, MeOD) δ −66.23, −206.71, (M + H)+ 434.10. |
| | (E)-7-(((3S,4R)-3-fluoro-1-methylpiperidin-4-yl)amino)-3-(2,2,2- | |
| | trifluoroethyl)benzo[b]thiophene-2-carbaldehyde O-methyl | |
| | oxime | |
| 2-14 | 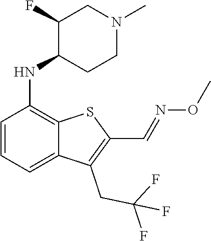 [SMILES: CO/N=C/c1sc2c(N[C@@H]3CCN(C)C[C@@H]3F)cccc2c1CC(F)(F)F] | 1H NMR (400 MHz, Methanol-d4) δ 8.52 (s, 1H), 7.35-7.25 (m, 2H), 6.85-6.78 (m, 1H), 4.92 (d, J = 12.8 Hz, 1H), 4.11 (s, 1H), 4.03 (q, J = 10.7 Hz, 1H), 3.97 (s, 2H), 3.91 (q, J = 10.7 Hz, 1H), 3.83-3.70 (m, 1H), 3.22 (s, 1H), 2.94 (dd, J = 11.7, 2.8 Hz, 1H), 2.47-2.21 (m, 5H), 1.97 (tt, J = 9.5, 4.8 Hz, 2H), 19F NMR (377 MHz, MeOD) δ −66.12, −66.24, −202.95, (M + H)+ 404.20. |
| | (E)-7-(((3S,4R)-3-fluoro-1-methylpiperidin-4-yl)amino)-3-(2,2,2- | |
| | trifluoroethyl)benzo[b]thiophene-2-carbaldehyde O-((1H- | |
| | benzo[d]imidazol-2-yl)methyl) oxime | |
| 2-15 | 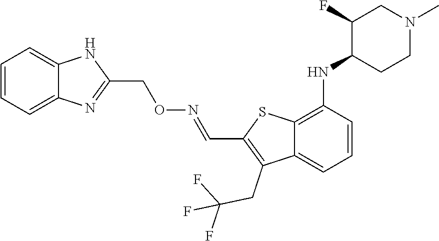 [SMILES: CN1CC[C@@H](Nc2cccc3c(CC(F)(F)F)c(/C=N/OCc4nc5ccccc5[nH]4)sc23)[C@@H](F)C1] | 1H NMR (400 MHz, Methanol-d4) δ 8.72 (d, J = 1.9 Hz, 1H), 7.57 (s, 2H), 7.36- 7.30 (m, 1H), 7.26 (dtd, J = 10.1, 6.9, 2.8 Hz, 3H), 6.80 (tt, J = 6.6, 2.2 Hz, 1H), 5.56- 5.41 (m, 2H), 4.76 (s, 1H), 4.08-3.87 (m, 2H), 3.79-3.65 (m, 1H), 3.19 (t, J = 11.8 Hz, 1H), 2.91 (d, J = 12.3 Hz, 1H), 2.29 (dd, J = 7.4, 2.0 Hz, 5H), 1.93 (q, J = 7.3, 6.7 Hz, 2H), 19F NMR (376 MHz, MeOD) δ −66.08, −66.17, −202.97, −203.04, (M + H)+ 521.20. |
| | (E)-7-(((3R,4S)-4-fluoropyrrolidin-3-yl)amino)-3-(2,2,2- | |
| | trifluoroethyl)benzo[b]thiophene-2-carbaldehyde O-(tert-butyl) | |
| | oxime | |
| 2-16 | 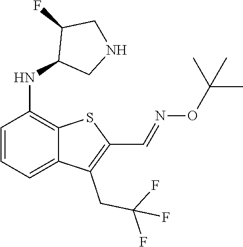 [SMILES: CC(C)(C)O/N=C/c1sc2c(N[C@@H]3CNC[C@@H]3F)cccc2c1CC(F)(F)F] | 1H NMR (400 MHz, Methanol-d4) δ 8.24 (d, J = 186.6 Hz, 1H), 7.45-7.30 (m, 2H), 6.87 (m, 1H), 5.42 (m, 1H), 4.64 (m, 1H), 3.98 (m, 2H), 3.86-3.61 (m, 3H), 3.47 (m, 1H), 1.42 (d, J = 40.6 Hz, 9H), 19F NMR (376 MHz, MeOD) δ −66.11, −66.24, −76.93, −198.70, −198.74, (M + H)+ 418.05. |
| | (E)-7-(((3R,4S)-4-fluoropyrrolidin-3-yl)amino)-3-(2,2,2- | |
| | trifluoroethyl)benzo[b]thiophene-2-carbaldehyde O-cyclopentyl | |
| | oxime | |
| 2-17 | 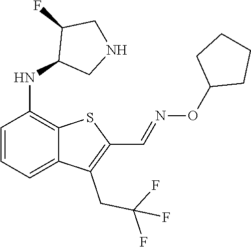 [SMILES: F[C@H]1CNC[C@H]1Nc1cccc2c(CC(F)(F)F)c(/C=N/OC3CCCC3)sc12] | 1H NMR (400 MHz, Methanol-d4) δ 8.24 (d, J = 191.4 Hz, 1H), 7.40-7.23 (m, 2H), 6.89-6.80 (m, 1H), 5.34-5.13 (m, 1H), 4.92 (m, 1H), 4.26 (m, 1H), 3.97 (m, 2H), 3.49-3.35 (m, 2H), 3.29-3.17 (m, 1H), 2.91 (m, 1H), 2.06 (d, J = 16.5 Hz, 1H), 1.94-1.82 (m, 4H), 1.78-1.58 (m, 3H), 19F NMR (376 MHz, MeOD) δ −66.09, −66.22, −76.94, −196.13, −196.29, (M + H)+ 430.00. |
| | (E)-7-(((3R,4S)-4-fluoropyrrolidin-3-yl)amino)-3-(2,2,2- | |
| | trifluoroethyl)benzo[b]thiophene-2-carbaldehyde O-((5- | |
| | oxopyrrolidin-2-yl)methyl) oxime | |
| 2-18 | 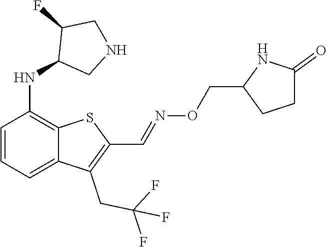 [SMILES: O=C1CCC(CO/N=C/c2sc3c(N[C@@H]4CNC[C@@H]4F)cccc3c2CC(F)(F)F)N1] | 1H NMR (400 MHz, Methanol-d4) δ 8.62 (s, 1H), 7.38-7.25 (m, 2H), 6.85 (td, J = 7.3, 3.0 Hz, 1H), 5.21 (ddt, J = 55.4, 8.0, 4.0 Hz, 1H), 4.42-3.87 (m, 6H), 3.44- 3.32 (m, 2H), 3.27-3.13 (m, 1H), 3.04- 2.82 (m, 1H), 2.51-2.24 (m, 3H), 2.19- 1.90 (m, 1H), 19F NMR (377 MHz, MeOD) δ −66.10, −66.19, −195.67, −195.69, −195.87, −195.87, (M + H)+ 459.10. |
| | (E)-7-(((3R,4S)-4-fluoropyrrolidin-3-yl)amino)-3-(2,2,2- | |
| | trifluoroethyl)benzo[b]thiophene-2-carbaldehyde O-((2- | |
| | oxooxazolidin-4-yl)methyl) oxime | |
| 2-19 | 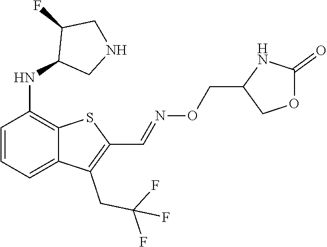 [SMILES: O=C1NC(CO/N=C/c2sc3c(N[C@@H]4CNC[C@@H]4F)cccc3c2CC(F)(F)F)CO1] | 1H NMR (400 MHz, Chloroform-d) δ 8.43 (d, J = 4.5 Hz, 1H), 7.36 (dt, J = 12.7, 8.0 Hz, 1H), 7.22 (d, J = 8.0 Hz, 1H), 6.72 (t, J = 8.6 Hz, 1H), 5.19 (d, J = 54.7 Hz, 2H), 4.63-4.49 (m, 1H), 4.46-4.33 (m, 1H), 4.29-4.10 (m, 4H), 3.81-3.64 (m, 2H), 3.48 (dd, J = 11.0, 6.6 Hz, 1H), 3.39-3.21 (m, 1H), 3.05-2.87 (m, 1H), 19F NMR (376 MHz, CDCl3) δ −64.45, −194.79, (M + H)+ 461.10. |
| | (E)-7-(((3R,4S)-4-fluoropyrrolidin-3-yl)amino)-3-(2,2,2- | |
| | trifluoroethyl)benzo[b]thiophene-2-carbaldehyde O-((5-methyl- | |
| | 2-oxo-1,3-dioxol-4-yl)methyl) oxime | |
| 2-20 | 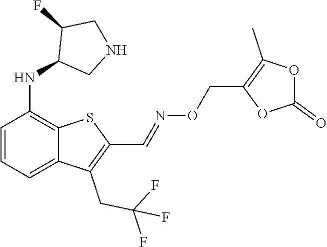 [SMILES: Cc1oc(=O)oc1CO/N=C/c1sc2c(N[C@@H]3CNC[C@@H]3F)cccc2c1CC(F)(F)F] | 1H NMR (400 MHz, Methanol-d4) δ 8.40 (m, 1H), 7.52-7.31 (m, 2H), 6.91 (dt, J = 8.0, 5.9 Hz, 1H), 5.44 (m, 1H), 5.06 (m, 2H), 4.75-4.54 (m, 1H), 4.21-3.63 (m, 5H), 3.61-3.44 (m, 1H), 2.26 (m, 3H), 19F NMR (376 MHz, Methanol-d4) δ −66.13, −66.24, −77.06, −198.64, −198.72, (M + H)+ 474.10. |
| | (E)-7-(((3R,4S)-4-fluoro-1-methylpyrrolidin-3-yl)amino)-3- | |
| | (2,2,2-trifluoroethyl)benzo[b]thiophene-2-carbaldehyde O-(2- | |
| | hydroxy-2-methylpropyl) oxime | |
| 2-21 | 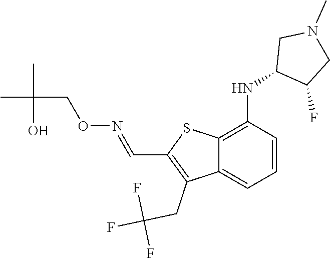 [SMILES: CN1C[C@H](F)[C@H](Nc2cccc3c(CC(F)(F)F)c(/C=N/OCC(C)(C)O)sc23)C1] | 1H NMR (400 MHz, Methanol-d4) δ 8.35 (m, 1H), 7.49-7.33 (m, 2H), 6.93-6.84 (m, 1H), 5.49 (m, 1H), 4.77 (m, 1H), 4.29- 4.06 (m, 3H), 3.96 (m, 3H), 3.81-3.49 (m, 2H), 3.12-2.90 (m, 3H), 1.33 (d, J = 31.0 Hz, 6H), 19F NMR (376 MHz, Methanol-d4) δ −66.14, −66.24, −76.94, −199.60, M + H: 448.15. |
| | (E)-7-(((3R,4S)-4-fluoro-1-isopropylpyrrolidin-3-yl)amino)-3- | |
| | (2,2,2-trifluoroethyl)benzo[b]thiophene-2-carbaldehyde O-(2- | |
| | hydroxy-2-methylpropyl) oxime | |
| 2-22 | 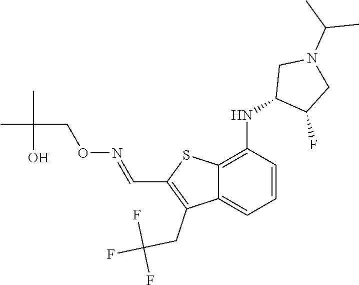 [SMILES: CC(C)N1C[C@H](F)[C@H](Nc2cccc3c(CC(F)(F)F)c(/C=N/OCC(C)(C)O)sc23)C1] | 1H NMR (400 MHz, Methanol-d4) δ 8.33 (m, 1H), 7.48-7.31 (m, 2H), 6.89 (s, 1H), 5.48 (d, J = 49.8 Hz, 1H), 4.57 (s, 1H), 4.24-4.03 (m, 3H), 3.93 (dd, J = 12.5, 9.1 Hz, 2H), 3.55 (m, 4H), 1.43 (dt, J = 6.4, 2.7 Hz, 6H), 1.31 (d, J = 30.8 Hz, 6H), 19F NMR (376 MHz, Methanol-d4) δ −66.14, −66.24, −77.00, −193.21, −199.78, M + H: 476.15. |
| | (E)-7-(((3R,4S)-4-fluoropyrrolidin-3-yl)amino)-3-(2,2,2- | |
| | trifluoroethyl)benzo[b]thiophene-2-carbaldehyde O-((2- | |
| | oxooxazolidin-5-yl)methyl) oxime | |
| 2-23 | 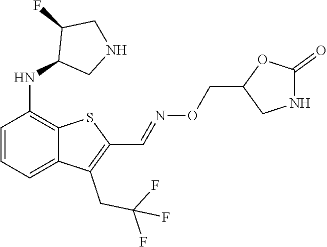 [SMILES: O=C1NCC(CO/N=C/c2sc3c(N[C@@H]4CNC[C@@H]4F)cccc3c2CC(F)(F)F)O1] | 1H NMR (400 MHz, Methanol-d4) δ 8.36 (m, 1H), 7.40-7.26 (m, 2H), 6.85 (m, 1H), 5.36-5.13 (m, 1H), 5.08-4.94 (m, 1H), 4.63-4.33 (m, 2H), 4.31-4.13 (m, 1H), 3.98 (m, 2H), 3.75 (m, 1H), 3.58 (m, 1H), 3.42-3.32 (m, 2H), 3.27-3.11 (m, 1H), 3.08-2.78 (m, 1H). 19F NMR (376 MHz, Methanol-d4) δ −66.10, −66.20, −195.55, −195.75, −195.86. (M + H)+ 461.05 |
| | (E)-7-(((3R,4S)-4-fluoropyrrolidin-3-yl)amino)-3-(2,2,2- | |
| | trifluoroethyl)benzo[b]thiophene-2-carbaldehyde O-cyclobutyl | |
| | oxime | |
| 2-24 | 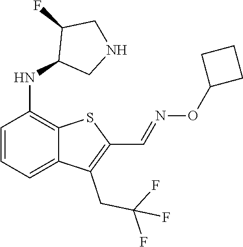 [SMILES: F[C@H]1CNC[C@H]1Nc1cccc2c(CC(F)(F)F)c(/C=N/OC3CCC3)sc12] | 1H NMR (400 MHz, Methanol-d4) δ 8.31 (m, 1H), 7.48-7.30 (m, 2H), 6.94-6.86 (m, 1H), 5.44 (m, 1H), 4.94 (s, 1H), 4.73- 4.57 (m, 1H), 4.01 (m, 2H), 3.89-3.76 (m, 2H), 3.73-3.45 (m, 2H), 2.45-2.16 (m, 4H), 1.94-1.62 (m, 2H). 19F NMR (376 MHz, Methanol-d4) δ −66.13, −66.26, −77.06, −198.62, −198.78. (M + H)+ 416.10 |
| | (E)-7-(((3R,4S)-4-fluoropyrrolidin-3-yl)amino)-3-(2,2,2- | |
| | trifluoroethyl)benzo[b]thiophene-2-carbaldehyde O-(1-(pyridin- | |
| | 3-yl)ethyl) oxime | |
| 2-25 | 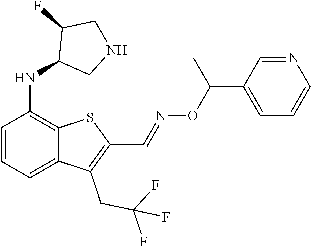 [SMILES: CC(O/N=C/c1sc2c(N[C@@H]3CNC[C@@H]3F)cccc2c1CC(F)(F)F)c1cccnc1] | 1H NMR (400 MHz, Methanol-d4) δ 8.92- 8.64 (m, 3H), 8.43 (m, 1H), 7.96-7.77 (m, 1H), 7.56-7.31 (m, 2H), 7.00-6.84 (m, 1H), 5.64 (m, 1H), 5.51-5.30 (m, 1H), 4.77-4.50 (m, 1H), 4.19-3.91 (m, 2H), 3.90-3.61 (m, 3H), 3.59-3.41 (m, 1H), 1.79 (m, 3H). 19F NMR (376 MHz, Methanol-d4) δ −66.12, −66.21, −77.14, −198.51, −198.55, −198.74, −198.75. (M + H)+ 467.15 |
| | (E)-7-(((3R,4S)-4-fluoropyrrolidin-3-yl)amino)-3-(2,2,2- | |
| | trifluoroethyl)benzo[b]thiophene-2-carbaldehyde O-((6-oxo-1,6- | |
| | dihydropyridin-2-yl)methyl) oxime | |
| 2-26 | 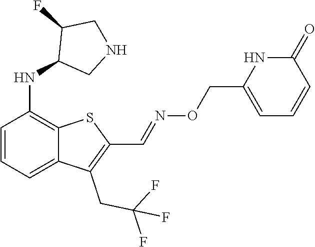 [SMILES: O=c1cccc(CO/N=C/c2sc3c(N[C@@H]4CNC[C@@H]4F)cccc3c2CC(F)(F)F)[nH]1] | 1H NMR (400 MHz, Methanol-d4) δ 8.43 (d, J = 223.0 Hz, 1H), 7.62-7.55 (m, 1H), 7.48-7.32 (m, 2H), 6.95-6.86 (m, 1H), 6.54-6.44 (m, 2H), 5.49-5.30 (m, 1H), 5.14 (d, J = 66.1 Hz, 2H), 4.72-4.54 (m, 1H), 4.15-3.89 (m, 2H), 3.86-3.62 (m, 3H), 3.52-3.41 (m, 1H). 19F NMR (376 MHz, Methanol-d4) δ −66.12, −77.15, −198.73. (M + H)+ 469.10 |
| | (E)-7-(((3S,4R)-3-fluoro-1-methylpiperidin-4-yl)amino)-3-(2,2,2- | |
| | trifluoroethyl)benzo[b]thiophene-2-carbaldehyde O-((6- | |
| | methoxypyridin-2-yl)methyl) oxime | |
| 2-27 | 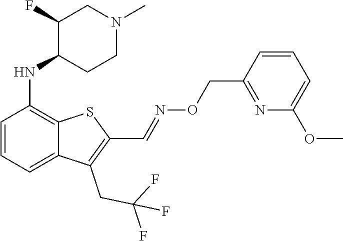 [SMILES: COc1cccc(CO/N=C/c2sc3c(N[C@@H]4CCN(C)C[C@@H]4F)cccc3c2CC(F)(F)F)n1] | 1H NMR (400 MHz, Methanol-d4) δ 8.72 (s, 1H), 7.67 (dd, J = 8.3, 7.3 Hz, 1H), 7.45- 7.31 (m, 2H), 7.04 (t, J = 7.8 Hz, 1H), 6.86 (dd, J = 6.4, 2.1 Hz, 1H), 6.75-6.69 (m, 1H), 5.24-5.05 (m, 3H), 4.08-3.84 (m, 7H), 3.60 (d, J = 13.0 Hz, 1H), 3.46 (dd, J = 38.4, 14.1 Hz, 1H), 3.25 (d, J = 12.3 Hz, 1H), 2.94 (d, J = 4.2 Hz, 3H), 2.23 (td, J = 9.7, 9.0, 3.4 Hz, 2H). 19F NMR (376 MHz, Methanol-d4) δ −66.22, −77.28, −205.22. (M + H)+ 511.20 |
| | (Z)-7-(((3S,4R)-3-fluoro-1-methylpiperidin-4-yl)amino)-3-(2,2,2- | |
| | trifluoroethyl)benzo[b]thiophene-2-carbaldehyde O-((6- | |
| | methoxypyridin-2-yl)methyl) oxime | |
| 2-28 | 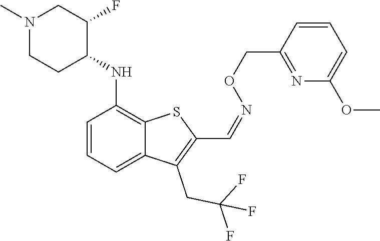 | 1H NMR (400 MHz, Methanol-d4) δ 8.10 (s, 1H), 7.66 (q, J = 7.8 Hz, 1H), 7.46- 7.32 (m, 2H), 7.05 (d, J = 7.4 Hz, 1H), 6.88 (d, J = 7.3 Hz, 1H), 6.71 (dd, J = 8.4, 3.8 Hz, 1H), 5.36 (s, 2H), 5.21 (d, J = 9.4 Hz, 1H), 4.08 (p, J = 10.5, 9.7 Hz, 3H), 3.92 (t, J = 1.9 Hz, 4H), 3.62-3.40 (m, 3H), 2.94 (s, 3H), 2.31-2.20 (m, 2H). 19F NMR (376 MHz, Methanol-d4) δ −66.12, −77.29, −77.80, −205.0. (M + H)+ 511.15 |
| | (E)-7-(((3S,4R)-3-fluoro-1-methylpiperidin-4-yl)amino)-3-(2,2,2- | |
| | trifluoroethyl)benzo[b]thiophene-2-carbaldehyde O-((1-methyl- | |
| | 6-oxo-1,6-dihydropyridin-2-yl)methyl) oxime | |
| 2-29 | 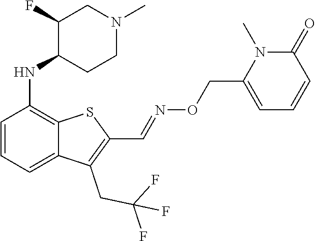 [SMILES: CN1CC[C@@H](Nc2cccc3c(CC(F)(F)F)c(/C=N/OCc4cccc(=O)n4C)sc23)[C@@H](F)C1] | 1H NMR (400 MHz, Methanol-d4) δ 8.72 (s, 1H), 7.55-7.32 (m, 3H), 6.95-6.83 (m, 1H), 6.63-6.52 (m, 2H), 5.25 (s, 2H), 5.13 (d, J = 46.8 Hz, 1H), 4.12-3.83 (m, 4H), 3.75-3.40 (m, 5H), 3.25 (d, J = 12.2 Hz, 1H), 2.95 (d, J = 4.8 Hz, 3H), 2.24 (s, 2H). 19F NMR (376 MHz, Methanol-d4) δ −66.22, −77.26, −205.17. (M + H)+ 511.15 |
| | (Z)-7-(((3S,4R)-3-fluoro-1-methylpiperidin-4-yl)amino)-3-(2,2,2- | |
| | trifluoroethyl)benzo[b]thiophene-2-carbaldehyde O-((1-methyl- | |
| | 6-oxo-1,6-dihydropyridin-2-yl)methyl) oxime | |
| 2-30 | 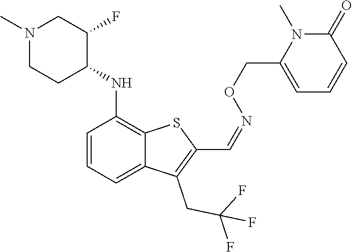 [SMILES: CN1CC[C@@H](Nc2cccc3c(CC(F)(F)F)c(/C=N\OCc4cccc(=O)n4C)sc23)[C@@H](F)C1] | 1H NMR (400 MHz, Methanol-d4) δ 8.14 (s, 1H), 7.59-7.18 (m, 3H), 6.90 (d, J = 7.6 Hz, 1H), 6.66-6.45 (m, 2H), 5.40 (d, J = 1.3 Hz, 2H), 5.13 (d, J = 46.7 Hz, 1H), 4.17-3.83 (m, 4H), 3.72 (d, J = 1.2 Hz, 2H), 3.68-3.34 (m, 4H), 3.23 (s, 1H), 2.95 (s, 3H), 2.24 (s, 2H). 19F NMR (376 MHz, Methanol-d4) δ −66.11, −77.31, −205.11. (M + H)+ 511.15 |
| | (E)-7-(((3S,4R)-3-fluoro-1-methylpiperidin-4-yl)amino)-3-(2,2,2- | |
| | trifluoroethyl)benzo[b]thiophene-2-carbaldehyde O-((6-oxo-1,6- | |
| | dihydropyridin-2-yl)methyl) oxime | |
| 2-31 | 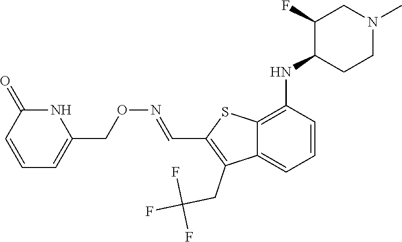 [SMILES: CN1CC[C@@H](Nc2cccc3c(CC(F)(F)F)c(/C=N/OCc4cccc(=O)[nH]4)sc23)[C@@H](F)C1] | 1H NMR (400 MHz, Methanol-d4) δ 8.74- 8.11 (m, 1H), 7.59 (ddd, J = 9.1, 6.9, 2.0 Hz, 1H), 7.48-7.30 (m, 2H), 6.95-6.85 (m, 1H), 6.49 (td, J = 9.7, 9.3, 6.5 Hz, 2H), 5.25-5.05 (m, 3H), 4.15-3.83 (m, 4H), 3.70-3.34 (m, 3H), 2.95 (d, J = 6.8 Hz, 3H), 2.25 (d, J = 12.5 Hz, 2H), 19F NMR (377 MHz, Methanol-d4) δ −66.12, −66.23, −77.08, −77.59, −205.19, M + H: 497.2. |
| | (E)-7-(((3S,4R)-3-fluoro-1-methylpiperidin-4-yl)amino)-3- | |
| | ((trifluoromethyl)thio)benzo[b]thiophene-2-carbaldehyde O-(2- | |
| | hydroxy-2-methylpropyl) oxime | |
| 2-32 | 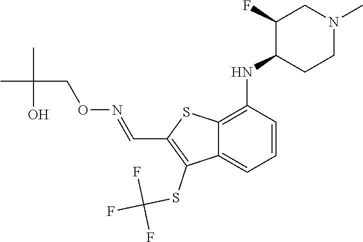 [SMILES: CN1CC[C@@H](Nc2cccc3c(SC(F)(F)F)c(/C=N/OCC(C)(C)O)sc23)[C@@H](F)C1] | 1H NMR (400 MHz, Methanol-d4) δ 8.54 (m, 1H), 7.57-7.32 (m, 2H), 6.88 (m, 1H), 5.00-4.752 (m, 1H), 4.20 (m, 2H), 3.87-3.67 (m, 1H), 3.27-3.15 (m, 1H), 2.99-2.90 (m, 1H), 2.47-2.22 (m, 5H), 1.99 (m, 2H), 1.31 (m, 6H), 19F NMR (377 MHz, Methanol-d4) δ −43.97, −43.99, −202.78, M + H: 480.10. |
| | 4-(((((E)-(7-(((3S,4R)-3-fluoro-1-methylpiperidin-4-yl)amino)-3- | |
| | ((trifluoromethyl)thio)benzo[b]thiophen-2- | |
| | yl)methylene)amino)oxy)methyl)-N-methyl-3- | |
| | (methylamino)benzamide | |
| 2-33 | 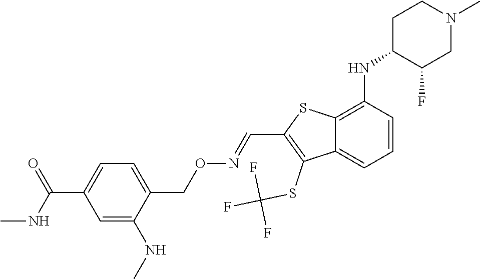 | 1H NMR (400 MHz, Methanol-d4) δ 8.75- 8.34 (m, 1H), 7.54-7.24 (m, 3H), 7.13- 7.05 (m, 2H), 6.91-6.83 (m, 1H), 5.31 (d, J = 65.8 Hz, 2H), 4.95-4.77 (m, 1H), 3.85- 3.66 (m, 1H), 3.27-3.15 (m, 1H), 3.10- 2.70 (m, 7H), 2.45-2.17 (m, 5H), 2.07- 1.91 (m, 2H), 19F NMR (377 MHz, Methanol-d4) δ −43.94, −43.97, −202.59, −202.76, M + H: 584.20. |
| | 4-(((((E)-(7-(((3S,4R)-3-fluoro-1-methylpiperidin-4-yl)amino)-3- | |
| | ((trifluoromethyl)thio)benzofuran-2- | |
| | yl)methylene)amino)oxy)methyl)-N-methyl-3- | |
| | (methylamino)benzamide | |
| 2-34 | 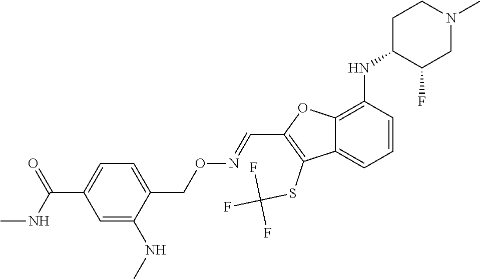 | 1H NMR (400 MHz, DMSO-d6) δ 8.48 (s, 1H), 8.35-8.22 (m, 1H), 7.28-7.16 (m, 2H), 7.08-7.03 (m, 1H), 7.03-6.99 (m, 1H), 6.95 (d, J = 7.7 Hz, 1H), 6.88 (d, J = 7.9 Hz, 1H), 5.55-5.41 (m, 2H), 5.26 (s, 2H), 4.93-4.72 (m, 1H), 3.85-3.67 (m, 1H), 3.11-3.00 (m, 1H), 2.87-2.71 (m, 7H), 2.31-2.14 (m, 4H), 2.13-2.04 (m, 1H), 1.96-1.72 (m, 2H), 19F NMR (377 MHz, DMSO-d6) δ −41.95, −199.88, M + H: 568.15. |

### Example 3-1

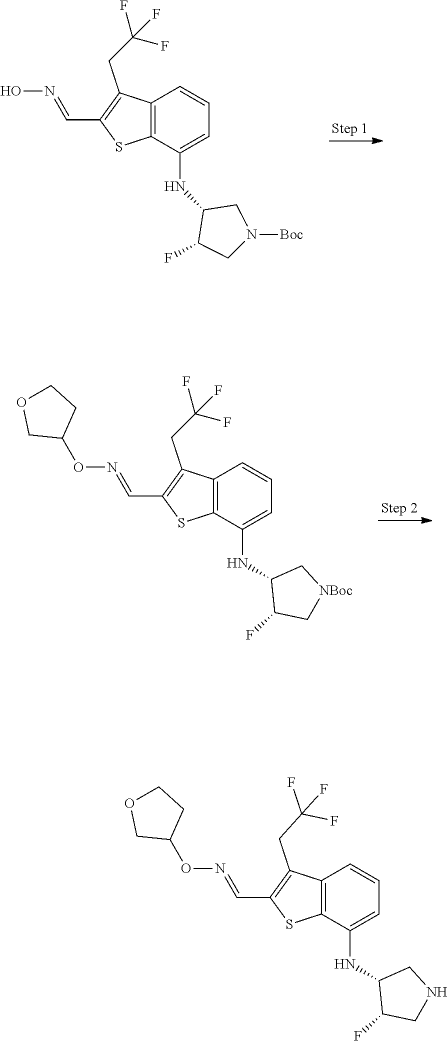 [SMILES: CC(C)(C)OC(=O)N1C[C@H](F)[C@H](Nc2cccc3c(CC(F)(F)F)c(/C=N/O)sc23)C1 ; CC(C)(C)OC(=O)N1C[C@H](F)[C@H](Nc2cccc3c(CC(F)(F)F)c(/C=N/OC4CCOC4)sc23)C1 ; F[C@H]1CNC[C@H]1Nc1cccc2c(CC(F)(F)F)c(/C=N/OC3CCOC3)sc12]

### Step 1. Tert-butyl (3S,4R)-3-fluoro-4-((2-((E)-(((tetrahydrofuran-3-yl)oxy)imino)methyl)-3-(2,2,2-trifluoroethyl)benzo[b]thiophen-7-yl)amino)pyrrolidine-1-carboxylate

To a stirred mixture of tert-butyl (3S,4R)-3-fluoro-4-((2-((E)-(hydroxyimino)methyl)-3-(2,2,2-trifluoroethyl)benzo[b]thiophen-7-yl)amino)pyrrolidine-1-carboxylate (40 mg, 0.09 mmol) and Cs₂CO₃ (42.4 mg, 0.1 mmol) in ACN (1 mL) was added 3-iodotetrahydrofuran (26 mg, 0.1 mmol) at rt. The resulting mixture was stirred for 8 h at rt. The reaction mixture was concentrated under reduced pressure. The residue was purified by reversed-phase flash chromatography with the following conditions: column, C18 silica gel; mobile phase, ACN in water (0.1% TFA), 30% to 60% gradient in 15 min; detector, UV 254 nm. This resulted in tert-butyl (3S,4R)-3-fluoro-4-((2-((E)-(((tetrahydrofuran-3-yl)oxy)imino)methyl)-3-(2,2,2-trifluoroethyl)benzo[b]thiophen-7-yl)amino)pyrrolidine-1-carboxylate (25 mg) as a solid. LCMS [M+H]⁺ m/z: 532.2.

### Step 2. (E)-7-(((3R,4S)-4-fluoropyrrolidin-3-yl)amino)-3-(2,2,2-trifluoroethyl)benzo[b]thiophene-2-carbaldehyde O-tetrahydrofuran-3-yl oxime

To a stirred solution of tert-butyl (3S,4R)-3-fluoro-4-((2-((E)-(((tetrahydrofuran-3-yl)oxy)imino)methyl)-3-(2,2,2-trifluoroethyl)benzo[b]thiophen-7-yl)amino)pyrrolidine-1-carboxylate (20 mg, 0.04 mmol) in DCM (0.8 mL) was added TFA (0.2 mL) at 0° C. The resulting mixture was stirred for 2 h at rt. The reaction mixture was concentrated under reduced pressure. The crude product was purified by Prep-HPLC with the following conditions: Column: Sunfire Prep C18 column, 30*150 mm, 5 μm; Mobile Phase A: Water (0.05% TFA), Mobile Phase B: MeOH-HPLC; Flow rate: 60 mL/min; Gradient: isocratic 30% B to 48% B in 10 min; Wave Length: 254 nm/220 nm nm; RT1 (min): 11.5. This resulted in (E)-7-(((3R,4S)-4-fluoropyrrolidin-3-yl)amino)-3-(2,2,2-trifluoroethyl)benzo[b]thiophene-2-carbaldehyde O-tetrahydrofuran-3-yl oxime (11.6 mg) as a solid.

The compounds in the table below were synthesized as described in Example 3-1 from appropriate intermediates.

| | Chemical name | |
|---|---|---|
| Example | Chemical structure | NMR, MS |
| | (E)-7-(((3R,4S)-4-fluoropyrrolidin-3-yl)amino)-3-(2,2,2- | |
| | trifluoroethyl)benzo[b]thiophene-2-carbaldehyde O- | |
| | tetrahydrofuran-3-yl oxime | |
| 3-1 | 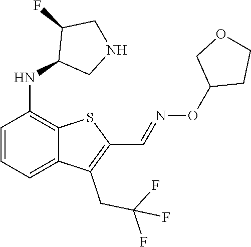 [SMILES: F[C@H]1CNC[C@H]1Nc1cccc2c(CC(F)(F)F)c(/C=N/OC3CCOC3)sc12] | 1H NMR (400 MHz, Methanol-d4) δ 8.34 (m, 1H), 7.45-7.31 (m, 2H), 6.94-6.83 (m, 1H), 5.54-5.32 (m, 1H), 5.05 (m, 1H), 4.72-4.56 (m, 1H), 4.17-3.67 (m, 9H), 3.49 (m, 1H), 2.40-2.15 (m, 2H), 19F NMR (376 MHz, Methanol-d4) δ −66.12, −66.25, −77.02, −198.61, −198.63, −198.76, (M + H)+ 432.10. |
| | (E)-7-(((3R,4S)-4-fluoropyrrolidin-3-yl)amino)-3-(2,2,2- | |
| | trifluoroethyl)benzo[b]thiophene-2-carbaldehyde O-pyridin-3- | |
| | ylmethyl oxime | |
| 3-2 | 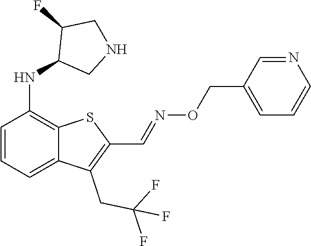 [SMILES: F[C@H]1CNC[C@H]1Nc1cccc2c(CC(F)(F)F)c(/C=N/OCc3cccnc3)sc12] | 1H NMR (400 MHz, Methanol-d4) δ 8.80 (s, 1H), 8.73 (s, 1H), 8.68 (d, J = 5.6 Hz, 1H), 8.32 (d, J = 7.9 Hz, 1H), 7.83-7.73 (m, 1H), 7.48-7.35 (m, 2H), 6.96-6.85 (m, 1H), 5.51 (d, J = 18.7 Hz, 1H), 5.37 (d, J = 9.4 Hz, 2H), 4.72-4.54 (m, 1H), 4.03 (dq, J = 45.9, 10.7 Hz, 2H), 3.89- 3.62 (m, 3H), 3.50 (q, J = 10.9 Hz, 1H), 19F NMR (376 MHz, MeOD) δ −66.23, −77.11, −198.74, (M + H)+ 453.10. |
| | (E)-7-(((3R,4S)-4-fluoropyrrolidin-3-yl)amino)-3-(2,2,2- | |
| | trifluoroethyl)benzo[b]thiophene-2-carbaldehyde O-pyridin-2- | |
| | ylmethyl oxime | |
| 3-3 | 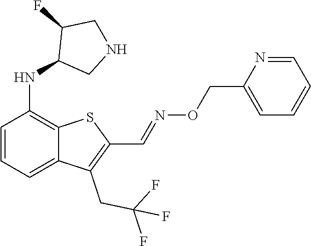 [SMILES: F[C@H]1CNC[C@H]1Nc1cccc2c(CC(F)(F)F)c(/C=N/OCc3ccccn3)sc12] | 1H NMR (400 MHz, Methanol-d4) δ 8.72 (s, 1H), 8.55 (d, J = 4.9 Hz, 1H), 7.93- 7.85 (m, 1H), 7.60 (dd, J = 16.5, 7.9 Hz, 1H), 7.43-7.27 (m, 3H), 6.90-6.82 (m, 1H), 5.40 (d, J = 62.8 Hz, 2H), 5.21 (dt, J = 55.4, 4.0 Hz, 1H), 4.30-4.15 (m, 1H), 4.01 (dq, J = 46.2, 10.6 Hz, 2H), 3.40 (dd, J = 10.1, 7.4 Hz, 1H), 3.29-3.14 (m, 2H), 2.89 (dt, J = 11.1, 8.5 Hz, 1H), 19F NMR (376 MHz, MeOD) δ −66.09, −195.77, (M + H)+ 453.10. |
| | (E)-7-(((3R,4S)-4-fluoropyrrolidin-3-yl)amino)-3-(2,2,2- | |
| | trifluoroethyl)benzo[b]thiophene-2-carbaldehyde O-((1-methyl- | |
| | 1H-1,2,4-triazol-3-yl)methyl) oxime | |
| 3-4 | 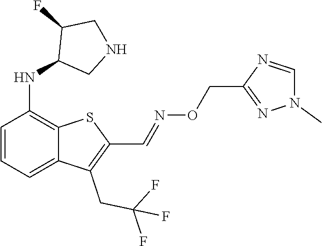 [SMILES: Cn1cnc(CO/N=C/c2sc3c(N[C@@H]4CNC[C@@H]4F)cccc3c2CC(F)(F)F)n1] | 1H NMR (400 MHz, Methanol-d4) δ 8.60 (s, 1H), 8.39 (d, J = 2.8 Hz, 1H), 7.35- 7.25 (m, 2H), 6.83 (d, J = 7.2 Hz, 1H), 5.35-5.16 (m, 3H), 4.27-4.13 (m, 1H), 3.94 (d, J = 2.3 Hz, 3H), 3.89 (d, J = 10.7 Hz, 2H), 3.36 (m, 2H), 3.17 (m, 1H), 2.91- 2.83 (m, 1H), 19F NMR (376 MHz, MeOD) δ −66.11, −66.22, −195.64, −195.84, (M + H)+ 457.14. |
| | (E)-7-(((3R,4S)-4-fluoropyrrolidin-3-yl)amino)-3-(2,2,2- | |
| | trifluoroethyl)benzo[b]thiophene-2-carbaldehyde O- | |
| | (benzo[d]thiazol-2-ylmethyl) oxime | |
| 3-5 | 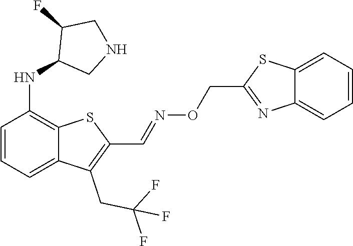 [SMILES: F[C@H]1CNC[C@H]1Nc1cccc2c(CC(F)(F)F)c(/C=N/OCc3nc4ccccc4s3)sc12] | 1H NMR (400 MHz, Methanol-d4) δ 8.84 (s, 1H), 8.03 (d, J = 8.1 Hz, 2H), 7.57 (td, J = 7.5, 7.1, 1.2 Hz, 1H), 7.52-7.45 (m, 1H), 7.45-7.35 (m, 2H), 6.98-6.87 (m, 1H), 5.71 (d, J = 61.4 Hz, 2H), 5.56- 5.28 (m, 1H), 4.77-4.52 (m, 1H), 4.16- 3.96 (m, 2H), 3.91-3.62 (m, 3H), 3.51 (dt, J = 20.2, 11.1 Hz, 1H), 19F NMR (376 MHz, MeOD) δ −66.09, −77.11, −198.73, (M + H)+ 509.05. |
| | (E)-7-(((3R,4S)-4-fluoropyrrolidin-3-yl)amino)-3-(2,2,2- | |
| | trifluoroethyl)benzo[b]thiophene-2-carbaldehyde O-((5-methyl- | |
| | 1,3,4-oxadiazol-2-yl)methyl) oxime | |
| 3-6 | 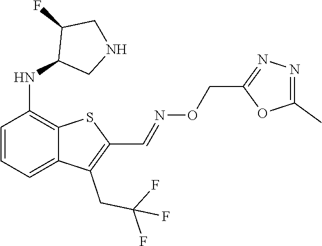 [SMILES: Cc1nnc(CO/N=C/c2sc3c(N[C@@H]4CNC[C@@H]4F)cccc3c2CC(F)(F)F)o1] | 1H NMR (400 MHz, Methanol-d4) δ 8.71 (s, 1H), 7.49-7.27 (m, 2H), 6.95-6.82 (m, 1H), 5.45 (d, J = 55.4 Hz, 2H), 5.31- 5.15 (m, 1H), 4.33-4.18 (m, 1H), 4.10- 3.89 (m, 2H), 3.52-3.39 (m, 2H), 3.27- 3.14 (m, 1H), 2.97-2.85 (m, 1H), 2.59 (d, J = 2.3 Hz, 3H), 19F NMR (376 MHz, MeOD) δ −66.10, −195.70, (M + H)+ 458.05. |
| | (E)-7-(((3R,4S)-4-fluoropyrrolidin-3-yl)amino)-3-(2,2,2- | |
| | trifluoroethyl)benzo[b]thiophene-2-carbaldehyde O-((5- | |
| | methylisoxazol-3-yl)methyl) oxime | |
| 3-7 | 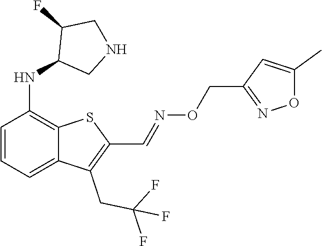 [SMILES: Cc1cc(CO/N=C/c2sc3c(N[C@@H]4CNC[C@@H]4F)cccc3c2CC(F)(F)F)no1] | 1H NMR (400 MHz, Methanol-d4) δ 8.36 (d, J = 219.2 Hz, 1H), 7.40-7.23 (m, 2H), 6.85 (m, 1H), 6.26 (d, J = 1.7 Hz, 1H), 5.40-5.10 (m, 3H), 4.21 (m, 1H), 3.98 (m, 2H), 3.47-3.33 (m, 2H), 3.25- 3.15 (m, 1H), 2.94-2.82 (m, 1H), 2.42 (d, J = 1.0 Hz, 3H), 19F NMR (376 MHz, MeOD) δ −66.11, −66.22, −195.76, −195.84, (M + H)+ 457.05. |
| | (E)-7-(((3R,4S)-4-fluoropyrrolidin-3-yl)amino)-3-(2,2,2- | |
| | trifluoroethyl)benzo[b]thiophene-2-carbaldehyde O-((5- | |
| | oxopyrrolidin-3-yl)methyl) oxime | |
| 3-8 | 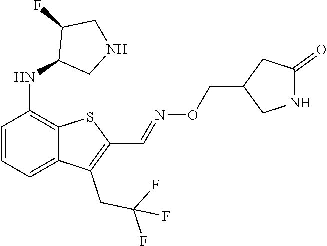 [SMILES: O=C1CC(CO/N=C/c2sc3c(N[C@@H]4CNC[C@@H]4F)cccc3c2CC(F)(F)F)CN1] | 1H NMR (400 MHz, Methanol-d4) δ 8.34 (d, J = 213.2 Hz, 1H), 7.47-7.30 (m, 2H), 6.90 (m, 1H), 5.53-5.31 (m, 1H), 4.74-4.57 (m, 1H), 4.30 (d, J = 63.3, 5.8 Hz, 2H), 4.00 (d, J = 46.4, 10.6 Hz, 2H), 3.86-3.37 (m, 6H), 3.08-2.90 (m, 1H), 2.57-2.45 (m, 1H), 2.27 (d, J = 17.1, 6.5 Hz, 1H), 19F NMR (376 MHz, Methanol- d4) δ −66.15, −66.25, −76.95, −198.55, −198.66, −198.77, (M + H)+ 446.15. |

### Example 4-1

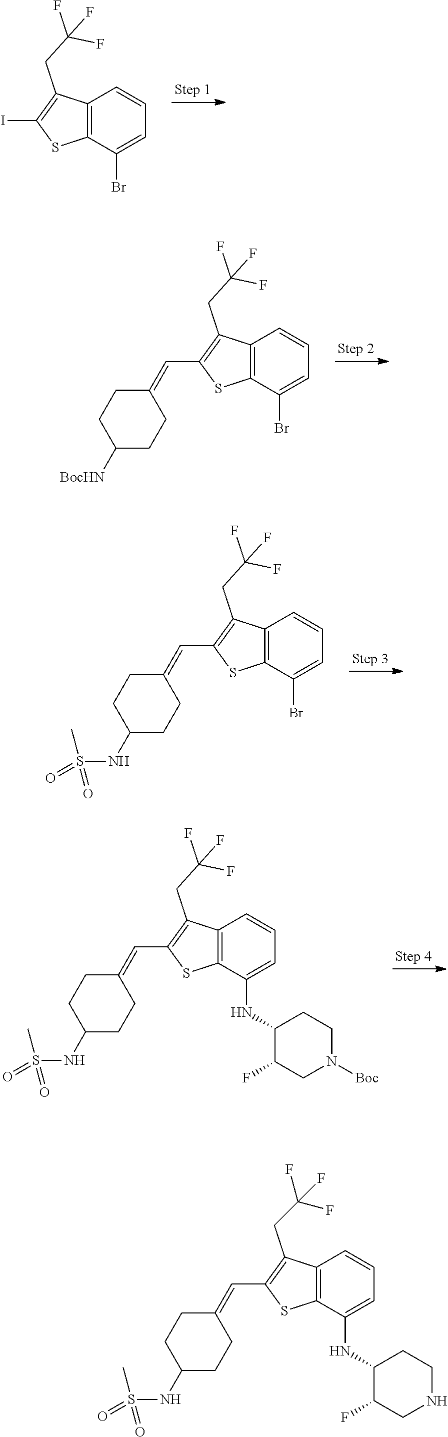 [SMILES: FC(F)(F)Cc1c(I)sc2c(Br)cccc12 ; CC(C)(C)OC(=O)NC1CCC(=Cc2sc3c(Br)cccc3c2CC(F)(F)F)CC1 ; CS(=O)(=O)NC1CCC(=Cc2sc3c(Br)cccc3c2CC(F)(F)F)CC1 ; CC(C)(C)OC(=O)N1CC[C@@H](Nc2cccc3c(CC(F)(F)F)c(C=C4CCC(NS(C)(=O)=O)CC4)sc23)[C@@H](F)C1 ; CS(=O)(=O)NC1CCC(=Cc2sc3c(N[C@@H]4CCNC[C@@H]4F)cccc3c2CC(F)(F)F)CC1]

### Step 1. Tert-butyl (4-((7-bromo-3-(2,2,2-trifluoroethyl)benzo[b]thiophen-2-yl)methylene)cyclohexyl)carbamate

A mixture of 7-bromo-2-iodo-3-(2,2,2-trifluoroethyl)benzo[b]thiophene (500 mg, 1.2 mmol), tert-butyl (4-((4,4,5,5-tetramethyl-1,3,2-dioxaborolan-2-yl)methylene)cyclohexyl)carbamate (CAS no. 2694028-09-4, 400.5 mg, 1.2 mmol), Pd(dppf)Cl₂·CH₂Cl₂ (96.8 mg, 0.1 mmol) and Cs₂CO₃ (1.1 g, 3.6 mmol) in dioxane (5 mL) and H₂O (1 mL) was stirred for 2 h at 60° C. under nitrogen atmosphere. The resulting mixture was concentrated under reduced pressure. The residue was purified by silica gel column chromatography, eluted with PE/EA (10:1) to afford tert-butyl (4-((7-bromo-3-(2,2,2-trifluoroethyl)benzo[b]thiophen-2-yl)methylene)cyclohexyl)carbamate (220 mg) as a solid. LCMS [M+H]⁺ m/z: 504.3.

### Step 2. N-(4-((7-bromo-3-(2,2,2-trifluoroethyl)benzo[b]thiophen-2-yl)methylene)cyclohexyl)methanesulfonamide

To a stirred solution of tert-butyl (4-((7-bromo-3-(2,2,2-trifluoroethyl)benzo[b]thiophen-2-yl)methylene)cyclohexyl)carbamate (210 mg, 0.4 mmol) in DCM (2 mL) was added TFA (0.4 mL) dropwise at 0° C. under air atmosphere. The resulting mixture was stirred for 1 h at rt. The resulting mixture was concentrated under reduced pressure. The residue was dissolved in DCM (2 mL), then Et₃N (0.3 mL, 2 mmol) was added at 0° C. The resulting mixture was stirred for additional 15 min at rt. MsCl (62 mg, 0.5 mmol) in DCM (0.2 mL) was added into above solution at 0° C. The resulting mixture was stirred for additional 1 h at rt. The resulting mixture was concentrated under reduced pressure. The residue was purified by silica gel column chromatography, eluted with PE/EA (1:1) to afford N-(4-((7-bromo-3-(2,2,2-trifluoroethyl)benzo[b]thiophen-2-yl)methylene)cyclohexyl)methanesulfonamide (180 mg) as a solid. LCMS [M+H]⁺ m/z: 482.1.

### Step 3. Tert-butyl (3S,4R)-3-fluoro-4-((2-((4-(methylsulfonamido)cyclohexylidene)methyl)-3-(2,2,2-trifluoroethyl)benzo[b]thiophen-7-yl)amino)piperidine-1-carboxylate

A stirred mixture of N-(4-((7-bromo-3-(2,2,2-trifluoroethyl)benzo[b]thiophen-2-yl)methylene)cyclohexyl)methanesulfonamide (100 mg, 0.2 mmol), tert-butyl (3S,4R)-4-amino-3-fluoropiperidine-1-carboxylate (54.3 mg, 0.2 mmol), XPhos (9.9 mg, 0.02 mmol), XPhos Pd G3 (17.5 mg, 0.02 mmol) and Cs₂CO₃ (202.6 mg, 0.6 mmol) in THF (3 mL) was stirred for 1 h at 100° C. under nitrogen atmosphere. The resulting mixture was concentrated under reduced pressure. The residue was purified by silica gel column chromatography, eluted with PE/EA (1:1) to afford tert-butyl (3S,4R)-3-fluoro-4-((2-((4-(methylsulfonamido)cyclohexylidene)methyl)-3-(2,2,2-trifluoroethyl)benzo[b]thiophen-7-yl)amino)piperidine-1-carboxylate (70 mg) as a solid. LCMS [M+H]⁺ m/z: 620.3.

### Step 4. N-(4-((7-(((3S,4R)-3-fluoropiperidin-4-yl)amino)-3-(2,2,2-trifluoroethyl)benzo[b]thiophen-2-yl)methylene)cyclohexyl)methanesulfonamide

To a stirred solution of tert-butyl (3S,4R)-3-fluoro-4-((2-((4-(methylsulfonamido)cyclohexylidene)methyl)-3-(2,2,2-trifluoroethyl)benzo[b]thiophen-7-yl)amino)piperidine-1-carboxylate (50 mg, 0.08 mmol) in DCM (2 mL) was added TFA (0.5 mL) at 0° C. The resulting mixture was stirred for 1 h at rt. The resulting mixture was basified to pH 8 with sat. NaHCO₃ (aq.). The aqueous layer was extracted with EA (3×10 mL). The combined organic layers were washed with brine (10 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in water (10 mmol/L FA), 10% to 50% gradient in 25 min; detector, UV 254 nm. This resulted in N-(4-((7-(((3S,4R)-3-fluoropiperidin-4-yl)amino)-3-(2,2,2-trifluoroethyl)benzo[b]thiophen-2-yl)methylene)cyclohexyl)methanesulfonamide (16 mg) as a solid.

The compounds in the table below were synthesized as described in Example 4-1 from appropriate intermediates.

| | Chemical name | |
|---|---|---|
| | Chemical | |
| Example | structure | NMR, MS |
| | N-(4-((7-(((3S,4R)-3-fluoropiperidin-4-yl)amino)-3-(2,2,2- | |
| | trifluoroethyl)benzo[b]thiophen-2- | |
| | yl)methylene)cyclohexyl)methanesulfonamide | |
| 4-1 | 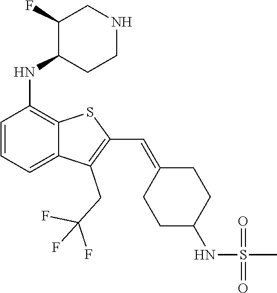 [SMILES: CS(=O)(=O)NC1CCC(=Cc2sc3c(N[C@@H]4CCNC[C@@H]4F)cccc3c2CC(F)(F)F)CC1] | 1H NMR (400 MHz, Methanol-d4) δ 8.51 (s, 1H), 7.29 (t, J = 7.8 Hz, 1H), 7.22 (d, J = 8.0 Hz, 1H), 6.77 (d, J = 7.5 Hz, 1H), 6.43 (s, 1H), 5.11- 4.99 (m, 1H), 4.08-3.94 (m, 1H), 3.78-3.59 (m, 3H), 3.57-3.47 (m, 1H), 3.43-3.32 (m, 2H), 3.25 (d, J = 14.3 Hz, 1H), 3.19-3.07 (m, 1H), 2.97 (s, 4H), 2.59-2.50 (m, 1H), 2.46- 2.35 (m, 1H), 2.29-2.21 (m, 1H), 2.19-2.05 (m, 4H), 1.62-1.39 (m, 2H)., 19F NMR (377 MHz, MeOD) δ −65.820, −206.528, (M + H)+ 520.25. |
| | 1-(4-((7-(((3S,4R)-3-fluoropiperidin-4-yl)amino)-3-(2,2,2- | |
| | trifluoroethyl)benzo[b]thiophen-2-yl)methylene)piperidin-1- | |
| | yl)ethan-1-one | |
| 4-2 | 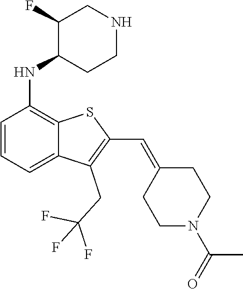 [SMILES: CC(=O)N1CCC(=Cc2sc3c(N[C@@H]4CCNC[C@@H]4F)cccc3c2CC(F)(F)F)CC1] | 1H NMR (400 MHz, Methanol-d4) δ 7.34-7.23 (m, 2H), 6.79 (d, J = 7.5 Hz, 1H), 6.62 (d, J = 5.9 Hz, 1H), 5.14 (d, J = 47.0 Hz, 1H), 4.15-4.00 (m, 1H), 3.80-3.62 (m, 6H), 3.58 (t, J = 5.9 Hz, 1H), 3.49 (d, J = 14.2 Hz, 2H), 3.39 (d, J = 14.3 Hz, 1H), 3.28-3.19 (m, 1H), 2.74-2.62 (m, 2H), 2.58-2.45 (m, 2H), 2.19 (d, J = 7.6 Hz, 2H), 2.15 (d, J = 5.8 Hz, 3H), 19F NMR (377 MHz, MeOD) δ −65.800, −77.232, −206.473, (M + H)+ 470.25. |

### Example 5-1

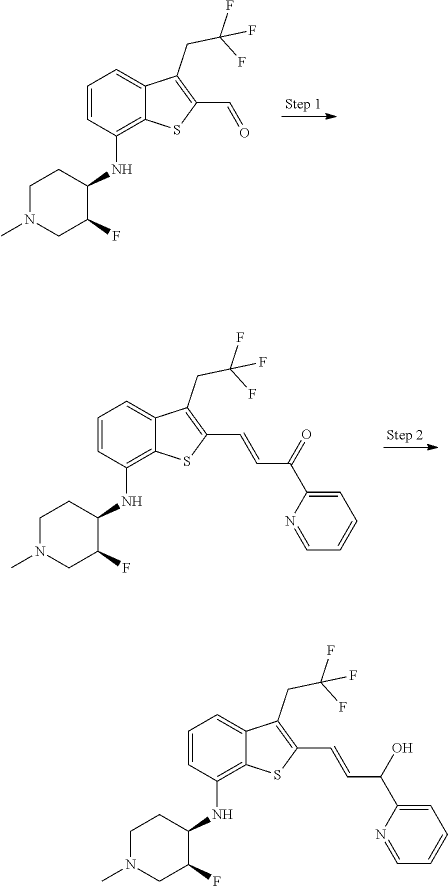 [SMILES: CN1CC[C@@H](Nc2cccc3c(CC(F)(F)F)c(C=O)sc23)[C@@H](F)C1 ; CN1CC[C@@H](Nc2cccc3c(CC(F)(F)F)c(/C=C/C(=O)c4ccccn4)sc23)[C@@H](F)C1 ; CN1CC[C@@H](Nc2cccc3c(CC(F)(F)F)c(/C=C/C(O)c4ccccn4)sc23)[C@@H](F)C1]

### Step 1. (E)-3-(7-(((3S,4R)-3-fluoro-1-methylpiperidin-4-yl)amino)-3-(2,2,2-trifluoroethyl)benzo[b]thiophen-2-yl)-1-(pyridin-2-yl)prop-2-en-1-one

To a stirred solution of 7-(((3S,4R)-3-fluoro-1-methylpiperidin-4-yl)amino)-3-(2,2,2-trifluoroethyl)benzo[b]thiophene-2-carbaldehyde (40 mg, 0.1 mmol) in EtOH (0.3 mL) and 10% NaOH (aq, 0.1 mL) was added 1-(pyridin-2-yl) ethan-1-one (12.9 mg, 0.1 mmol) dropwise at 0° C. The resulting mixture was stirred for additional 1 h at 0° C. The reaction mixture was concentrated under reduced pressure. The residue was purified by silica gel column chromatography, eluted with DCM/MeOH (10:1) to afford (E)-3-(7-(((3S,4R)-3-fluoro-1-methylpiperidin-4-yl)amino)-3-(2,2,2-trifluoroethyl)benzo[b]thiophen-2-yl)-1-(pyridin-2-yl)prop-2-en-1-one (32 mg) as a solid. LCMS [M+H]⁺ m/z: 478.1.

### Step 2. (E)-3-(7-(((3S,4R)-3-fluoro-1-methylpiperidin-4-yl)amino)-3-(2,2,2-trifluoroethyl)benzo[b]thiophen-2-yl)-1-(pyridin-2-yl)prop-2-en-1-ol

To a stirred solution of (E)-3-(7-(((3S,4R)-3-fluoro-1-methylpiperidin-4-yl)amino)-3-(2,2,2-trifluoroethyl)benzo[b]thiophen-2-yl)-1-(pyridin-2-yl)prop-2-en-1-one (27 mg, 0.06 mmol) in MeOH (0.5 mL) was added CeCl₃ (16.7 mg, 0.07 mmol) in portions at 0° C. under air atmosphere. The resulting mixture was stirred for 10 min at 0° C. To the above mixture was added NaBH₄ (2.6 mg, 0.07 mmol) at 0° C. The resulting mixture was stirred for additional 20 min at 0° C. The resulting mixture was concentrated under reduced pressure. The residue was purified by reversed-phase flash chromatography with the following conditions: column, C18 silica gel; mobile phase, ACN in water (10 mmol/L NH₄HCO₃), 35% to 50% gradient in 10 min; detector, UV 254 nm. This resulted in (E)-3-(7-(((3S,4R)-3-fluoro-1-methylpiperidin-4-yl)amino)-3-(2,2,2-trifluoroethyl)benzo[b]thiophen-2-yl)-1-(pyridin-2-yl)prop-2-en-1-ol (7 mg) as a solid.

The compounds in the table below were synthesized as described in Example 5-1 from appropriate intermediates.

| | Chemical name | |
|---|---|---|
| Example | Chemical structure | NMR, MS |
| | (E)-3-(7-(((3R,4S)-4-fluoropyrrolidin-3-yl)amino)-3-(2,2,2- | |
| | trifluoroethyl)benzo[b]thiophen-2-yl)-1-(pyridin-2-yl)prop-2-en- | |
| | 1-ol | |
| 5-1 | 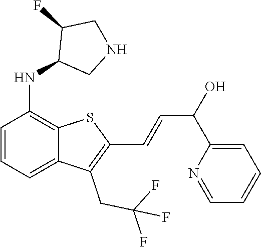 [SMILES: OC(/C=C/c1sc2c(N[C@@H]3CNC[C@@H]3F)cccc2c1CC(F)(F)F)c1ccccn1] | 1H NMR (400 MHz, Methanol-d4) δ 8.41 (m, 1H), 7.78 (m, 1H), 7.54 (dd, J = 8.0, 1.2 Hz, 1H), 7.24 (m, 1H), 7.17 (t, J = 7.9 Hz, 1H), 7.11-7.03 (m, 2H), 6.67 (d, J = 7.6 Hz, 1H), 6.36 (dd, J = 15.4, 6.1 Hz, 1H), 5.38-5.31 (m, 1H), 5.17-5.00 (m, 1H), 4.18-4.02 (m, 1H), 3.71 (m, 2H), 3.26 (dd, J = 11.2, 7.7 Hz, 2H), 3.15-3.03 (m, 1H), 2.76 (t, J = 10.1 Hz, 1H), 19F NMR (376 MHz, Methanol-d4) δ −66.00, −195.88, M + H: 452.15. |
| | (E)-3-(7-(((3S,4R)-3-fluoro-1-methylpiperidin-4-yl)amino)-3- | |
| | (2,2,2-trifluoroethyl)benzo[b]thiophen-2-yl)-1-(pyridin-2- | |
| | yl)prop-2-en-1-ol | |
| 5-2 | 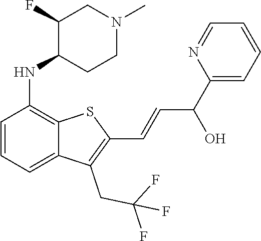 [SMILES: CN1CC[C@@H](Nc2cccc3c(CC(F)(F)F)c(/C=C/C(O)c4ccccn4)sc23)[C@@H](F)C1] | 1H NMR (400 MHz, Methanol-d4) δ 8.59- 8.46 (m, 1H), 7.88 (td, J = 7.7, 1.8 Hz, 1H), 7.64 (d, J = 7.9 Hz, 1H), 7.42-7.30 (m, 1H), 7.30-7.12 (m, 3H), 6.74 (d, J = 7.6 Hz, 1H), 6.46 (dd, J = 15.5, 6.0 Hz, 1H), 5.44 (dd, J = 6.1, 1.4 Hz, 1H), 4.77 (s, 1H), 3.91-3.60 (m, 3H), 3.20 (t, J = 11.7 Hz, 1H), 2.93 (d, J = 11.7 Hz, 1H), 2.48-2.16 (m, 5H), 2.06-1.87 (m, 2H). 19F NMR (376 MHz, Methanol-d4) δ −66.01, −203.10. M + H: 480.30 |
| | (E)-1-(3-chloro-1H-pyrazol-5-yl)-3-(7-(((3R,4S)-4- | |
| | fluoropyrrolidin-3-yl)amino)-3-(2,2,2- | |
| | trifluoroethyl)benzo[b]thiophen-2-yl)prop-2-en-1-ol | |
| 5-3 | 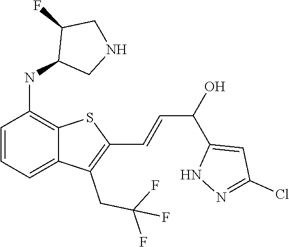 [SMILES: OC(/C=C/c1sc2c(N[C@@H]3CNC[C@@H]3F)cccc2c1CC(F)(F)F)c1cc(Cl)n[nH]1] | 1H NMR (400 MHz, Methanol-d4) δ 7.35- 7.12 (m, 3H), 6.79 (d, J = 7.6 Hz, 1H), 6.42 (dd, J = 15.4, 6.4 Hz, 1H), 6.19 (s, 1H), 5.44 (d, J = 6.5 Hz, 1H), 5.20 (dt, J = 55.2, 4.0 Hz, 1H), 4.32-4.15 (m, 1H), 3.82 (m, 2H), 3.43-3.37 (m, 1H), 3.27-3.09 (m, 2H), 2.88 (t, J = 10.3 Hz, 1H), 19F NMR (376 MHz, Methanol-d4) δ −66.00, −195.93, M + H: 475.10. |
| | (E)-3-(7-(((3S,4R)-3-fluoro-1-methylpiperidin-4-yl)amino)-3- | |
| | (2,2,2-trifluoroethyl)benzo[b]thiophen-2-yl)-1-(pyridin-3- | |
| | yl)prop-2-en-1-ol | |
| 5-4 | 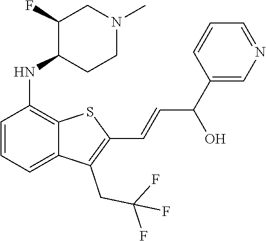 [SMILES: CN1CC[C@@H](Nc2cccc3c(CC(F)(F)F)c(/C=C/C(O)c4cccnc4)sc23)[C@@H](F)C1] | 1H NMR (400 MHz, Methanol-d4) δ 8.62 (d, J = 2.2 Hz, 1H), 8.47 (dd, J = 4.9, 1.6 Hz, 1H), 7.92 (dt, J = 8.0, 2.0 Hz, 1H), 7.46 (ddd, J = 8.0, 5.0, 0.9 Hz, 1H), 7.27 (t, J = 7.8 Hz, 1H), 7.22-7.14 (m, 2H), 6.77- 6.71 (m, 1H), 6.41 (dd, J = 15.4, 6.4 Hz, 1H), 5.48 (dd, J = 6.3, 1.3 Hz, 1H), 4.77 (s, 1H), 3.82 (q, J = 10.8 Hz, 2H), 3.76-3.64 (m, 1H), 3.20 (ddt, J = 13.0, 9.9, 3.0 Hz, 1H), 2.96-2.88 (m, 1H), 2.39 (d, J = 13.3 Hz, 1H), 2.30 (s, 3H), 2.28-2.19 (m, 1H), 1.94 (tt, J = 7.2, 3.7 Hz, 2H), 19F NMR (377 MHz, Methanol-d) δ −66.02, −202.97, M + H: 480.10. |
| | (E)-3-(7-(((3S,4R)-3-fluoro-1-methylpiperidin-4-yl)amino-3- | |
| | (2,2,2-trifluoroethyl)benzo[b]thiophen-2-yl)-1-(1-methyl-1H- | |
| | pyrazol-4-yl)prop-2-en-1-ol | |
| 5-5 | 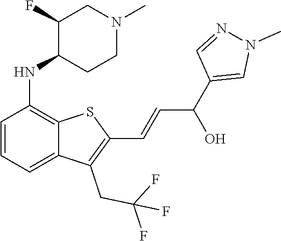 [SMILES: CN1CC[C@@H](Nc2cccc3c(CC(F)(F)F)c(/C=C/C(O)c4cnn(C)c4)sc23)[C@@H](F)C1] | 1H NMR (400 MHz, Methanol-d4) δ 8.41 (s, 1H), 7.57 (s, 1H), 7.47 (s, 1H), 7.32- 7.20 (m, 2H), 7.08 (d, J = 15.7 Hz, 1H), 6.77 (d, J = 7.5 Hz, 1H), 6.47 (dd, J = 15.4, 6.2 Hz, 1H), 5.39 (d, J = 6.4 Hz, 1H), 5.07- 5.00 (m, 1H), 3.88 (s, 3H), 3.86-3.76 (m, 3H), 3.45 (d, J = 13.0 Hz, 1H), 3.19 (d, J = 12.2 Hz, 1H), 2.79 (dd, J = 38.3, 13.5 Hz, 1H), 2.66 (d, J = 14.4 Hz, 1H), 2.55 (s, 3H), 2.08 (dd, J = 8.3, 3.5 Hz, 2H), 19F NMR (376 MHz, Methanol-d4) δ −66.02, −203.80, M + H: 483.35. |
| | (E)-3-(7-(((3S,4R)-3-fluoro-1-methylpiperidin-4-yl)amino)-3- | |
| | (2,2,2-trifluoroethyl)benzo[b]thiophen-2-yl)-1-(pyrazolo[1,5- | |
| | a]pyridin-3-yl)prop-2-en-1-ol | |
| 5-6 | 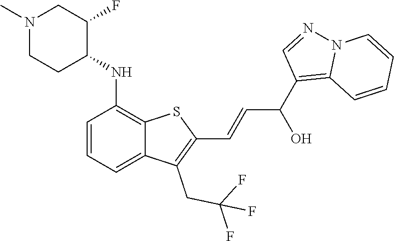 [SMILES: CN1CC[C@@H](Nc2cccc3c(CC(F)(F)F)c(/C=C/C(O)c4cnn5ccccc45)sc23)[C@@H](F)C1] | 1H NMR (400 MHz, Methanol-d4) δ 8.53 (d, J = 7.0 Hz, 1H), 7.98 (s, 1H), 7.81 (d, J = 9.0 Hz, 1H), 7.34-7.15 (m, 4H), 6.92 (t, J = 6.9 Hz, 1H), 6.77 (d, J = 7.6 Hz, 1H), 6.62 (dd, J = 15.4, 6.0 Hz, 1H), 5.76 (d, J = 5.9 Hz, 1H), 4.62 (s, 1H), 3.92-3.65 (m, 3H), 3.27-3.12 (m, 1H), 2.94 (dq, J = 12.3, 3.3 Hz, 1H), 2.46-2.18 (m, 5H), 1.96 (td, J = 10.1, 9.2, 3.9 Hz, 2H), 19F NMR (376 MHz, Methanol-d4) δ −65.97, −202.93, (M + H)+ 519.20. |
| | (E)-1-(1-(cyclopropylmethyl)-1H-pyrazol-4-yl)-3-(7-(((3S,4R)-3- | |
| | fluoro-1-methylpiperidin-4-yl)amino)-3-(2,2,2- | |
| | trifluoroethyl)benzo[b]thiophen-2-yl)prop-2-en-1-ol | |
| 5-7 | 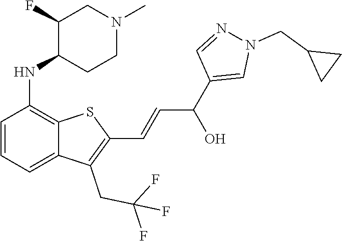 [SMILES: CN1CC[C@@H](Nc2cccc3c(CC(F)(F)F)c(/C=C/C(O)c4cnn(CC5CC5)c4)sc23)[C@@H](F)C1] | 1H NMR (400 MHz, Methanol-d4) δ 7.69 (s, 1H), 7.50 (s, 1H), 7.30-7.18 (m, 2H), 7.09 (d, J = 15.4 Hz, 1H), 6.76 (d, J = 7.6 Hz, 1H), 6.50 (dd, J = 15.4, 6.1 Hz, 1H), 5.41 (d, J = 6.2 Hz, 1H), 4.79 (s, 1H), 3.99 (d, J = 7.1 Hz, 2H), 3.86-3.68 (m, 3H), 3.22 (t, J = 11.7 Hz, 1H), 2.98-2.89 (m, 1H), 2.32 (s, 5H), 1.97 (h, J = 4.7, 4.0 Hz, 2H), 1.28 (ddt, J = 10.5, 7.4, 3.7 Hz, 1H), 0.65-0.56 (m, 2H), 0.39 (q, J = 5.2 Hz, 2H). 19F NMR (376 MHz, Methanol-d4) δ −65.98, −203.00. (M + H)+ 523.35 |
| | (E)-3-(7-(((3S,4R)-3-fluoro-1-methylpiperidin-4-yl)amino)-3- | |
| | (2,2,2-trifluoroethyl)benzo[b]thiophen-2-yl)-1-(1H-pyrazol-4- | |
| | yl)prop-2-en-1-ol | |
| 5-8 | 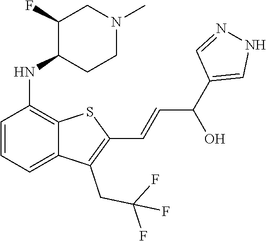 [SMILES: CN1CC[C@@H](Nc2cccc3c(CC(F)(F)F)c(/C=C/C(O)c4cn[nH]c4)sc23)[C@@H](F)C1] | 1H NMR (400 MHz, Methanol-d4) δ 7.64 (s, 2H), 7.33-7.19 (m, 2H), 7.11 (d, J = 15.4 Hz, 1H), 6.78 (d, J = 7.6 Hz, 1H), 6.53 (m, 1H), 5.47 (m, 1H), 4.92 (s, 2H), 3.83 (m, 2H), 3.31-3.17 (m, 1H), 3.05-2.85 (m, 1H), 2.34 (s, 5H), 1.99 (m, 2H). 19F NMR (376 MHz, Methanol-d4) δ −66.01, −202.97. (M + H)+ 469.15 |
| | (E)-3-(7-(((3S,4R)-3-fluoro-1-methylpiperidin-4-yl)amino)-3- | |
| | (2,2,2-trifluoroethyl)benzo[b]thiophen-2-yl)-1-(pyridin-4- | |
| | yl)prop-2-en-1-ol | |
| 5-9 | 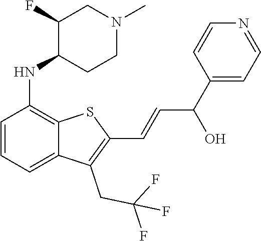 [SMILES: CN1CC[C@@H](Nc2cccc3c(CC(F)(F)F)c(/C=C/C(O)c4ccncc4)sc23)[C@@H](F)C1] | 1H NMR (400 MHz, Methanol-d4) δ 8.59- 8.47 (m, 2H), 7.59-7.47 (m, 2H), 7.31- 7.12 (m, 3H), 6.75 (d, J = 7.6 Hz, 1H), 6.35 (dd, J = 15.4, 6.6 Hz, 1H), 5.43 (d, J = 6.6 Hz, 1H), 4.78 (m, 1H), 3.83 (m, 2H), 3.69 (m, 1H), 3.21 (m, 1H), 2.93 (d, J = 11.8 Hz, 1H), 2.45-2.21 (m, 5H), 1.95 (m, 2H). 19F NMR (376 MHz, Methanol-d4) δ −66.01, −203.04. (M + H)+ 480.10 |
| | 6-((E)-3-(7-(((3R,4S)-4-fluoropyrrolidin-3-yl)amino)-3-(2,2,2- | |
| | trifluoroethyl)benzo[b]thiophen-2-yl)-1-hydroxyallyl)pyridin- | |
| | 2(1H)-one | |
| 5-10 | 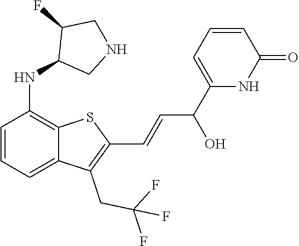 [SMILES: O=c1cccc(C(O)/C=C/c2sc3c(N[C@@H]4CNC[C@@H]4F)cccc3c2CC(F)(F)F)[nH]1] | 1H NMR (400 MHz, Methanol-d4) δ 8.50 (s, 1H), 7.54-7.45 (m, 1H), 7.24-7.06 (m, 3H), 6.71 (d, J = 7.5 Hz, 1H), 6.39- 6.20 (m, 3H), 5.30-5.07 (m, 2H), 4.36- 4.20 (m, 1H), 3.83-3.68 (m, 2H), 3.51- 3.29 (m, 3H), 3.06-2.97 (m, 1H). 19F NMR (377 MHz, Methanol-d4) δ −66.08, −197.06. (M + H)+ 468.20 |

### Example 6-1

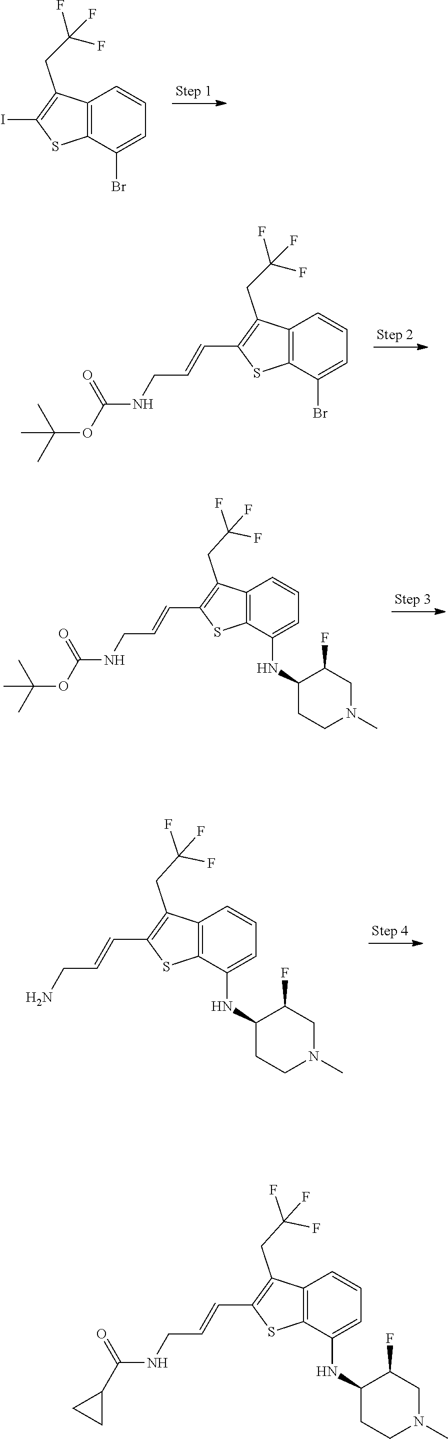 [SMILES: FC(F)(F)Cc1c(I)sc2c(Br)cccc12 ; CC(C)(C)OC(=O)NC/C=C/c1sc2c(Br)cccc2c1CC(F)(F)F ; CN1CC[C@@H](Nc2cccc3c(CC(F)(F)F)c(/C=C/CNC(=O)OC(C)(C)C)sc23)[C@@H](F)C1 ; CN1CC[C@@H](Nc2cccc3c(CC(F)(F)F)c(/C=C/CN)sc23)[C@@H](F)C1 ; CN1CC[C@@H](Nc2cccc3c(CC(F)(F)F)c(/C=C/CNC(=O)C4CC4)sc23)[C@@H](F)C1]

### Step 1. tert-butyl (E)-(3-(7-bromo-3-(2,2,2-trifluoroethyl)benzo[b]thiophen-2-yl)allyl)carbamate

To a stirred mixture of 7-bromo-2-iodo-3-(2,2,2-trifluoroethyl)-1-benzothiophene (200 mg, 0.475 mmol), K₂CO₃ (131.31 mg, 0.950 mmol), RuPhos Palladacycle Gen.3 (39.73 mg, 0.048 mmol) in dioxane (5 mL) was added t-butyl N-[(2E)-3-(4,4,5,5-tetramethyl-1,3,2-dioxaborolan-2-yl)prop-2-en-1-yl]carbamate (134.5 mg, 0.475 mmol) in portions at rt under nitrogen atmosphere. The resulting mixture was stirred for 24 h at 100° C. under nitrogen atmosphere. The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. to afford tert-butyl (E)-(3-(7-bromo-3-(2,2,2-trifluoroethyl)benzo[b]thiophen-2-yl)allyl)carbamate.

### Step 2. tert-butyl ((E)-3-(7-(((3S,4R)-3-fluoro-1-methylpiperidin-4-yl)amino)-3-(2,2,2-trifluoroethyl)benzo[b]thiophen-2-yl)allyl)carbamate

To a stirred mixture of tert-butyl N-[(2E)-3-(7-{[(3S,4R)-3-fluoro-1-methylpiperidin-4-yl]amino}-3-(2,2,2-trifluoroethyl)-1-benzothiophen-2-yl)prop-2-en-1-yl]carbamate (160 mg, 0.319 mmol) in DCM (4 mL, 62.9 mmol) was added TFA (1 mL, 13.5 mmol) in portions at 0° C. The resulting mixture was stirred for 1 h at rt. The reaction was monitored by LCMS. Desired product could be detected by LCMS. The resulting mixture was concentrated under reduced pressure. The mixture was basified to pH 8 with saturated NaHCO₃ (aq.). The aqueous layer was extracted with EtOAc (3×10 mL). The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. to afford (3S,4R)—N-{2-[(1E)-3-aminoprop-1-en-1-yl]-3-(2,2,2-trifluoroethyl)-1-benzothiophen-7-yl}-3-fluoro-1-methylpiperidin-4-amine.

### Step 3. (3S,4R)—N-(2-((E)-3-aminoprop-1-en-1-yl)-3-(2,2,2-trifluoroethyl)benzo[b]thiophen-7-yl)-3-fluoro-1-methylpiperidin-4-amine

To a stirred mixture of tert-butyl N-[(2E)-3-(7-{[(3S,4R)-3-fluoro-1-methylpiperidin-4-yl]amino}-3-(2,2,2-trifluoroethyl)-1-benzothiophen-2-yl)prop-2-en-1-yl]carbamate (160 mg, 0.319 mmol) in DCM (4 mL, 62.922 mmol) was added TFA (1 mL, 13.463 mmol) in portions at 0° C. The resulting mixture was stirred for 1 h at rt. The reaction was monitored by LCMS. Desired product could be detected by LCMS. The resulting mixture was concentrated under reduced pressure. The mixture was basified to pH 8 with saturated NaHCO₃ (aq.). The aqueous layer was extracted with EtOAc (3×10 mL). The resulting mixture was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 254 nm. to afford (3S,4R)—N-{2-[(1E)-3-aminoprop-1-en-1-yl]-3-(2,2,2-trifluoroethyl)-1-benzothiophen-7-yl}-3-fluoro-1-methylpiperidin-4-amine.

### Step 4. N—((E)-3-(7-(((3S,4R)-3-fluoro-1-methylpiperidin-4-yl)amino)-3-(2,2,2-trifluoroethyl)benzo[b]thiophen-2-yl)allyl)cyclopropanecarboxamide

A solution of cyclopropanecarboxylic acid (5.16 mg, 0.060 mmol) in DMF (2 mL) was treated with EDCI (14.36 mg, 0.075 mmol), HOBt (10.12 mg, 0.075 mmol) and DIEA (19.36 mg, 0.150 mmol) for 0.5 h at rt under nitrogen atmosphere followed by the addition of (3S,4R)—N-{2-[(1E)-but-1-en-1-yl]-3-(2,2,2-trifluoroethyl)-1-benzothiophen-7-yl}-3-fluoro-1-methylpiperidin-4-amine (20 mg, 0.050 mmol) at rt. The resulting mixture was stirred for 2 h at rt under nitrogen atmosphere. The reaction was monitored by LCMS. Desired product could be detected by LCMS. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. to afford N-[(2E)-3-(7-{[(3S,4R)-3-fluoro-1-methylpiperidin-4-yl]amino}-3-(2,2,2-trifluoroethyl)-1-benzothiophen-2-yl)prop-2-en-1-yl]cyclopropanecarboxamide.

The compounds in the table below were synthesized as described in Example 6-1 from appropriate intermediates.

| | Chemical name | |
|---|---|---|
| Example | Chemical structure | NMR, MS |
| | N-((E)-3-(7-(((3S,4R)-3-fluoro-1-methylpiperidin-4-yl)amino)-3- | |
| | (2,2,2-trifluoroethyl)benzo[b]thiophen-2- | |
| | yl)allyl)cyclopropanecarboxamide | |
| 6-1 | 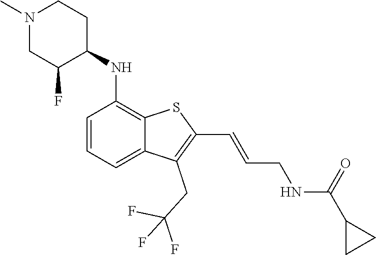 [SMILES: CN1CC[C@@H](Nc2cccc3c(CC(F)(F)F)c(/C=C/CNC(=O)C4CC4)sc23)[C@@H](F)C1] | 1H NMR (400 MHz, Chloroform-d) δ 7.28 (d, J = 7.8 Hz, 1H), 7.15 (d, J = 8.0 Hz, 1H), 6.80 (d, J = 15.5 Hz, 1H), 6.62 (d, J = 7.7 Hz, 1H), 6.34-6.14 (m, 1H), 5.78 (s, 1H), 4.86 (d, J = 48.8 Hz, 1H), 4.20-4.05 (m, 2H), 3.86 (d, J = 9.7 Hz, 1H), 3.68-3.58 (m, 3H), 3.35-3.13 (m, 1H), 2.96 (d, J = 11.6 Hz, 1H), 2.37 (s, 3H), 2.21 (s, 2H), 2.08-1.91 (m, 2H), 1.43-1.33 (m, 1H), 1.09-0.93 (m, 2H), 0.87-0.69 (m, 2H), 19F NMR (400 MHz, Chloroform-d) δ −64.454, −201.583, (M + H)+ 470.20. |
| | N-((E)-3-(7-(((3S,4R)-3-fluoro-1-methylpiperidin-4-yl)amino)-3- | |
| | (2,2,2-trifluoroethyl)benzo[b]thiophen-2-yl)allyl)-1-methyl-1H- | |
| | pyrazole-4-carboxamide | |
| 6-2 | 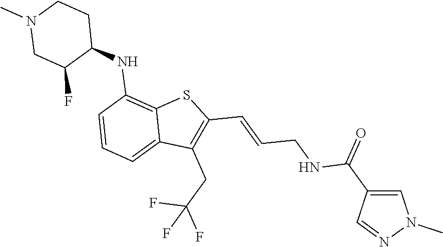 | 1H NMR (400 MHz, Chloroform-d) δ 7.86 (s, 1H), 7.76 (s, 1H), 7.28 (d, J = 7.9 Hz, 1H), 7.15 (d, J = 8.0 Hz, 1H), 6.85 (d, J = 15.5 Hz, 1H), 6.62 (d, J = 7.7 Hz, 1H), 6.42-6.23 (m, 1H), 5.95 (s, 1H), 4.87 (d, J = 48.6 Hz, 1H), 4.34-4.14 (m, 2H), 3.93 (s, 3H), 3.85 (d, J = 9.6 Hz, 1H), 3.71- 3.54 (m, 3H), 3.25 (d, J = 11.7 Hz, 1H), 2.98 (d, J = 11.6 Hz, 1H), 2.40 (s, 4H), 2.25 (s, 1H), 2.01 (d, J = 14.8 Hz, 2H), 19F NMR (400 MHz, Chloroform-d) δ −64.431, −201.560, (M + H)+ 510.40. |
| | N-((E)-3-(7-(((3S,4R)-3-fluoro-1-methylpiperidin-4-yl)amino)-3- | |
| | (2,2,2-trifluoroethyl)benzo[b]thiophen-2- | |
| | yl)allyl)methanesulfonamide | |
| 6-3 | 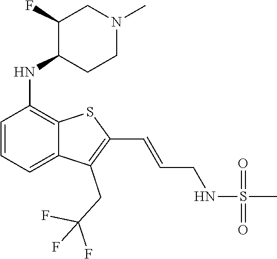 [SMILES: CN1CC[C@@H](Nc2cccc3c(CC(F)(F)F)c(/C=C/CNS(C)(=O)=O)sc23)[C@@H](F)C1] | 1H NMR (400 MHz, Chloroform-d) δ 7.29 (d, J = 7.9 Hz, 1H), 7.16 (d, J = 8.0 Hz, 1H), 6.91 (d, J = 15.4 Hz, 1H), 6.63 (d, J = 7.7 Hz, 1H), 6.34-6.20 (m, 1H), 4.97-4.76 (m, 2H), 4.05-3.95 (m, 3H), 3.73-3.55 (m, 3H), 3.31-3.20 (m, 1H), 3.00 (s, 4H), 2.37 (s, 5H), 2.08-1.86 (m, 2H), 19F NMR (400 MHz, Chloroform- d) δ −64.435, −201.363, (M + H)+ 480.35. |
| | methyl ((E)-3-(7-(((3S,4R)-3-fluoro-1-methylpiperidin-4 | |
| | yl)amino)-3-(2,2,2-trifluoroethyl)benzo[b]thiophen-2- | |
| | yl)allyl)carbamate | |
| 6-4 | 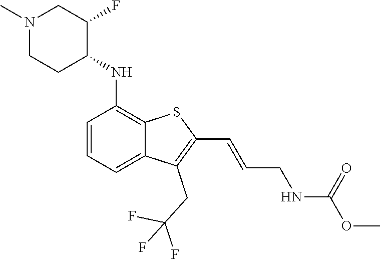 | 1H NMR (400 MHz, Methanol-d4) δ 7.26 (m, 1H), 7.18 (d, J = 8.0 Hz, 1H), 6.93 (d, J = 15.5 Hz, 1H), 6.75 (d, J = 7.6 Hz, 1H), 6.27 (m, 1H), 4.78 (s, 1H), 3.92 (m, 2H), 3.79 (m, 3H), 3.68 (s, 3H), 3.21 (m, 1H), 2.93 (m, 1H), 2.40 (m, 1H), 2.31 (s, 3H), 2.27-2.19 (m, 1H), 1.96 (m, 2H), 19F NMR (377 MHz, MeOD) δ −66.03, −203.03, (M + H)+ 460.10. |

### Example 7-1

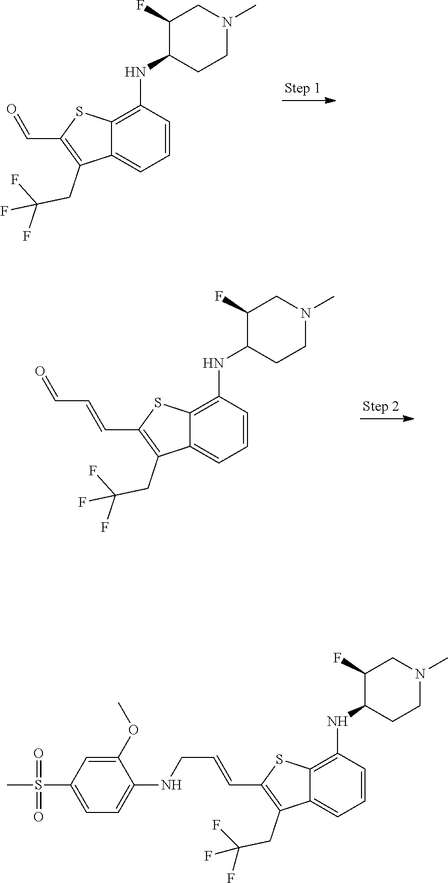

### Step 1. (E)-3-(7-(((3S,4R)-3-fluoro-1-methylpiperidin-4-yl)amino)-3-(2,2,2-trifluoroethyl)benzo[b]thiophen-2-yl) acrylaldehyde

To a stirred solution of 7-(((3S,4R)-3-fluoro-1-methylpiperidin-4-yl)amino)-3-(2,2,2-trifluoroethyl)benzo[b]thiophene-2-carbaldehyde (400 mg, 1.1 mmol) and ((1,3-dioxolan-2-yl)methyl)triphenylphosphonium bromide (688 mg, 1.6 mmol) in DCM (4 mL) were added tris(2-(2-methoxyethoxy)ethyl)amine (363 mg, 1.1 mmol) and sat. K₂CO₃ (aq., 2 mL) at 0° C. under nitrogen atmosphere. The resulting mixture was stirred for 2 h at 60° C. under nitrogen atmosphere. The reaction mixture was diluted with water (10 mL) at rt. The resulting mixture was extracted with DCM (3×10 mL). The combined organic layers were concentrated under reduced pressure. To the above residue was added THF (2 mL), AcOH (2 mL) and H₂O (2 mL) at rt. The resulting mixture was stirred for 4 h at 60° C. The resulting mixture was concentrated under reduced pressure. The residue was purified by silica gel column chromatography, eluted with MeOH in EA (0%-10%) to afford (E)-3-(7-(((3S,4R)-3-fluoro-1-methylpiperidin-4-yl)amino)-3-(2,2,2-trifluoroethyl)benzo[b]thiophen-2-yl) acrylaldehyde (300 mg) as a solid. LCMS [M+H]⁺ m/z: 401.3.

### Step 2. (3S,4R)-3-fluoro-N-(2-((E)-3-((2-methoxy-4-(methylsulfonyl)phenyl)amino)prop-1-en-1-yl)-3-(2,2,2-trifluoroethyl)benzo[b]thiophen-7-yl)-1-methylpiperidin-4-amine

A solution of (E)-3-(7-(((3S,4R)-3-fluoro-1-methylpiperidin-4-yl)amino)-3-(2,2,2-trifluoroethyl)benzo[b]thiophen-2-yl) acrylaldehyde (20 mg, 0.05 mmol) and 2-methoxy-4-(methylsulfonyl) aniline (12.1 mg, 0.06 mmol) in MeOH (1 mL) was added AcOH (9 mg, 0.2 mmol) at rt. The resulting mixture was stirred for 10 min. To the above mixture was added NaBH₃CN (3.8 mg, 0.06 mmol) at 0° C. The resulting mixture was stirred for additional 6 h at rt. The reaction mixture was concentrated under reduced pressure. The residue was purified by reversed-phase flash chromatography with the following conditions: column, C18 silica gel; mobile phase, ACN in water (0.1% NH₃·H₂O+10 mmol/L NH₄HCO₃), 30% to 50% gradient in 20 min; detector, UV 254. This resulted in (3S,4R)-3-fluoro-N-(2-((E)-3-((2-methoxy-4-(methylsulfonyl)phenyl)amino)prop-1-en-1-yl)-3-(2,2,2 trifluoroethyl)benzo[b]thiophen-7-yl)-1-methylpiperidin-4-amine (6 mg) as a solid.

The compounds in the table below were synthesized as described in Example 7-1 from appropriate intermediates.

| | Chemical name | |
|---|---|---|
| Example | Chemical structure | NMR, MS |
| | N-((E)-3-(7-(((3S,4R)-3-fluoro-1-methylpiperidin-4-yl)amino)-3- | |
| | (2,2,2-trifluoroethyl)benzo[b]thiophen-2-yl)allyl)pyridin-3- | |
| | amine | |
| 7-1 | 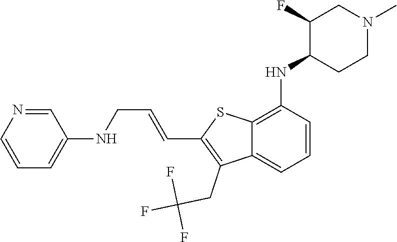 [SMILES: CN1CC[C@@H](Nc2cccc3c(CC(F)(F)F)c(/C=C/CNc4cccnc4)sc23)[C@@H](F)C1] | 1H NMR (400 MHz, Methanol-d4) δ 7.98 (dd, J = 2.9, 0.8 Hz, 1H), 7.78 (dd, J = 4.7, 1.4 Hz, 1H), 7.25 (t, J = 7.8 Hz, 1H), 7.20-7.12 (m, 2H), 7.09 (m, 1H), 7.03 (dt, J = 15.5, 1.8 Hz, 1H), 6.74 (d, J = 7.6 Hz, 1H), 6.36 (dt, J = 15.5, 5.3 Hz, 1H), 4.90 (s, 1H), 4.02 (dd, J = 5.3, 1.7 Hz, 2H), 3.82-3.65 (m, 3H), 3.27-3.16 (m, 1H), 2.93 (dd, J = 11.4, 2.6 Hz, 1H), 2.45-2.18 (m, 5H), 1.95 (m, 2H), 19F NMR (376 MHz, Methanol-d4) δ −66.10, −203.01, M + H: 479.20. |
| | (3S,4R)-3-fluoro-N-(2-((E)-3-((2-methoxy-4- | |
| | (methylsulfonyl)phenyl)amino)prop-1-en-1-yl)-3-(2,2,2- | |
| | trifluoroethyl)benzo[b]thiophen-7-yl)-1-methylpiperidin-4- | |
| | amine | |
| 7-2 | 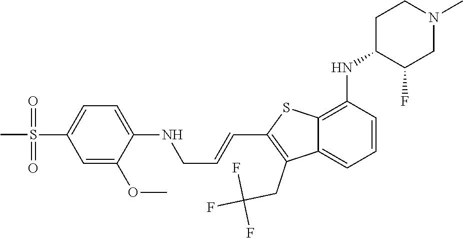 | 1H NMR (400 MHz, Methanol-d4) δ 7.40 (m, 1H), 7.31-7.21 (m, 2H), 7.17 (d, J = 8.0 Hz, 1H), 6.98 (d, J = 15.6 Hz, 1H), 6.81-6.64 (m, 2H), 6.33 (m, 1H), 4.91- 4.83 (m, 1H), 4.12 (dt, J = 5.5, 1.6 Hz, 2H), 3.96 (d, J = 1.0 Hz, 3H), 3.81-3.63 (m, 3H), 3.20 (t, J = 11.7 Hz, 1H), 3.04 (d, J = 0.7 Hz, 3H), 2.93 (d, J = 11.8 Hz, 1H), 2.45-2.17 (m, 5H), 1.96 (dd, J = 8.5, 3.5 Hz, 2H), 19F NMR (377 MHz, Methanol-d4) δ −66.05, −203.05, M + H: 586.20. |

Biological Assays to Measure the Activities of p53-Y220C Reactivators

### Biological Example 1. Biochemical p53-Y220C and DNA Binding Assay

The compounds of this invention can bind to p53-Y220C and increase the ability of the mutant p53 to bind to DNA at higher temperatures. His-tagged p53-Y220C DNA binding domain containing amino acid 94-312 is used to measure DNA binding activities in vitro with the sequence described below. (SEQ ID NO: 1: MHHHHHHENLYFQGSSSVPSQKTYQGSYGFRLGFLHSGTAKSVTCTYSPALNKMFCQL AKTCPVQLWVDSTPPPGTRVRAMAIYKQSQHMTEVVRRCPHHERCSDSDGLAPPQHLI RVEGNLRVEYLDDRNTFRHSVVVPCEPPEVGSDCTTIHYNYMCNSSCMGGMNRRPILTI ITLEDSSGNLLGRNSFEVRVCACPGRDRRTEEENLRKKGEPHHELPPGSTKRALPNNT. Biotin labeled double strand DNA (dsDNA) containing consensus p53 binding sequence (SEQ ID NO: 2: Forward: 5′-(biotin)-ATTAGGCATGTCTAGGCATGTCTAGG-3′; Reverse: 5′-(biotin)-CCTAGACATGCCTAGACATGCCTAAT-3′) is used to measure protein-DNA binding activities. Compounds, His-tagged p53-Y220C DBD proteins (100 nM), and biotinylated dsDNA (200 nM) were mixed in ice-cold assay buffer containing DPBS, 20 mM NaCl, and 0.5% BAS, and incubated at 4° C. overnight in 384-well plate. Plates were transferred to incubator at 27 to 29° C. with constant shaking for 60 minutes. Equal volume of Homogeneous Time-Resolved Fluorescence (HTRF) dyes containing mAb anti-6HIS Tb cryptate gold and d2 labeled streptavidin in the assay buffer was added to each well and incubated at 27 to 29° C. with constant shaking for 60 minutes. The plate was read using Envision multimode plate reader. Reference compound 1 was used as high control and DMSO was used as low control. The percentage activation (A %) of protein-DNA binding by compounds was normalized by setting up high control (reference compound) as 500% and low control (DMSO) as 0% (A %=(HTRF ratio of compound-HTRF ratio of low control)/(HTRF ratio of high control-HTRF ratio of low control)*500). 10 points dose titration curves for each compound were analyzed by 4 parameter curve fit and inflection point (IP) was reported. Reference compound 1 is shown below Table 1. Compounds of the invention are active in the HTRF assay. Data in Table 1 collected using Biological Example 1.

TABLE 1

| | HTRF IP |
|---|---|
| Example | (nM) |
| 1-1 | B |
| 1-2 | C |
| 1-3 | B |
| 1-4 | C |
| 1-5 | B |
| 1-6 | B |
| 1-7 | A |
| 1-8 | B |
| 1-9 | C |
| 1-10 | B |
| 1-11 | C |
| 1-12 | B |
| 1-13 | C |
| 1-14 | B |
| 1-15 | B |
| 1-16 | C |
| 1-17 | B |
| 1-18 | B |
| 1-19 | B |
| 1-20 | B |
| 1-21 | C |
| 1-22 | B |
| 1-23 | C |
| 1-24 | B |
| 1-25 | C |
| 1-26 | C |
| 1-27 | B |
| 1-28 | C |
| 1-29 | C |
| 1-30 | C |
| 1-31 | C |
| 1-32 | C |
| 1-33 | C |
| 1-34 | C |
| 1-35 | B |
| 1-36 | B |
| 1-37 | A |
| 1-38 | B |
| 1-39 | A |
| 1-40 | B |
| 2-1 | C |
| 2-2 | C |
| 2-3 | C |
| 2-4 | C |
| 2-5 | C |
| 2-6 | C |
| 2-7 | C |
| 2-8 | B |
| 2-9 | B |
| 2-10 | B |
| 2-11 | B |
| 2-12 | B |
| 2-13 | B |
| 2-14 | C |
| 2-15 | B |
| 2-16 | C |
| 2-17 | B |
| 2-18 | B |
| 2-19 | B |
| 2-20 | B |
| 2-21 | B |
| 2-22 | A |
| 2-23 | B |
| 2-24 | B |
| 2-25 | B |
| 2-26 | A |
| 2-27 | B |
| 2-28 | B |
| 2-29 | B |
| 2-30 | B |
| 2-31 | A |
| 2-32 | B |
| 2-33 | A |
| 2-34 | C |
| 3-1 | A |
| 3-2 | A |
| 3-3 | B |
| 3-4 | B |
| 3-5 | B |
| 3-6 | B |
| 3-7 | B |
| 3-8 | B |
| 4-1 | C |
| 4-2 | C |
| 5-1 | C |
| 5-2 | C |
| 5-3 | B |
| 5-4 | B |
| 5-5 | B |
| 5-6 | B |
| 5-7 | B |
| 5-8 | B |
| 5-9 | B |
| 5-10 | B |
| 6-1 | B |
| 6-2 | B |
| 6-3 | B |
| 6-4 | B |
| 7-1 | B |
| 7-2 | A |

A < 100 nM, B = 100-1000 nM, C > 1000 nM

### Reference Compound 1 (HTRF=A; IP<100 nM)

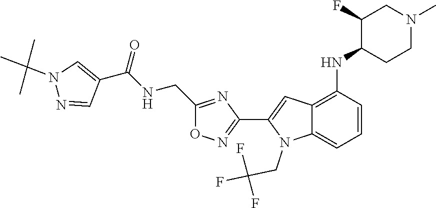 [SMILES: CN1CC[C@@H](Nc2cccc3c2cc(-c2noc(CNC(=O)c4cnn(C(C)(C)C)c4)n2)n3CC(F)(F)F)[C@@H](F)C1]

What is claimed is:

1. A compound represented by the following structural formula: 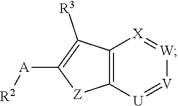
or a pharmaceutically acceptable salt thereof, wherein:
A is represented by 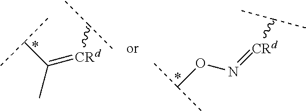
wherein “*” indicates the point of attachment to R²;
Z is S, O, or NR^{3a};
U, V, W and X are independently CR⁵ or N, provided that one of U, V, W and X is C—Y—R¹;
Y is O, NH, N(C₁₋₄alkyl), NHCH₂\hat{ }, OCH₂\hat{ }, S or CH₂, wherein “\hat{ }” indicates the point of attachment to R¹;
R¹ is (CH₂)ₙOR¹¹, (CH₂)ₙN(R¹¹)₂, (CH₂)ₙCN, (CH₂)ₙC(O)R¹¹, (CH₂)ₙC(O)OR¹¹, (CH₂)ₙC(S)R¹¹, (CH₂)ₙC(S)OR¹¹, (CH₂)ₙC(O)N(R¹¹)₂, (CH₂)ₙNHC(O)R¹¹, (CH₂)ₙNHC(O)OR¹¹, (CH₂)ₙOC(O)N(R¹¹)₂, (CH₂)ₙC(S)N(R¹¹)₂, (CH₂)ₙNHC(S)R¹¹, (CH₂)ₙNHC(S)OR¹¹, (CH₂)ₙOC(S)N(R¹¹)₂, (CH₂)ₙNHS(O)ᵢR¹¹, (CH₂) \hat{ }S(O)ᵢN(R¹¹)₂, C₁₋₆ alkyl, C₃₋₈ cycloalkyl, C₂₋₆ alkenyl, C₂₋₆ alkynyl, C₆₋₁₀ aryl, 5-10 membered heteroaryl, or 4-10 membered heterocyclyl, wherein said alkyl, alkenyl, alkynyl, cycloalkyl, aryl, heteroaryl and heterocyclyl are optionally and independently substituted by one or more R²¹;
R² is independently (CH₂)ₘOR¹², (CH₂)ₘN(R¹²)₂, (CH₂)ₘCN, (CH₂)ₘC(O)R¹², (CH₂)ₘC(O)OR¹², (CH₂)ₘC(S)R¹², (CH₂)ₘC(S)OR¹², (CH₂)ₘC(O)N(R¹²)₂, (CH₂)ₘNHC(O)R¹², (CH₂)ₘNHC(O)OR¹², (CH₂)ₘOC(O)N(R¹²)₂, (CH₂)ₘC(S)N(R¹²)₂, (CH₂)ₘNHC(S)R¹², (CH₂)ₘNHC(S)OR¹², (CH₂)ₘOC(S)N(R¹²)₂, (CH₂)ₘNHS(O)ᵢR¹², (CH₂)ₘS(O)ᵢN(R¹²)₂, C₁₋₆ alkyl, (CH₂)ₘC₃₋₈ cycloalkyl, C₂₋₆ alkenyl, C₂₋₆ alkynyl, (CH₂)ₘC₆₋₁₀ aryl, 5-10 membered heteroaryl or (CH₂)ₘ(4-10 membered heterocyclyl), wherein:
said alkyl, alkenyl and alkynyl are each optionally and independently substituted by one or more R^{22b};
said aryl, and heteroaryl are each optionally and independently substituted by one or more R²²; and
said cycloalkyl and heterocyclyl are each optionally substituted by one or more R^{22a};
when Z is O or S, R³ is H, halo, (CH₂)ₒOR¹³, (CH₂)ₒSR¹³, (CH₂)ₒN(R¹³)₂, (CH₂)ₒCN, (CH₂)ₒC(O)R¹³, CH₂)ₒC(O)OR¹³, (CH₂)ₒC(S)R¹³, (CH₂)ₒC(S)OR¹³, (CH₂)ₒC(S)OR¹³ (CH₂)ₒC(O)N(R¹³)₂, (CH₂)ₒNHC(O)R¹³, (CH₂)ₒNHC(O)OR¹³, (CH₂)ₒOC(O)N(R¹³)₂, (CH₂)ₒC(S)N(R¹³)₂, (CH₂)ₒNHC(S)R¹³, (CH₂)ₒNHC(S)OR¹³, (CH₂)ₒOC(S)N(R¹³)₂, (CH₂)ₒNHS(O)ᵢR¹³, (CH₂)ₒS(O)ᵢN(R¹³)₂, C₁₋₆ alkyl, C₃₋₈ cycloalkyl, C₂₋₆ alkenyl or C₂₋₆ alkynyl, wherein said alkyl, alkenyl, alkynyl and cycloalkyl are each optionally and independently substituted by one or more R²³;
when Z is NR^{3a}, R³ is H, halo, (CH₂)ₒOR¹³, (CH₂)ₒSR¹³, C₁₋₆ alkyl or C₃₋₆ cycloalkyl, wherein said alkyl and cycloalkyl are each optionally and independently substituted by one or more R²³;
R^{3a} is H, (CH₂)ₒOR¹³, (CH₂)ₒSR¹³, (CH₂)ₒN(R¹³)₂, (CH₂)ₒCN, (CH₂)ₒC(O)R¹³, CH₂)ₒC(O)OR¹³, (CH₂)ₒC(S)R¹³, (CH₂)ₒC(S)OR¹³, (CH₂)ₒC(S)OR¹³ (CH₂)ₒC(O)N(R¹³)₂, (CH₂)ₒNHC(O)R¹³, (CH₂)ₒNHC(O)OR¹³, (CH₂)ₒOC(O)N(R¹³)₂, (CH₂)ₒC(S)N(R¹³)₂, (CH₂)ₒNHC(S)R¹³, (CH₂)ₒNHC(S)OR¹³, (CH₂)ₒOC(S)N(R¹³)₂, (CH₂)ₒNHS(O)ᵢR¹³, (CH₂)ₒS(O)ᵢN(R¹³)₂, C₁₋₆ alkyl, C₃₋₈ cycloalkyl, C₂₋₆ alkenyl or C₂₋₆ alkynyl, wherein said alkyl, alkenyl, alkynyl and cycloalkyl are each optionally and independently substituted by one or more R²³;
R⁴ is H, halo, CN, C₁₋₆ alkyl, or C₃₋₈ cycloalkyl, wherein said alkyl, and cycloalkyl are each optionally and independently substituted by one or more R²⁴; or
R² and R⁴ are optionally taken together with their intervening atom to form a C₃₋₁₀ non-aromatic carbocyclyl, or 4-10 membered heterocyclyl, wherein said carbocyclyl or heterocyclyl are each optionally and independently substituted by one or more R²²; and
each R⁵ is independently H, halo, (CH₂)ₚOR¹⁵, (CH₂)ₚN(R¹⁵)₂, (CH₂)ₚCN, (CH₂)ₒC(O)R¹⁵, (CH₂)ₚC(O)OR¹⁵, (CH₂)ₚC(S)R¹⁵, (CH₂)ₚC(S)OR¹⁵, (CH₂)ₚC(O)N(R¹⁵)₂, (CH₂)ₚNHC(O)R¹⁵, (CH₂)ₚNHC(O)OR¹⁵, (CH₂)ₚOC(O)N(R¹⁵)₂, (CH₂)ₚC(S)N(R¹⁵)₂, (CH₂)ₚNHC(S)R¹⁵, (CH₂)ₚNHC(S)OR¹⁵, (CH₂)ₚOC(S)N(R¹⁵)₂, (CH₂)ₙNHS(O)ᵢR¹⁵, (CH₂)ₚS(O)ᵢN(R¹⁵)₂, C₁₋₆ alkyl, C₃₋₈ cycloalkyl, C₂₋₆ alkenyl, C₂₋₆ alkynyl, C₆₋₁₀ aryl, 5-10 membered heteroaryl or 4-10 membered heterocyclyl, wherein said alkyl, alkenyl, alkoxy, alkynyl, cycloalkyl, aryl, heteroaryl or heterocyclyl is optionally and independently substituted by one or more R²⁵;
R¹¹, R¹², R¹³, and R¹⁵ are each independently H, C₁₋₆ alkyl, C₃₋₈ cycloalkyl, (CH₂) C₃₋₈ cycloalkyl, C₆₋₁₀ aryl, 5-10 membered heteroaryl or 4-10 membered heterocyclyl, wherein said alkyl, cycloalkyl, aryl, heteroaryl and heterocyclyl are optionally and independently substituted by one or more R³⁰; or N(R¹¹)₂ and N(R¹²)₂ are independently a 4-10 membered heterocyclyl, wherein the heterocyclyl represented by N(R¹¹) is optionally substituted with one or more R²¹ and the heterocyclyl represented by N(R¹²) is optionally substituted with one or more R²²;
R²¹, R²², R²³, R²⁴ and R²⁵ are each independently halo, CN, NO₂, (CH₂)ₙOR^{a}, (CH₂)ₙNR^{b}R^{c}, S(O)ᵢR^{b}, (═NH)NR^{b}R^{c}, NHS(O)ᵢR^{c}, S(O)ᵢNR^{b}R^{c}, C(═O)OR^{b}, OC(═O)OR^{b}, C(═S)OR^{b}, O(C═S)R^{b}, C(═O)NR^{b}R^{c}, NR^{b}C(═O)R^{c}, C(═S)NR^{b}R^{c}, NHC(═S)R^{c}, NH(C═O)OR^{c}, O(C═O)NR^{b}R^{c}, NH(C═S)OR^{c}, O(C═S)NR^{b}R^{c}, NH(C═O)NR^{b}R^{c}, NH(C═S)NR^{b}R^{c}, C(═S)R^{b}, C(═O)R^{b}, halo(C₁-C₅)alkyl, (C₁-C₅)alkyl, (C₂-C₅)alkenyl or (C₂-C₅)alkynyl;
each R^{22a} is independently halo, CN, NO₂, (CH₂)ₙOR^{a}, (CH₂)ₙNR^{b}R^{c}, S(O)ᵢR^{b}, (═NH)NR^{b}R^{c}, NHS(O)ᵢR^{c}, S(O)ᵢNR^{b}R^{c}, C(═O)OR^{b}, C(═S)OR^{b}, O(C═S)R^{b}, C(═O)NR^{b}R^{c}, NHC(═O)R^{c}, C(═S)NR^{b}R^{c}, NHC(═S)R^{c}, NH(C═O)OR^{c}, O(C═O)NR^{b}R^{c}, NH(C═S)OR^{c}, O(C═S)NR^{b}R^{c}, NH(C═O)NR^{b}R^{c}, NH(C═S)NR^{b}R^{c}, C(═S)R^{b}, C(═O)R^{b}, halo(C₁-C₅)alkyl, (C₁-C₅)alkyl, (C₂-C₅)alkenyl or (C₂-C₅)alkynyl, or two R^{22a} groups on the same ring atom taken together with their intervening atom are a (C₃₋₆)spirocycloalkyl or oxo, or two R^{22a} on adjacent ring atoms, taken together their intervening atoms form a phenyl, or a 5-6 membered heteroaryl, wherein said phenyl or heteroaryl is optionally substituted with one or more groups selected from halo, methyl, halomethyl, methoxy or halomethoxy;
each R^{22b} is independently halo, CN, NO₂, (CH₂)ₙOR^{a}, (CH₂)ₙNR^{b}R^{c}, S(O)ᵢR^{b}, (═NH)NR^{b}R^{c}, NHS(O)ᵢR^{c}, S(O)ᵢNR^{b}R^{c}, C(═O)OR^{b}, OC(═O)OR^{b}, C(═S)OR^{b}, O(C═S)R^{b}, C(═O)NR^{b}R^{c}, NHC(═O)R^{c}, C(═S)NR^{b}R^{c}, NHC(═S)R^{c}, NH(C═O)OR^{c}, O(C═O)NR^{b}R^{c}, NH(C═S)OR^{c}, O(C═S)NR^{b}R^{c}, NH(C═O)NR^{b}R^{c}, NH(C═S)NR^{b}R^{c}, C(═S)R^{b}, C(═O)R^{b}, halo(C₁-C₅)alkyl, (C₁-C₅)alkyl, (C₂-C₅)alkenyl, (C₂-C₅)alkynyl, phenyl or 5-10 membered heteroaryl, wherein each of said phenyl and heteroaryl are optionally substituted with one or more (C₁-C₄)alkyl, (CH₂)ₙC₃₋₆ cycloalkyl, (CH₂)ₙOR^{a}, (C₁-C₄)haloalkyl or halo;
each R³⁰ is independently halo, C₁₋₄ alkyl, C₁₋₄ fluoroalkyl, C₃₋₅ cycloalkyl, (CH₂)₀₋₁OR^{a}, S(O)ᵢR^{b}, or (CH₂)₀₋₁NR^{a}R^{b};
each R^{a}, R^{b} and R^{c} is independently —H, C₁₋₄ alkyl, or benzyl, wherein benzyl is optionally substituted with one or more methyl or methoxyl;
R^{d} is H or C₁₋₄ alkyl; and
each n, m, o, p and i are independently 0, 1 or 2.

2. The compound of claim 1, wherein the compound is represented by the following structural formula: 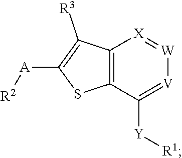
or pharmaceutically acceptable salt thereof.

3. The compound of claim 1, wherein the compound is represented by the following structural formula: 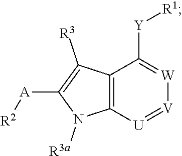
or pharmaceutically acceptable salt thereof.

4. The compound of claim 1, wherein the compound is represented by the following structural formula: 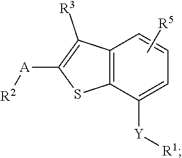
or pharmaceutically acceptable salt thereof.

5. The compound of claim 1, wherein the compound is represented by the following structural formula: 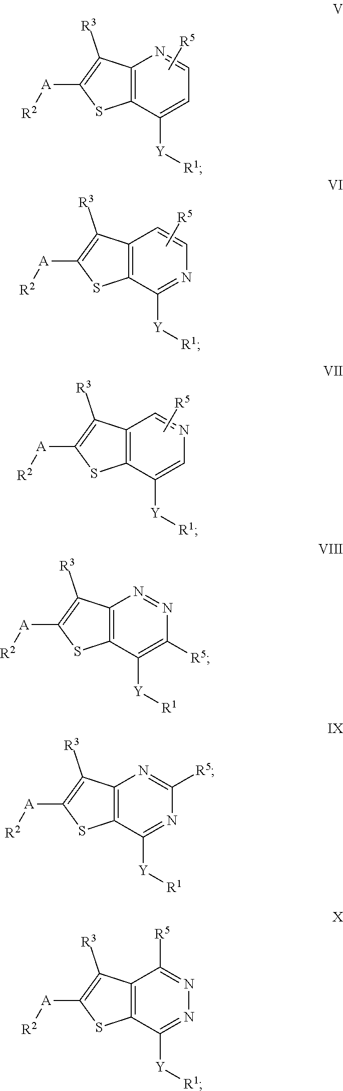
or pharmaceutically acceptable salt thereof.

6. The compound of claim 1, wherein the compound is represented by the following structural formula: 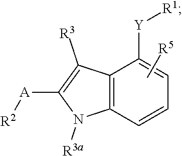
or pharmaceutically acceptable salt thereof.

7. The compound of claim 1, wherein the compound is represented by the following structural formula: 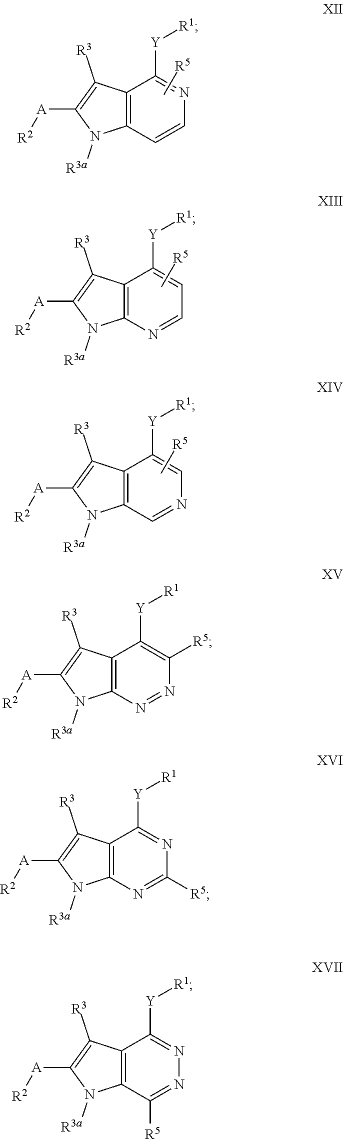
or pharmaceutically acceptable salt thereof.

8. The compound of claim 1, or pharmaceutically acceptable salt thereof, wherein:
A is represented by: 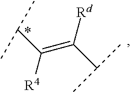
wherein “*” indicates the point of attachment to R².

9. The compound of claim 1, or pharmaceutically acceptable salt thereof, wherein:
A is represented by: 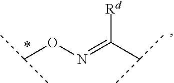
wherein “*” indicates the point of attachment to R².

10. The compound of claim 1, or a pharmaceutically acceptable salt thereof, wherein Y is O, NH or NHCH₂\hat{ }.

11. The compound of claim 1, or a pharmaceutically acceptable salt thereof, wherein Y is NH.

12. The compound of claim 1, or a pharmaceutically acceptable salt thereof, wherein R³ is (CH₂)ₒC(O)(C₁₋₆ alkyl), (CH₂)ₒS(C₁₋₆ alkyl), C₁₋₆ alkyl, (CH₂)ₒC₃₋₈ cycloalkyl, C₂₋₆ alkenyl, or C₂₋₆ alkynyl, wherein o is 0 or 1; and the C₁₋₆ alkyl represented by R³ or the C₁₋₆ alkyl, C₃₋₈ cycloalkyl, C₂₋₆ alkenyl and C₂₋₆ alkynyl in the group represented by R³ are optionally substituted with hydroxyl and/or one or more halo groups.

13. The compound of claim 1, or a pharmaceutically acceptable salt thereof, wherein R³ is C₁₋₄ alkyl or S(C₁₋₄ alkyl), wherein the C₁₋₄ alkyl represented by R³ or the C₁₋₄ alkyl in the group represented by R³ is substituted by one or more fluoro.

14. The compound of claim 1, or a pharmaceutically acceptable salt thereof, wherein R³ is CH₂CF₃.

15. The compound of claim 1, or a pharmaceutically acceptable salt thereof, wherein R³ is SCF₃.

16. The compound of claim 1, or a pharmaceutically acceptable salt thereof, wherein:
R³ is H; and
R^{3a} is (CH₂)ₒC(O)(C₁₋₆ alkyl), (CH₂)ₒS(C₁₋₆ alkyl), C₁₋₆ alkyl, (CH₂)ₒC₃₋₈ cycloalkyl, C₂₋₆ alkenyl, or C₂₋₆ alkynyl, wherein o is 0 or 1; and the C₁₋₆ alkyl represented by R^{3a} or the C₁₋₆ alkyl, C₃₋₈ cycloalkyl, C₂₋₆ alkenyl and C₂₋₆ alkynyl in the group represented by R^{3a} are optionally substituted with hydroxyl and/or one or more halo groups.

17. The compound of claim 1, or a pharmaceutically acceptable salt thereof, wherein R^{3a} is C₁₋₄ alkyl or S(C₁₋₄ alkyl), wherein the C₁₋₄ alkyl represented by R^{3a} or the C₁₋₄ alkyl in the group represented by R^{3a} is substituted by one or more fluoro.

18. The compound of claim 1, or a pharmaceutically acceptable salt thereof, wherein R^{3a} is CH₂CF₃.

19. The compound of claim 1, or a pharmaceutically acceptable salt thereof, wherein each R⁵ is independently H, C₁₋₄ alkyl, or halo.

20. The compound of claim 1, or a pharmaceutically acceptable salt thereof, wherein R⁵ is H.

21. The compound of claim 1, or a pharmaceutically acceptable salt thereof, wherein
R² is (CH₂)ₘOR¹², (CH₂)ₘN(R¹²)₂, (CH₂)ₘCN, (CH₂)ₘC(O)N(R¹²)₂, (CH₂)ₘNHC(O)R¹², (CH₂)ₘNHC(O)OR¹², (CH₂)ₘNHS(O)ᵢR¹², C₁₋₆ alkyl, (CH₂)ₘC₃₋₈ cycloalkyl, (CH₂)ₘC₆₋₁₀ aryl, 5-10 membered heteroaryl or (CH₂)ₘ(4-10 membered heterocyclyl), wherein
said alkyl is optionally substituted by one or more R^{22b};
said aryl and heteroaryl are each optionally and independently substituted by one or more R²²; and
said cycloalkyl and heterocyclyl are each optionally substituted by one or more R^{22a}, and
R² and R⁴ are optionally taken together with their intervening atom to form a C₃₋₆ cycloalkyl, or 4-6 membered heterocyclyl containing one ring nitrogen atom or one ring oxygen atom, wherein said cycloalkyl or heterocyclyl are each optionally and independently substituted by one or more R²²;
R⁴ is H, halo, or C₁₋₄ alkyl;
R¹² are each independently H, C₁₋₆ alkyl, C₃₋₈ cycloalkyl or phenyl, 5-6 membered heteroaryl; wherein said alkyl, cycloalkyl, phenyl and heteroaryl are optionally and independently substituted by one or more R³⁰,
R²² are each independently halo, CN, NO₂, (CH₂)ₙOR^{a}, S(O)ᵢR^{b}, (CH₂)ₙNR^{b}R^{c}, NHS(O)ᵢR^{c}, C(═O)OR^{b}, C(═O)NR^{b}R^{c}, NHC(═O)R^{c}, NH(C═O)OR^{c}, O(C═O)NR^{b}R^{c}, C(═O)R^{b}, halo(C₁-C₅)alkyl, or (C₁-C₅)alkyl;
R^{22a} are each independently halo, CN, NO₂, (CH₂)ₙOR^{a}, (CH₂)ₙNR^{b}R^{c}, S(O)ᵢR^{b}, NHS(O)ᵢR^{c}, S(O)ᵢNR^{b}R^{c}, C(═O)OR^{b}, C(═O)NR^{b}R^{c}, NHC(═O)R^{c}, NH(C═O)OR^{c}, O(C═O)NR^{b}R^{c}, C(═O)R^{b}, halo(C₁-C₅)alkyl, (C₁-C₅)alkyl, or two R^{22a} groups on the same ring atom taken together with their intervening atom are a (C₃₋₆)spirocycloalkyl or oxo, or two R^{22a} on adjacent ring atoms, taken together their intervening atoms form a phenyl, or a 5-6 membered heteroaryl, wherein said phenyl or heteroaryl is optionally substituted with one or more groups selected from halo, methyl, halomethyl, methoxy or halomethoxy; and
R^{22b} are each independently halo, CN, NO₂, (CH₂)ₙOR^{a}, (CH₂)ₙNR^{b}R^{c}, NHS(O)ᵢR^{c}, OC(═O)OR^{b}, NHC(═O)R^{c}, NH(C═O)OR^{c}, O(C═O)NR^{b}R^{c}, NH(C═O)NR^{b}R^{c}, halo(C₁-C₅)alkyl, (C₁-C₅)alkyl, phenyl or 5-10 membered heteroaryl, where each said phenyl and heteroaryl are optionally substituted with one or more (C₁-C₄)alkyl, (CH₂)ₙC₃₋₆ cycloalkyl, (CH₂)ₙOR^{a}, (C₁-C₄)haloalkyl or halo.

22. The compound of claim 1, wherein:
R² is (CH₂)ₘNHR¹², (CH₂)ₘC(O)N(R¹²)₂, (CH₂)ₘNHC(O)R¹², (CH₂)ₘNHC(O)OR¹², (CH₂)ₘNHS(O)ᵢR¹², C₁₋₄ alkyl, (CH₂)ₘC₃₋₆ cycloalkyl, (CH₂)ₘ phenyl, 5-10 membered heteroaryl, or (CH₂)ₘ(4-6 membered heterocyclyl), wherein said heteroaryl is selected from pyrazolyl, imidazolyl, pyridyl, pyrimidyl, oxazolyl, oxadiazolyl, isoxazolyl, thiazolyl, triazolyl, tetrazolyl, pyridin-2-onyl, pyrazolopyridyl, benzimidazolyl, benzothiazolyl, indolyl, azaindolyl, indazolyl, azaindazolyl, or benzoxazolyl, and wherein said heterocyclyl is selected from pyrrolidinonyl, pyrrolidinyl, oxazolidin-2-onyl, 1,3-dioxol-2-onyl, pyridin-2 (1H)-onyl, tetrahydrofuranyl, tetrahydropyranyl, piperidinyl, piperazinyl, morpholinyl, azetidinyl, or oxetanyl, and wherein
said alkyl is optionally substituted by one or more R^{22b};
said phenyl and heteroaryl are each optionally and independently substituted by one or more R²²;
said cycloalkyl and heterocyclyl are each optionally substituted by one or more R^{22a},
R¹² are each independently H, C₁₋₄ alkyl, C₃₋₆ cycloalkyl, phenyl, 5-6 membered heteroaryl, wherein said heteroaryl is selected from pyrazolyl, pyridyl, oxazolyl, isoxazolyl, thiazolyl, triazolyl, tetrazolyl, or imidazolyl, and wherein said alkyl, cycloalkyl, phenyl, or heteroaryl is optionally substituted with one or more groups selected from halo, C₁₋₄ alkyl, C₁₋₄ alkoxyl, S(O)ᵢR^{b}, C₁₋₄ fluoroalkyl, or C₃₋₅ cycloalkyl; and
R² and R⁴ are optionally taken together with their intervening atom to form a C₃₋₆cycloalkyl, or 4-6 membered heterocyclyl containing one ring nitrogen atom or one ring oxygen atom, wherein said cycloalkyl or heterocyclyl are each optionally and independently substituted by one or more R²²;
R⁴ is H, halo, or C₁₋₄ alkyl;
R²² are each independently halo, (CH₂)ₙOR^{a}, S(O)ᵢR^{b}, NR^{b}R^{c}, NHS(O)ᵢR^{c}, C(═O)OR^{b}, C(═O)NR^{b}R^{c}, NHC(═O)R^{c}, NH(C═O)OR^{c}, O(C═O)NR^{b}R^{c}, C(═O)R^{b}, halo(C₁-C₄)alkyl, or (C₁-C₄)alkyl;
R^{22a} are each independently halo, (CH₂)ₙOR^{a}, (CH₂)ₙNR^{b}R^{c}, S(O)ᵢR^{b}, NHS(O)ᵢR^{c}, S(O)ᵢNR^{b}R^{c}, C(═O)OR^{b}, C(═O)NR^{b}R^{c}, NHC(═O)R^{c}, NH(C═O)OR^{c}, O(C═O)NR^{b}R^{c}, C(═O)R^{b}, halo(C₁-C₄)alkyl, or (C₁-C₄)alkyl; and
R^{22b} are each independently halo, (CH₂)ₙOR^{a}, (CH₂)NR^{b}R^{c}, NHS(O)ᵢR^{c}, OC(═O)OR^{b}, NHC(═O)R^{c}, NH(C═O)OR^{c}, O(C═O)NR^{b}R^{c}, NH(C═O)NR^{b}R^{c}, halo(C₁-C₄)alkyl, (C₁-C₄)alkyl, pyrazolyl, pyridyl, pyridin-2-onyl, pyrazolopyridyl, oxadiazolyl, isoxazolyl, triazolyl, benzimidazolyl, benzothiazolyl, or phenyl, wherein said pyrazolyl, pyridyl, pyridin-2-onyl, pyrazolopyridyl, oxadiazolyl, isoxazolyl, triazolyl, benzimidazolyl, benzothiazolyl, or phenyl are each optionally and independently substituted with one or more (C₁-C₄)alkyl, CH₂ (C₃-C₆) cycloalkyl, (CH₂)ₙOR^{a}, (C₁-C₄)haloalkyl or halo.

23. The compound of claim 1, wherein:
R² is:
(CH₂)ₘNHR¹², (CH₂)ₘC(O)N(R¹²)₂, (CH₂)ₘNHC(O)R¹², (CH₂)ₘNHC(O)OR¹² or (CH₂)ₘNHS(O)ᵢR¹², wherein R¹² is H, C₁₋₄ alkyl, C₃₋₆cycloalkyl, pyrazolyl or phenyl, each optionally substituted with C₁₋₄ alkyl;
(CH₂)ₘ phenyl, wherein phenyl is optionally substituted with one or more groups selected from NR^{b}R^{c} and C(═O)NR^{b}R^{c};
C₁₋₄ alkyl optionally substituted with one or more groups selected from halo, OR^{a}, NR^{b}R^{c}, pyrazolyl, pyridyl, pyridin-2-onyl, pyrazolopyridyl, oxadiazolyl, isoxazolyl, triazolyl, benzimidazolyl, benzothiazolyl, or phenyl, wherein said pyrazolyl, pyridyl, pyridin-2-onyl, pyrazolopyridyl, oxadiazolyl, isoxazolyl, triazolyl, benzimidazolyl, benzothiazolyl, or phenyl are each optionally and independently substituted with halo, methyl, cyclopropylmethyl, or methoxyl;
(CH₂)_{0 or 1} C₃₋₆ cycloalkyl, (CH₂)_{0 or 1} 1,3-dioxol-2-onyl, (CH₂)_{0 or 1} pyrrolidinonyl, (CH₂)_{0 or 1} pyrrolidinyl, (CH₂)_{0 or 1} oxazolidin-2-onyl, (CH₂)_{0 or 1} tetrahydropyranyl, (CH₂)_{0 or 1} tetrahydrofuranyl, (CH₂)_{0 or 1} pyridin-2 (1H)-onyl or (CH₂)_{0 or 1} piperidinyl, each optionally and independently substituted by one or more groups selected from (CH₂)ₙOR^{a}, C₁₋₄ alkyl, C(O)OCH₂ (phenyl) and S(O)ᵢR^{b}; and
R² and R⁴ are optionally taken together with their intervening atom to form a (C₃-C₆) cycloalkyl, piperidinyl or tetrahyropyranyl, each optionally and independently substituted by one or more methyl, C(O)(C₁₋₄ alkyl) or NHS(O)₂ (C₁₋₄ alkyl); and
R⁴ is H or C₁₋₄ alkyl.

24. The compound of claim 1, or a pharmaceutically acceptable salt thereof, wherein R² is Me, (CH₂)ₘNHR¹², (CH₂)ₘC(O)N(R¹²)₂, (CH₂)ₘNHC(O)R¹², (CH₂)ₘNHC(O)OR¹², (CH₂)ₘNHS(O)₂R¹², or is represented by 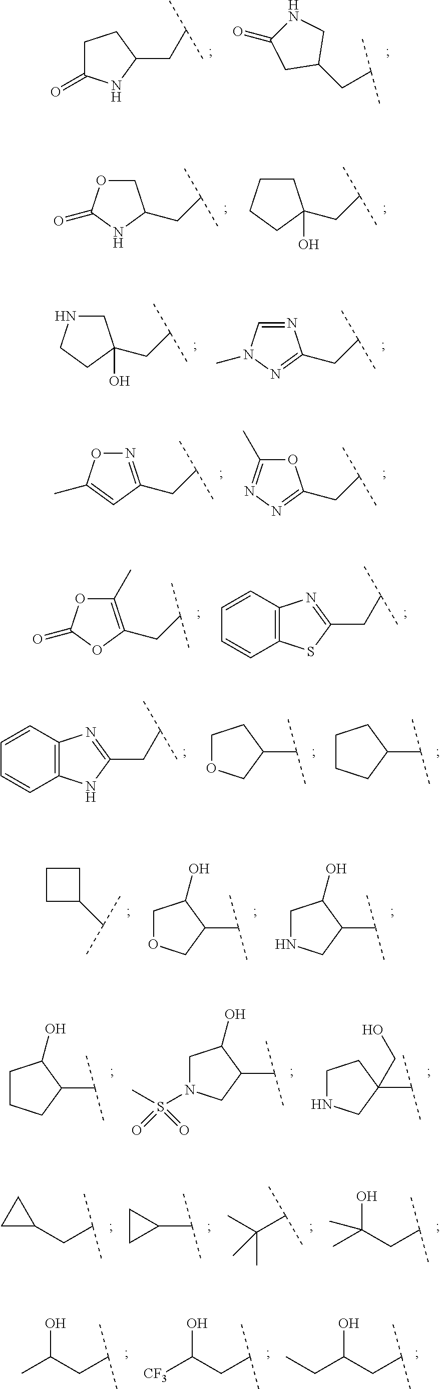 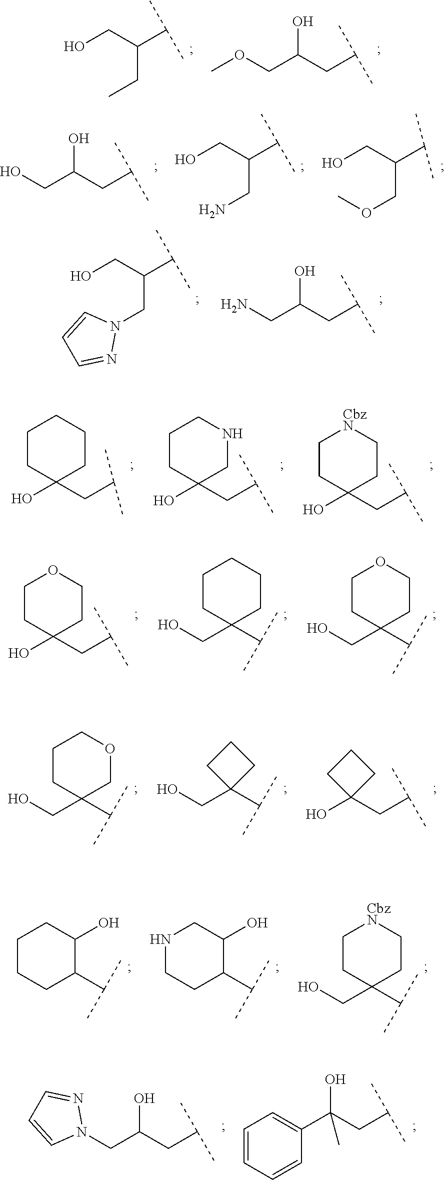 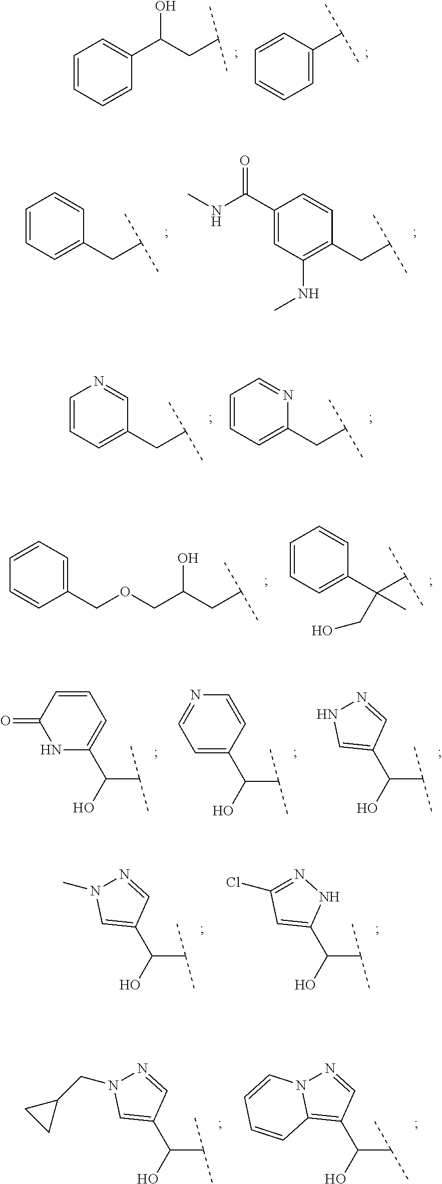 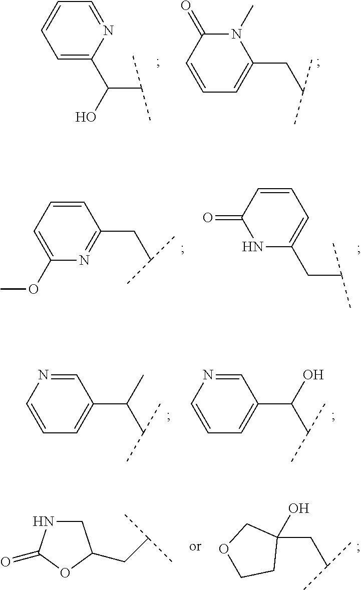
each R¹² is independently H or Me, or is represented by 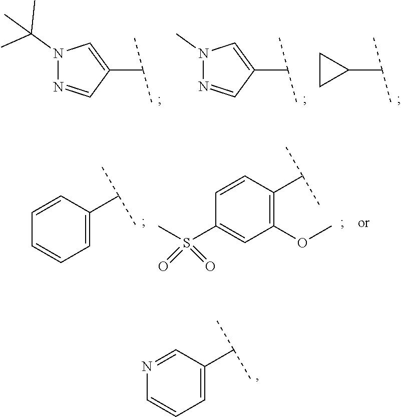
wherein R² and R⁴ are taken together with their intervening atom to form a ring represented by: 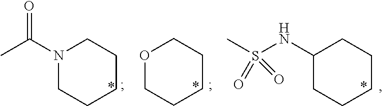
wherein “*” indicates the intervening atom between R² and R⁴; and
R⁴ is H or Me.

25. The compound of claim 1, or a pharmaceutically acceptable salt thereof, wherein R¹ is a 4 to 8 membered monocyclic heterocyclyl, a 6 to 9 membered fused bicyclic heterocyclyl or a 6 to 9 membered bridged bicyclic heterocyclyl, each heterocyclyl optionally substituted with one or more groups selected from C₁₋₄ alkyl, C₁₋₄ haloalkyl, halo, OR^{a}, NR^{a}R^{b} or a 4-6 membered heterocyclyl containing one ring oxygen atom; or is represented by

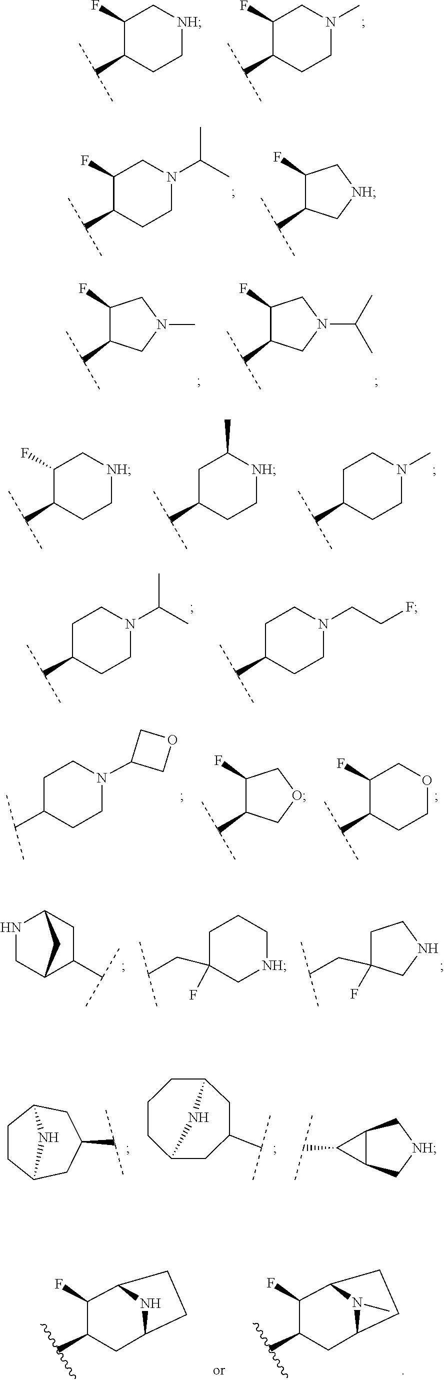

26. The compound of claim 1, or a pharmaceutically acceptable salt thereof, wherein R¹ is a 4 to 7 membered monocyclic heterocyclyl containing one nitrogen optionally substituted with C₁₋₄ alkyl or fluoro.

27. The compound of claim 1, or a pharmaceutically acceptable salt thereof, wherein R¹ is represented by:

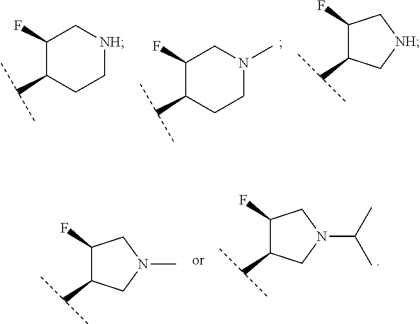

28. The compound of claim 1, or a pharmaceutically acceptable salt thereof, wherein
R^{a} is H, Me, or benzyl;
R^{b} is H or Me;
R^{c} is H or Me; and
R^{d} is H.

29. The compound of claim 1, or a pharmaceutically acceptable salt thereof, wherein
Y is NH;
when Z is NR^{3a}, R³ is H and R³ is CH₂CF₃ or SCF₃;
when Z is S, R³ is CH₂CF₃ or SCF₃;
R⁵ is H;
R² is Me, (CH₂)ₘC(O)N(R¹²)₂, or is represented by 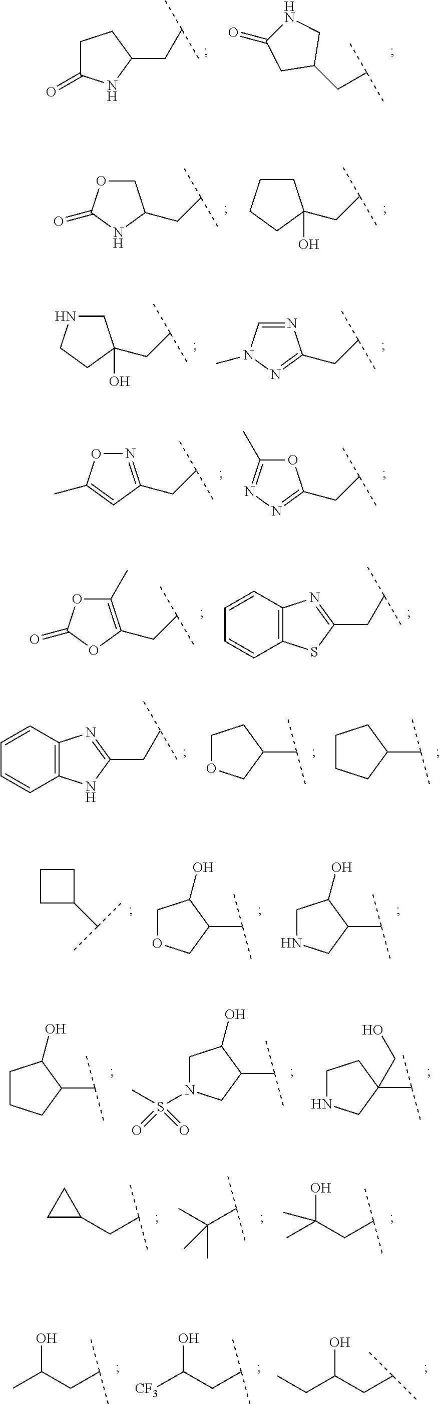 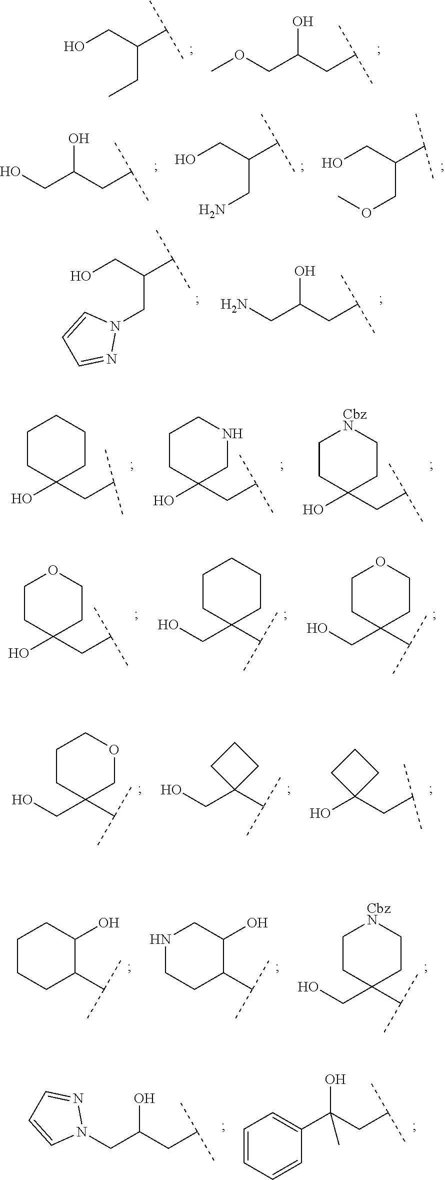 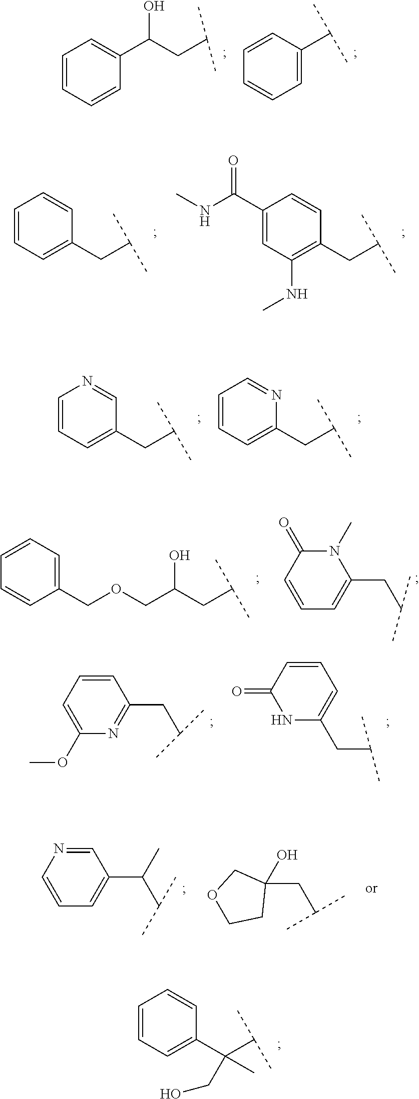
each R¹² is independently H, Me, or phenyl;
R¹ is represented by: 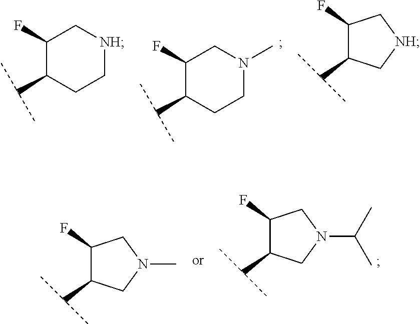
and
R^{a} is H.

30. A pharmaceutical composition comprising: i) the compound of claim 1 or a pharmaceutically acceptable salt thereof; and ii) a pharmaceutically acceptable carrier, excipient or diluent.

31. A method of treating a subject with cancer, comprising administering to the subject an effective amount of the compound of claim 1 or a pharmaceutically acceptable salt thereof.

32. The method of claim 31, wherein the cancer is characterized by dysfunctional p53.

33. The method of claim 31, wherein the cancer is characterized by an inactivating p53 mutation.

34. The method of claim 33, wherein the p53 mutation is Val143, His168, Arg175, Tyr220, Gly245, Arg248, Arg249, Phe270, Arg273, Arg282, and/or a combination thereof.

35. The method according to claim 33, wherein the p53 mutant is V157F, R175H, Y220C, G245S, R248Q, R248W, R249S, R273H, R273C, R282W, and/or a combination thereof.

36. The method of claim 33, wherein the p53 mutation is Y220C.

37. The method of claim 31, wherein the cancer is acute lymphoblastic leukemia, acute myeloid leukemia, adrenocortical carcinoma, AIDS-related cancers, AIDS-related lymphoma, anal cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, central nervous system lymphoma, cerebellar astrocytoma, cervical cancer, colon cancer, gallbladder cancer, gastric cancer, head and neck cancer, heart cancer, hepatocellular (liver) cancer, kidney cancer, liver cancer, lung cancers, such as non-small cell and small cell lung cancer, ovarian cancer, ovarian epithelial cancer, ovarian germ cell tumor, pancreatic cancer, pancreatic cancer islet cell, prostate cancer, rectal cancer, renal cell carcinoma, skin cancers, skin carcinoma merkel cell, small intestine cancer or throat cancer.

38. A method for re-activating p53 Y220C mutant in a subject in need thereof, comprising administering to the subject an effective amount of the compound of claim 1 or a pharmaceutically acceptable salt thereof.
